SEQUENCE LISTING (53 sequences)

SEQ ID NO: 1 - protein, 1468 aa, Homo sapiens
MEDHMFGVQQIQPNVISVRLFKRKVGGLGFLVKERVSKPPVIISDLIRGGAAEQSGLIQAGDIILAVNGRPLVDLSYDSALEVLRGIASETHVVLILRGPEGFTTHLETTFTGDGTPKTIRVTQPLGPPTKAVDLSHQPPAGKEQPLAVDGASGPGNGPQHAYDDGQEAGSLPHANGLAPRPPGQDPAKKATRVSLQGRGENNELLKEIEPVLSLLTSGSRGVKGGAPAKAEMKDMGIQVDRDLDGKSHKPLPLGVENDRVFNDLWGKGNVPVVLNNPYSEKEQPPTSGKQSPTKNGSPSKCPRFLKVKNWETEVVLTDTLHLKSTLETGCTEYICMGSIMHPSQHARRPEDVRTKGQLFPLAKEFIDQYYSSIKRFGSKAHMERLEEVNKEIDTTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKPPRGRFDVLPLLLQANGNDPELFQIPPELVLEVPIRHPKFEWFKDLGLKWYGLPAVSNMLLEIGGLEFSACPFSGWYMGTEIGVRDYCDNSRYNILEEVAKKMNLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKVMSMEEYDIVHLEHETLVLVVTSTFGNGDPPENGEKFGCALMEMRHPNSVQEERKYPEPLRFFPRKGPPLPNGDTEVHGLAAARDSQHRSYKVRFNSVSSYSDSQKSSGDGPDLRDNFESAGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTFVAEAPELTQGLSNVHKKRVSAARLLSRQNLQSPKSSRSTIFVRLHTNGSQELQYQPGDHLGVFPGNHEDLVNALIERLEDAPPVNQMVKVELLEERNTALGVISNWTDELRLPPCTIFQAFKYYLDITTPPTPLQLQQFASLATSEKEKQRLLVLSKGLQEYEEWKWGKNPTIVEVLEEFPSIQMPATLLLTQLSLLQPRYYSISSSPDMYPDEVHLTVAIVSYRTRDGEGPIHHGVCSSWLNRIQADELVPCFVRGAPSFHLPRNPQVPCILVGPGTGIAPFRSFWQQRQFDIQHKGMNPCPMVLVFGCRQSKIDHIYREETLQAKNKGVFRELYTAYSREPDKPKKYVQDILQEQLAESVYRALKEQGGHIYVCGDVTMAADVLKAIQRIMTQQGKLSAEDAGVFISRMRDDNRYHEDIFGVTLRTYEVTNRLRSESIAFIEESKKDTDEVFSS

SEQ ID NO: 2 - DNA, 12291 bp, Homo sapiens
ataaaagatgtatgctttggagcccagagcggctcttttaatgagggttgcgacgtctccctccccacacccataaaccagtcgggttggacgtcactgctaattcgtttcagtgatgataggataaaggagggacattaagaaataaattccccctcacgaccctcgctgagctcacggctcagtccctacatatttatgccgcgtttccagccgctgggtgaggagctacttagcgccgcggctcctccgaggggcggccgggcagcgagcagcggccgagcggacgggctcatgatgcctcagatctgatccgcatctaacaggctggcaatgaagatacccagagaatagttcacatctatcatgcgtcacttctagacacagccatcagacgcatctcctcccctttctgcctgaccttagggacacgtcccaccgcctctcttgacgtctgcctggtcaaccatcacttccttagagaataaggagagaggcggatgcaggaaatcatgccaccgacgggccaccagccatgagtgggtgacgctgagctgacgtcaaagacagagagggctgaagccttgtcagcacctgtcaccccggctcctgctctccgtgtagcctgaagcctggatcctcctggtgaaatcatcttggcctgatagcattgtgaggtcttcagacaggacccctcggaagctagttaccatggaggatcacatgttcggtgttcagcaaatccagcccaatgtcatttctgttcgtctcttcaagcgcaaagttgggggcctgggatttctggtgaaggagcgggtcagtaagccgcccgtgatcatctctgacctgattcgtgggggcgccgcagagcagagtggcctcatccaggccggagacatcattcttgcggtcaacggccggcccttggtggacctgagctatgacagcgccctggaggtactcagaggcattgcctctgagacccacgtggtcctcattctgaggggccctgaaggtttcaccacgcacctggagaccacctttacaggtgatgggacccccaagaccatccgggtgacacagcccctgggtccccccaccaaagccgtggatctgtcccaccagccaccggccggcaaagaacagcccctggcagtggatggggcctcgggtcccgggaatgggcctcagcatgcctacgatgatgggcaggaggctggctcactcccccatgccaacggcctggcccccaggcccccaggccaggaccccgcgaagaaagcaaccagagtcagcctccaaggcagaggggagaacaatgaactgctcaaggagatagagcctgtgctgagccttctcaccagtgggagcagaggggtcaagggaggggcacctgccaaggcagagatgaaagatatgggaatccaggtggacagagatttggacggcaagtcacacaaacctctgcccctcggcgtggagaacgaccgagtcttcaatgacctatgggggaagggcaatgtgcctgtcgtcctcaacaacccatattcagagaaggagcagccccccacctcaggaaaacagtcccccacaaagaatggcagcccctccaagtgtccacgcttcctcaaggtcaagaactgggagactgaggtggttctcactgacaccctccaccttaagagcacattggaaacgggatgcactgagtacatctgcatgggctccatcatgcatccttctcagcatgcaaggaggcctgaagacgtccgcacaaaaggacagctcttccctctcgccaaagagtttattgatcaatactattcatcaattaaaagatttggctccaaagcccacatggaaaggctggaagaggtgaacaaagagatcgacaccactagcacttaccagctcaaggacacagagctcatctatggggccaagcacgcctggcggaatgcctcgcgctgtgtgggcaggatccagtggtccaagctgcaggtattcgatgcccgtgactgcaccacggcccacgggatgttcaactacatctgtaaccatgtcaagtatgccaccaacaaagggaacctcaggtctgccatcaccatattcccccagaggacagacggcaagcacgacttccgagtctggaactcccagctcatccgctacgctggctacaagcagcctgacggctccaccctgggggacccagccaatgtgcagttcacagagatatgcatacagcagggctggaaaccgcctagaggccgcttcgatgtcctgccgctcctgcttcaggccaacggcaatgaccctgagctcttccagattcctccagagctggtgttggaagttcccatcaggcaccccaagtttgagtggttcaaggacctggggctgaagtggtacggcctccccgccgtgtccaacatgctcctagagattggcggcctggagttcagcgcctgtcccttcagtggctggtacatgggcacagagattggtgtccgcgactactgtgacaactcccgctacaatatcctggaggaagtggccaagaagatgaacttagacatgaggaagacgtcctccctgtggaaggaccaggcgctggtggagatcaatatcgcggttctctatagcttccagagtgacaaagtgaccattgttgaccatcactccgccaccgagtccttcattaagcacatggagaatgagtaccgctgccgggggggctgccctgccgactgggtgtggatcgtgccccccatgtccggaagcatcacccctgtgttccaccaggagatgctcaactaccggctcaccccctccttcgaataccagcctgatccctggaacacgcatgtctggaaaggcaccaacgggacccccacaaagcggcgagccattggcttcaagaagctagcagaagctgtcaagttctcggccaagctgatggggcaggctatggccaagagggtgaaagcgaccatcctctatgccacagagacaggcaaatcgcaagcttatgccaagaccttgtgtgagatcttcaaacacgcctttgatgccaaggtgatgtccatggaagaatatgacattgtgcacctggaacatgaaactctggtccttgtggtcaccagcacctttggcaatggagatccccctgagaatggggagaaattcggctgtgctttgatggaaatgaggcaccccaactctgtgcaggaagaaaggaagtacccggaacccttgcgtttctttccccgtaaagggcctcccctccccaatggtgacacagaagtccacggtctggctgcagcccgtgacagccagcacaggagctacaaggtccgattcaacagcgtctcctcctactctgactcccaaaaatcatcaggcgatgggcccgacctcagagacaactttgagagtgctggacccctggccaatgtgaggttctcagtttttggcctcggctcacgagcataccctcacttttgcgccttcggacacgctgtggacaccctcctggaagaactgggaggggagaggatcctgaagatgagggaaggggatgagctctgtgggcaggaagaggctttcaggacctgggccaagaaggtcttcaaggcagcctgtgatgtcttctgtgtgggagatgatgtcaacattgaaaaggccaacaattccctcatcagcaatgatcgcagctggaagagaaacaagttccgcctcacctttgtggccgaagctccagaactcacacaaggtctatccaatgtccacaaaaagcgagtctcagctgcccggctccttagccgtcaaaacctccagagccctaaatccagtcggtcaactatcttcgtgcgtctccacaccaacgggagccaggagctgcagtaccagcctggggaccacctgggtgtcttccctggcaaccacgaggacctcgtgaatgccctgatcgagcggctggaggacgcgccgcctgtcaaccagatggtgaaagtggaactgctggaggagcggaacacggctttaggtgtcatcagtaactggacagacgagctccgcctcccgccctgcaccatcttccaggccttcaagtactacctggacatcaccacgccaccaacgcctctgcagctgcagcagtttgcctccctagctaccagcgagaaggagaagcagcgtctgctggtcctcagcaagggtttgcaggagtacgaggaatggaaatggggcaagaaccccaccatcgtggaggtgctggaggagttcccatctatccagatgccggccaccctgctcctgacccagctgtccctgctgcagccccgctactattccatcagctcctccccagacatgtaccctgatgaagtgcacctcactgtggccatcgtttcctaccgcactcgagatggagaaggaccaattcaccacggcgtatgctcctcctggctcaaccggatacaggctgacgaactggtcccctgtttcgtgagaggagcacccagcttccacctgccccggaacccccaagtcccctgcatcctcgttggaccaggcaccggcattgcccctttccgaagcttctggcaacagcggcaatttgatatccaacacaaaggaatgaacccctgccccatggtcctggtcttcgggtgccggcaatccaagatagatcatatctacagggaagagaccctgcaggccaagaacaagggggtcttcagagagctgtacacggcttactcccgggagccagacaaaccaaagaagtacgtgcaggacatcctgcaggagcagctggcggagtctgtgtaccgagccctgaaggagcaagggggccacatatacgtctgtggggacgtcaccatggctgctgatgtcctcaaagccatccagcgcatcatgacccagcaggggaagctctcggcagaggacgccggcgtattcatcagccggatgagggatgacaaccgataccatgaggatatttttggagtcaccctgcgaacgtacgaagtgaccaaccgccttagatctgagtccattgccttcattgaagagagcaaaaaagacaccgatgaggttttcagctcctaactggaccctcttgcccagccggctgcaagttttgtaagcgcggacagacactgctgaacctttcctctgggaccccctgtggccctcgctctgcctcctgtccttgtcgctgtgccctggtttccctcctcgggcttctcgcccctcagtggtttcctcggccctcctgggtttactccttgagttttcctgctgcgatgcaatgcttttctaatctgcagtggctcttacaaaactctgttcccactccctctcttgccgacaagggcaactcacgggtgcatgaaaccactggaacatggccgtcgctgtgggggtttttttctctggggttcccctggaaaggctgcaggaactaggcacaagctctctgagccagtccctcagccactgaagtccccctttctccttttttatgatgacattttggttgtgcgtgcctgtgtgtgtgtgtgtgtgtgtgtgtgtgtgtgtgatgggccaggtctctgtccgtcctcttccctgcacaagtgtgtcgatcttagattgccactgctttcattgaagaccctcaatgccaagaaacgtgtccctggcccatattaatccctcgtgtgtccataattagggtccacgcccatgtacctgaaacatttggaagccccataattgttctagttagaaagggttcagggcatggggagaggagtgggaaattgattaaaggggctgtctcccaatgaaagaggcattcccagaatttgctgcatttagattttgataccagtgagcagagccctcatgtgacatgaacccatccaatggattgtgcaaatcccctccccaaacccacccataccagctagaatcacttgactttgccacatccattgactgaccccctcctccagcaatagcatccaaggggcctggaagttatgttgttcaaagaagcctggtggcaataaggatcttcccactttgccactggatgactttggatgggtcacttgtcctcagtttttcctagtcataatgtcatacgaacctaaagaatatgaatggattaaatgttaaagctttggtgcctggaaacaatatcaagtaacaatatgattattatttttttattcccccaaagcgggcttgctgcttcacccttggggatgaaataatggaagctggttaaagtggatgaggttggaaagagttgccataatgaggtcccacgtggcttcttcgataggagccacaacttggggtgggaagaacttgtccctcaggcttgttgccctctgcagttgatctccaaagttttaaacctgttaaattaattttgacaaataagttaccctcaactcagatcaaaaatgggcagccaagtcttcggtaggaattggagccggtgtaattcctccctaagaggcaacctgttgaatttactctctcagagtaaatggtgggaagggatccctttgtatacttttttaaatactacaaattagtgtcaggcagttcccagaaagagacaagaaatcctagtggcctcccagactgcagggtccccaaggatggaaagggaatgttctgctggttctaccctgtttgttgtgtcttgctatacagaaaaaccacatttcttttatatactgtacgtgggcatatcttgttgttcagtttgggtgtctgctaaagaggaagtgcactggccctctttgaaagggctttacagtgggggcaccaagaccccaaagggcccaggccaggagactgttaaagtgaaaaggcaatctatgactcaccttgctctgccatccctggcagcccccaccggtgtcctgttcctgccacatggagcttgacttcatgccagctataatctcccctgccttcctttaatcccaatttcccctgctcactcttccacagatataaagaacaaacacttagcatcccacactcaccccttctaatcctgaagggaagcccattctaaactcctttcctgcaaacccatttccagctcctagtagctttcctcccaaaggctttctttccaatcctttatagctttggagacgcctccccaattccccagggaaggaaactgttgtgtccaatccccattaaagacaaattgatcagtgcttcccactccaagtcaagctttatgcaggaatgcttttccatcagggaataaatacttagaagcgcttacaaggtgccaggcacctcctttctgcatgtgcctgcctttctagtagcagacagatggaaacattgtctcattttgtcaaggagtccaaagaaatgattataaaaccaggattcatccttcttctccagaaagatttttttttaagtaaacacctttcaatccccaacacaagctgcttcacaactccaggctagaaggcaggagagcgatctgatgtgtttctttcatttgccagaattcctgataccaaaagcctctctctctgttgagtaacctctcaaggaccagagtggagtccagattgttaggctcagatcaagggtggggaaatactgccctctcgtggtggcttttcatccaggcctcgtagccaaccgtttaagtgcaaaatagaattaagcaatgggtaagcaaaatagggttgacaagatatttgggggttattcgggttatggcccatttatttccctcttccccctgaattgaccagtagcagctccagccccatttcacaaaagtgagtttggccaggaggaatgagacgtctcctgaaataggaacaccggaacatcatgctcacctgccatcactatgcatccagttcccacagcttgtgtcgtgaaagagcagagagatgatgttaaactccttgggaggagagagggcttcttttggtttccctggagtgagacagccaggtgtctttcttttgcggggggacacttcagacccatcaatatggaattttgggagccgacctgagtgcaaatcctaattctgcccctgttggtgcagatggctgtgggcggctcacttgaccttttagagtctgcatacccacctgtataacaaggtggattgaatgagacaatgcccacgaaatgcccagttacagtacctggttcaaaacttactgcattttaatttttcacttaacttataacatgtcttgcttctccagtgtgtggaaggcaccgggcagtttgcagagataagcaaaacacagttcctctcgtgcagaaggttagaatctatttttttttttgacagagtcttgctctgtcacccaggctggcgtacagtggtacgatctcagctcactgcatcctctgcctcccccagttcaagtgattcttctgcctcggcctcctgagtaactgggactacaggcgcctaccaccacgcccagctaagttttgtatttttagtagagtcagggtttcaccatgttggccaggctggtcttgaattcctgacctcaaatgatccacgcacctcagcctcccaaagtgctggattacaggcatgagccaccacgcccagccaaaggttataatctgatggagagagacacccgtcttggaactgacataaatttctggggtttgagaaatgggcgggatttcactggtagcttctggaaggtaagagttgtccaggaattgggaagagtgagaggaaaggcacggacagggagcatgtaagataaattgaggctggctttggaaggctgaggagggtgagaaaaggtgggctgggaccagaccgtggggagaggtgagtggcattacaagaaatttaggctttattcagaaggcaacagggagtccctaagaatgtttttcaaaaagggacattaaggcgattggagttatacttggaaaagaaagttctggccacagtacagagcatggcccgttgagctgttgggggggttattgctgcaaccaaggcttgagtgagggaagaggcggatgtagtgataaagagactccaggaactgaatcagcgtacctggcaccccatccattgtagagggtgagaataaaggagaaattaaagcatcttgcaggctgggcgcggtagctcatgtctgtaatcccagcactttgggaggccgaggtgggtgtatcagttgaggtcaggagttggagaccagtcagccagttagtagaaaccctgactctactaagaaaatacaaaaattagctgggcatggtggcatgcgcctgtagtctcagctacctgggaggctgaggaaggaggatcgcttgagcccaggaggtggaggctgcagtgagccaagattgtaccactgcactccagcctgggtgacagagcaagactcttatctcaaaaaaaataaaataaaataaaataaaataaaacatcttgcccctagctgagagagaggtctctgaagagcaggctcagggaaaagatgagttttcagagctgatgtgatagtcagcttctctggagtcaacagggtgaatccttcccaagtccagccatgcccagatgcccggagggaaaactgacccccagccagtagacattggctaagaacacagaatcttctgaccaaacacgctttcagcagctgcctgctctggactttgaaagaggtcaggtcttgccctaagctcaaaacaagtgagaggtgtcctgacctagctcatagggcaaatggtcctaataggatgggcaatccagatgcctgagccccttcactccgacagcaccagcgcctaatgcagccttttcattcttgccattaggaaatctgtggacttctagcctgtgttttaaaccagccatgtttccttgtatatttccctacccgctgccccacatacccagcatgccgctgtggccaccatgtcctcaaagccttctgtctgtatcaggaatgtagtctgagactgccaggaagcaacaaggagagagaaacactaactagtcttcctttataacccattcatactctctggctgtccccaaccttcatagtctcctgcatccaaatgtcctctttggctcaaaaagtaggccaggcatggtggttcatgcctgtaatagcactttgggagactgaggtgggaggatcacttggggccaggagtttgagaccagcttgggcaacacagcgcaatctcgtctctactaaaaaaaaaaaaaaaaaaaaattagctgggcatgatggcatgctcctgtggtcccagctacttgggaggctgaggcaggaggatcacttggtcccaggagtttgaggcgacagtgagctaggatcgcaccactgcactccagcctgagtgacagagcaagaccctgtctctaaaaaaaattaaaatgaaagaccaggtgctgggattaaggaaacacaggtctgagggtctgagggaaggggcctgcctcccagggagtcaacatagatgttccccatgaacagggatttgactttggaggccaacctggcctggcctctgccctttatctcacactccctatccttggcccactgccagtccctgccttgtggcaaaggggccccaaaagaaaagctgcccttccccaaatgtaaggacccaggtacactttcacccgtggaaagcagtgtctgtcgagagtctgtttcctattaatacttatcaaagccatgtgcgagggaggtggtcagctgtcaatatgccttagtatgtttatatgagtttgttttgttctaaaatacccaaacagttctggtcaagcggggctatgcccgtctggcccaaaacacagtccgttattaacgagatggccctggcaggcgggaacaaatctgcctccatgcactgcttcctgtagtcttttagaaagtaactccaggacatcgaagtgcccagatttgactcctaagttctaggagactgtagcgcagggtctgtcaaccttagcactattggcatttggggctgggtaattctttcttgtgggggccgtcttgggtactgtaggaagctgagcagcattcctggcctccatccacaagatacctgtagcagtgtcctgccaacggtaacaatcaagtatgtcatcagacattgcccaatgtccccagggggcaacacccctctcttggacttcagggtcaagagaatctctgctggctaccccaggacttctcattatagatttcctggagcacgcagcagaaactttgcctagcccagtggttgtttccattatctgctgccaaagtgggatttgagggtgtccgggggagggggcatggggagggcagtatgctttcaaaaacccctcccaggccaggcgtggtggctcatgcctgtaatcacaggactttgggaggccgaggctggcagatcacttgaggctgggagttagagaccaacctggctaacatggcaaaacctcgtctctactaaaaatacaaaaatcagcccggcgtggtggcgggcatctgtaatcccatctactcgggaggctgaggcaggagaattacttgaacccaggaggcagaggctgcagtgagccgagatggcaccactgcactccagcttgttgacagaatgagaccctgtggaaaaaaaaaaaaaagccctcccatgccagaacagaggatggcagtctgtttcaataagacactgtgtccttggtgttggttctgattaagactcactgagatccagtgctcttgagctgggtctcagtcccctcccatgtcctgtgctctgccgccactgttttcattgttgtgttctcgttgtgattgttaagactcacactcctggctcagcagtggttttccagaaggcccaaagagcggtgccgggcaccccacgtcgcagtgtccgttccgggcttgggaagctggggaggtgggcagacctggtcgcatctcaccacacacacacacacacacacacacacacacgctgtcagaaactcggccgtcccccctacctctgagctctcaatgctgctaatctctgccaagtgtccctgtgctccagcaccttccttgaaggactgacgcccaccccacgctctttgcgaggttgtccaggctgtgtttgtcgcatgctcttcttctgtatagttctcatcttccaattttatgggattcaacaaaagcctattatgcttgtttgcattatggttacaatattaaaaagtggattcaaaaaaaa

SEQ ID NO: 3 - protein, 305 aa, Homo sapiens
MEDHMFGVQQIQPNVISVRLFKRKVGGLGFLVKERVSKPPVIISDLIRGGAAEQSGLIQAGDIILAVNGRPLVDLSYDSALEVLRGIASETHVVLILRGPEGFTTHLETTFTGDGTPKTIRVTQPLGPPTKAVDLSHQPPAGKEQPLAVDGASGPGNGPQHAYDDGQEAGSLPHANGLAPRPPGQDPAKKATRVSLQGRGENNELLKEIEPVLSLLTSGSRGVKGGAPAKAEMKDMGIQVDRDLDGKSHKPLPLGVENDRVFNDLWGKGNVPVVLNNPYSEKEQPPTSGKQSPTKNGSPSKCPRF

SEQ ID NO: 4 - protein, 1163 aa, Homo sapiens
LKVKNWETEVVLTDTLHLKSTLETGCTEYICMGSIMHPSQHARRPEDVRTKGQLFPLAKEFIDQYYSSIKRFGSKAHMERLEEVNKEIDTTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKPPRGRFDVLPLLLQANGNDPELFQIPPELVLEVPIRHPKFEWFKDLGLKWYGLPAVSNMLLEIGGLEFSACPFSGWYMGTEIGVRDYCDNSRYNILEEVAKKMNLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKVMSMEEYDIVHLEHETLVLVVTSTFGNGDPPENGEKFGCALMEMRHPNSVQEERKYPEPLRFFPRKGPPLPNGDTEVHGLAAARDSQHRSYKVRFNSVSSYSDSQKSSGDGPDLRDNFESAGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTFVAEAPELTQGLSNVHKKRVSAARLLSRQNLQSPKSSRSTIFVRLHTNGSQELQYQPGDHLGVFPGNHEDLVNALIERLEDAPPVNQMVKVELLEERNTALGVISNWTDELRLPPCTIFQAFKYYLDITTPPTPLQLQQFASLATSEKEKQRLLVLSKGLQEYEEWKWGKNPTIVEVLEEFPSIQMPATLLLTQLSLLQPRYYSISSSPDMYPDEVHLTVAIVSYRTRDGEGPIHHGVCSSWLNRIQADELVPCFVRGAPSFHLPRNPQVPCILVGPGTGIAPFRSFWQQRQFDIQHKGMNPCPMVLVFGCRQSKIDHIYREETLQAKNKGVFRELYTAYSREPDKPKKYVQDILQEQLAESVYRALKEQGGHIYVCGDVTMAADVLKAIQRIMTQQGKLSAEDAGVFISRMRDDNRYHEDIFGVTLRTYEVTNRLRSESIAFIEESKKDTDEVFSS

SEQ ID NO: 5 - protein, 34 aa, Homo sapiens
YPEPLRFFPRKGPPLPNGDTEVHGLAAARDSQHR

SEQ ID NO: 6 - protein, 17 aa, Artificial Sequence
  Description of Artificial Sequence Synthetic peptide
KDGKKKKKKSKTKCVIM

SEQ ID NO: 7 - protein, 4 aa, Artificial Sequence
  Description of Artificial Sequence Synthetic peptide
CXXX

SEQ ID NO: 8 - protein, 15 aa, Unknown
  Description of Unknown Penetratin sequence
RQIKWFQNRRMKWKK

SEQ ID NO: 9 - protein, 11 aa, Human immunodeficiency virus
YGRKKRRQRRR

SEQ ID NO: 10 - protein, 11 aa, Human immunodeficiency virus
YARAAARQARA

SEQ ID NO: 11 - protein, 13 aa, Artificial Sequence
  Description of Artificial Sequence Synthetic peptide
GRRRRRRRRRPPQ

SEQ ID NO: 12 - protein, 10 aa, Artificial Sequence
  Description of Artificial Sequence Synthetic peptide
RRRRRRRRRR

SEQ ID NO: 13 - protein, 18 aa, Unknown
  Description of Unknown SynB1 sequence
RGGRLSYSRRRFSTSTGR

SEQ ID NO: 14 - protein, 10 aa, Unknown
  Description of Unknown SynB3 sequence
RRLSYSRRRF

SEQ ID NO: 15 - protein, 12 aa, Unknown
  Description of Unknown PTD-4 sequence
PIRRRKKLRRLK

SEQ ID NO: 16 - protein, 12 aa, Unknown
  Description of Unknown PTD-5 sequence
RRQRRTSKLMKR

SEQ ID NO: 17 - protein, 15 aa, Flock house virus
RRRRNRTRRNRRRVR

SEQ ID NO: 18 - protein, 19 aa, Brome mosaic virus
KMTRAQRRAAARRNRWTAR

SEQ ID NO: 19 - protein, 13 aa, Human T-lymphotropic virus 2
TRRQRTRRARRNR

SEQ ID NO: 20 - protein, 13 aa, Human immunodeficiency virus
GRKKRRQRRRPPQ

SEQ ID NO: 21 - protein, 27 aa, Artificial Sequence
  Description of Artificial Sequence Synthetic peptide
GWTLNSAGYLLGKINLKALAALAKKIL

SEQ ID NO: 22 - protein, 17 aa, Unknown
  Description of Unknown MAP sequence
KLALKLALKLALALKLA

SEQ ID NO: 23 - protein, 27 aa, Unknown
  Description of Unknown SBP sequence
MGLGLHLLVLAAALQGAWSQPKKKRKV

SEQ ID NO: 24 - protein, 27 aa, Unknown
  Description of Unknown FBP sequence
GALFLGWLGAAGSTMGAWSQPKKKRKV

SEQ ID NO: 25 - protein, 27 aa, Unknown
  Description of Unknown MPG sequence
GALFLGFLGAAGSTMGAWSQPKKKRKV

SEQ ID NO: 26 - protein, 27 aa, Unknown
  Description of Unknown MPG-deltaNLS sequence
GALFLGFLGAAGSTMGAWSQPKSKRKV

SEQ ID NO: 27 - protein, 21 aa, Unknown
  Description of Unknown Pep-1 sequence
KETWWETWWTEWSQPKKKRKV

SEQ ID NO: 28 - protein, 21 aa, Unknown
  Description of Unknown Pep-2 sequence
KETWFETWFTEWSQPKKKRKV

SEQ ID NO: 29 - RNA, 13 nt, Artificial Sequence
  Description of Artificial Sequence Synthetic oligonucleotide
gccgccrccaugg

SEQ ID NO: 30 - DNA, 23 bp, Artificial Sequence
  Description of Artificial Sequence Synthetic primer
ccggagtagctcaggttcctgtg

SEQ ID NO: 31 - DNA, 21 bp, Artificial Sequence
  Description of Artificial Sequence Synthetic primer
gtgggcactcagggcagccac

SEQ ID NO: 32 - DNA, 30 bp, Artificial Sequence
  Description of Artificial Sequence Synthetic primer
ccttaattaacagccaccccatatcccatg

SEQ ID NO: 33 - DNA, 30 bp, Artificial Sequence; identical to SEQ ID NO: 32
  Description of Artificial Sequence Synthetic primer

SEQ ID NO: 34 - DNA, 18 bp, Artificial Sequence
  Description of Artificial Sequence Synthetic primer
gtcttccaccaggagatg

SEQ ID NO: 35 - DNA, 21 bp, Artificial Sequence
  Description of Artificial Sequence Synthetic primer
aaaggcacagaagtgggggta

SEQ ID NO: 36 - DNA, 68 bp, Artificial Sequence
  Description of Artificial Sequence Synthetic primer
cacagatgaggttttcagctcctacccatatgacgttcctgactacgcatccctttaattaattaagg

SEQ ID NO: 37 - DNA, 32 bp, Artificial Sequence
  Description of Artificial Sequence Synthetic primer
gccttaattaattaaagggatgcgtagtcagg

SEQ ID NO: 38 - DNA, 31 bp, Artificial Sequence
  Description of Artificial Sequence Synthetic primer
gaggttttcagctcctacccatatgacgttc

SEQ ID NO: 39 - DNA, 27 bp, Artificial Sequence
  Description of Artificial Sequence Synthetic primer
cggatcgtattaattaaggcgccgcgg

SEQ ID NO: 40 - DNA, 21 bp, Artificial Sequence
  Description of Artificial Sequence Synthetic primer
gaccagccattagcagtagac

SEQ ID NO: 41 - DNA, 19 bp, Artificial Sequence
  Description of Artificial Sequence Synthetic primer
ttctcgaccaatactactc

SEQ ID NO: 42 - DNA, 19 bp, Artificial Sequence
  Description of Artificial Sequence Synthetic primer
gtgtccaacatgctgctgg

SEQ ID NO: 43 - DNA, 19 bp, Artificial Sequence
  Description of Artificial Sequence Synthetic primer
ctgtgcgagatcttcaagc

SEQ ID NO: 44 - DNA, 18 bp, Artificial Sequence
  Description of Artificial Sequence Synthetic primer
gaagctccagagctgacc

SEQ ID NO: 45 - DNA, 18 bp, Artificial Sequence
  Description of Artificial Sequence Synthetic primer
ctcacacagctgtcgctg

SEQ ID NO: 46 - DNA, 21 bp, Artificial Sequence
  Description of Artificial Sequence Synthetic primer
aatggccacaccattagcctg

SEQ ID NO: 47 - DNA, 20 bp, Artificial Sequence
  Description of Artificial Sequence Synthetic primer
tgtccttgagctggtaggtg

SEQ ID NO: 48 - DNA, 20 bp, Artificial Sequence
  Description of Artificial Sequence Synthetic primer
agtaatcacgaacgccaatc

SEQ ID NO: 49 - DNA, 20 bp, Artificial Sequence
  Description of Artificial Sequence Synthetic primer
ttgccaaaggtgctggtgac

SEQ ID NO: 50 - DNA, 19 bp, Artificial Sequence
  Description of Artificial Sequence Synthetic primer
gaagatggtcgatcggctg

SEQ ID NO: 51 - DNA, 20 bp, Artificial Sequence
  Description of Artificial Sequence Synthetic primer
gtatggtaggacacgatggc

SEQ ID NO: 52 - protein, 34 aa, Mus sp.
YPEPLRFFPRKGPSLSHADSEAHSLVAARDSQHR

SEQ ID NO: 53 - DNA, 500 bp, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2016/0220647 A1
(43) Pub. Date: Aug. 4, 2016

(54) PROTEINS FOR TARGETING NEURONAL NITRIC OXIDE SYNTHASE TO MUSCLE SARCOLEMMA AND RELATED METHODS OF USE

(71) Applicant: UNIVERSITY OF WASHINGTON THROUGH ITS CENTER FOR COMMERCIALIZATION, Seattle, WA (US)

(72) Inventors: Stanley C. FROEHNER, Seattle, WA (US); Marvin ADAMS, Kent, WA (US); Min Jeong KIM, Seattle, WA (US); Daniela Rebolledo LOPEZ, Santiago (CL); Nicholas P. WHITEHEAD, Seattle, WA (US)

(73) Assignee: UNIVERSITY OF WASHINGTON THROUGH ITS CENTER FOR COMMERCIALIZATION, Seattle, WA (US)

(21) Appl. No.: 14/916,429

(22) PCT Filed: Sep. 9, 2014
(86) PCT No.: PCT/US14/54727

§ 371 Date: Mar. 3, 2016

Related U.S. Application Data
(60) Provisional application No. 61/875,477, filed on Sep. 9, 2013.

(51) Int. Cl. A61K 38/44; C12N 9/14; A61K 38/46; C12N 9/02

(52) U.S. Cl. CPC A61K 38/44; C12N 9/0075; C12N 9/14; A61K 38/46; C07K 2319/00; A61K 48/00

(57) ABSTRACT

It is demonstrated herein that nNOS-μ is an important modulator of muscle function, and that the loss of nNOS-μ contributes to pathogenesis in neuromuscular diseases, including DMD. Furthermore, the impact of the restoration of cytoplasmic and sarcolemma-localized nNOS-μ on mdx skeletal muscle pathology and function is demonstrated. Accordingly, provided herein are compositions, including therapeutic and pharmaceutical compositions, comprising nNOS-μ fusion proteins and derivatives thereof, and methods of treating neuromuscular disorders using such nNOS-μ fusion proteins.

### CROSS REFERENCE TO RELATED APPLICATIONS

This application claims benefit under 35 U.S.C. §119(e) of U.S. Provisional No. 61/875,477, filed Sep. 9, 2013, the contents of which are incorporated herein by reference in their entirety.

### STATEMENT REGARDING FEDERALLY SPONSORED RESEARCH

This invention was made with U.S. government support under 5R01AR056221, awarded by the National Institutes of Health. The U.S. Government has certain rights in the invention.

### FIELD OF THE INVENTION

The present disclosure is directed to fusion proteins and related methods of use.

### BACKGROUND

DMD is a devastating, X-linked disorder that begins in the toddler years with difficulty with running, and progresses to loss of ambulation in the early teens followed by loss of arm, trunk, respiratory and cardiac muscle function. Death is common in early adulthood. DMD, and the milder allelic disorder, Becker muscular dystrophy (BMD), are caused by mutations in the dystrophin gene, which encodes an actin binding protein that links the actin cytoskeleton with the extracellular matrix, forming the dystrophin-associated glycoprotein complex (DGC) (Durbeej and Campbell, 2002; Ehmsen et al., 2002; Rando, 2001).

### SUMMARY OF THE INVENTION

Provided herein are compositions, including therapeutic and pharmaceutical compositions, comprising nNOSμ fusion proteins and derivatives thereof, and methods of treating neuromuscular disorders using such nNOSμ fusion proteins. As demonstrated herein, the inventors have discovered that nNOSμ is an important modulator of muscle function and that the loss of nNOSμ contributes to pathogenesis in neuromuscular diseases. The inventors have further discovered that sarcolemmal localization of nNOSμ significantly improves skeletal muscle pathology and restores muscle function in an accepted animal model of the neurodegenerative disorder muscular dystrophy.

Accordingly, provided herein in some aspects are therapeutic compositions comprising nNOSμ fusion proteins, wherein the nNOSμ fusion protein comprises an nNOSμ functional fragment and a plasma membrane targeting sequence.

In some embodiments of these aspects and all such aspects described herein, the plasma membrane targeting sequence is placed C-terminal to the nNOSμ functional fragment.

In some embodiments of these aspects and all such aspects described herein, the plasma membrane targeting sequence is a k-RAS palmitoylation signal sequence. In some embodiments of these aspects and all such aspects described herein, the k-RAS palmitoylation signal sequence consists of a sequence of SEQ ID NO: 6.

Also provided herein in some aspects are polypeptides comprising a palmitoylated nNOSμ polypeptide.

Provided herein in some aspects are vectors comprising a nucleotide sequence encoding: an nNOSμ fusion protein comprising an nNOSμ functional fragment and a plasma membrane targeting sequence; or a polypeptide comprising a palmitoylated nNOSμ polypeptide.

In some embodiments of these aspects and all such aspects described herein, the vector further comprises a nucleotide sequence encoding a muscle-specific promoter, enhancer, or both.

Provided herein in some aspects are modified RNA molecules comprising a nucleotide sequence encoding: an nNOSμ fusion protein comprising an nNOSμ functional fragment and a plasma membrane targeting sequence; or a polypeptide comprising a palmitoylated nNOSμ polypeptide, wherein said nucleotide sequence comprises at least two different modified nucleosides.

In some embodiments of these aspects and all such aspects described herein, the at least two different modified nucleosides are 5-methylcytidine and pseudouridine.

Provided herein in some aspects are methods of localizing an nNOSμ polypeptide to the sarcolemma comprising introducing: an nNOSμ fusion protein comprising an nNOSμ functional fragment and a plasma membrane targeting sequence; a vector comprising a nucleotide sequence encoding: an nNOSμ fusion protein comprising an nNOSμ functional fragment and a plasma membrane targeting sequence; or a polypeptide comprising a palmitoylated nNOSμ polypeptide; or a modified RNA molecule comprising a nucleotide sequence encoding: an nNOSμ fusion protein comprising an nNOSμ functional fragment and a plasma membrane targeting sequence; or peptides comprising peptides comprising a palmitoylated nNOSμ polypeptide, wherein said nucleotide sequence comprises at least two different modified nucleosides, to a muscle cell.

Also provided herein in some aspects are methods of localizing an nNOSμ polypeptide to the sarcolemma in a subject in need thereof comprising administering: an nNOSμ fusion protein comprising an nNOSμ functional fragment and a plasma membrane targeting sequence; a vector comprising a nucleotide sequence encoding: an nNOSμ fusion protein comprising an nNOSμ functional fragment and a plasma membrane targeting sequence; or a polypeptide comprising a palmitoylated nNOSμ polypeptide; or a modified RNA molecule comprising a nucleotide sequence encoding: an nNOSμ fusion protein comprising an nNOSμ functional fragment and a plasma membrane targeting sequence; or peptides comprising peptides comprising a palmitoylated nNOSμ polypeptide, wherein said nucleotide sequence comprises at least two different modified nucleosides to the subject in need thereof.

Also provided herein in some aspects are methods of treating a neuromuscular disease in a subject comprising administering: an nNOSμ fusion protein comprising an nNOSμ functional fragment and a plasma membrane targeting sequence; a vector comprising a nucleotide sequence encoding: an nNOSμ fusion protein comprising an nNOSμ functional fragment and a plasma membrane targeting sequence; or a polypeptide comprising a palmitoylated nNOSμ polypeptide; or a modified RNA molecule comprising a nucleotide sequence encoding: an nNOSμ fusion protein comprising an nNOSμ functional fragment and a plasma membrane targeting sequence; or peptides comprising peptides comprising a palmitoylated nNOSμ polypeptide, wherein said nucleotide sequence comprises at least two different modified nucleosides to the subject.

In some embodiments of these methods and all such methods described herein, the subject in need thereof has, or the neuromuscular disease is, one of: Duchenne muscular dystrophy, Becker muscular dystrophy, Limb-girdle muscular dystrophies, Ullrich congenital muscular dystrophy, inflammatory myositis, muscle atrophy, Amyotrophic lateral sclerosis, cachexia, and sarcopenia.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which the claimed invention belongs.

As used herein, the term “nNOSμ” polypeptide refers to a polypeptide translated from a mammalian nNOS gene sequence, or to a fragment or variant sequence thereof, that retains the ability to improve one or more of maximum Specific tetanic Force (SpF) at low frequency (physiological—as described in the Examples herein) stimulation, exercise-induced fatigue susceptibility, and susceptibility to stretch contraction-induced injury, when localized to the sarcolemma via fusion with a plasma membrane-targeting sequence. In this context, SpF, exercise-induced fatigue and stretch-contraction-induced injury can be measured, for example, as described in the Examples herein. Also in this context, an improvement is by at least 10% relative to the same cells without expression of membrane-targeted nNOSμ fusion. An nNOSμ polypeptide comprises muscle-specific exon sequence that distinguishes the μ isoform of nNOS from other variants generated by alternative splicing of the nNOS primary transcript in mammals. In humans, the muscle-specific exon sequence is YPEPLRFFPRKGPPLPNGDTEVHGLAAARDSQHR (SEQ ID NO: 5). In mice, the sequence is YPEPLRFFPRKGPSLSHADSEAHSLVAARDSQHR (SEQ ID NO: 52). Wild-type full length nNOSμ sequences include, for example, SEQ ID NO: 1 (human) and NP_032738.1 (mouse).

As used herein the term “nNOSμ fusion protein” refers to an nNOSμ polypeptide as described herein, fused with a plasma membrane targeting sequence, as described herein. By “fused” is meant that the nNOSμ polypeptide portion and plasma membrane targeting sequence are encoded bby and translated from a single open reading frame of an RNA molecule.

As used herein the term “nNOSμ protein derivative” or “nNOSμ fusion protein derivative,” depending on the context, refers to a protein or peptide that is derived from nNOSμ as described herein, i.e., a functional fragment or processed form of nNOSμ, and includes peptides or proteins which have been chemically modified by techniques such as adding additional side chains, ubiquitination, labeling, pegylation (derivatization with polyethylene glycol), and insertion, deletion or substitution of amino acids, including insertion, deletion and substitution of amino acids and other molecules (such as amino acid mimetics or unnatural amino acids) that do not normally occur in the amino acid sequence of nNOSμ.

In one embodiment, the nNOSμ portion of an nNOSμ fusion protein as described herein comprises no modifications or only conservative substitutions from among the 20 amino acids occurring in proteins naturally.

The term “functional fragment” as used in the context of a “functional fragment of nNOSμ” or “nNOSμ functional fragment thereof” refers to a fragment of nNOSμ polypeptide that improves or treats one or more symptoms or parameters of a neurodegenerative disorder such as, for example decreased maximum tetanic force, and/or increased susceptibility to fatigue when targeted to the sarcolemma via fusion with a membrane targeting sequence as described herein. Accordingly, the term “functional” when used in conjunction with a fragment, “derivative” or “variant” refers to a protein molecule that possesses a biological activity that is substantially similar to a biological activity of the entity or molecule of which it is a fragment, derivative or variant. By “substantially similar” in this context is meant that the biological activity, e.g., ability to increase skeletal muscle SpF, and/or decrease susceptibility to fatigue is at least 50% as active as a reference, e.g., a corresponding wild-type nNOSμ polypeptide targeted to the sarcolemma, and preferably at least 60% as active, 70% as active, 80% as active, 90% as active, 95% as active, 100% as active or even higher (i.e., the variant or derivative has greater activity than the wild-type), e.g., 110% as active, 120% as active, or more. Assays to measure the biological activity of an nNOSμ functional fragment are known in the art, and non-limiting examples are provided herein in the Examples.

As used herein, a “plasma membrane targeting sequence” refers to an amino acid sequence (or, depending on context, nucleotide sequence encoding the amino acid sequence), that permits or directs a protein, such as the nNOSμ fusion proteins described herein, to be targeted or localized to the plasma membrane of a cell, or sarcolemma of a muscle cell. The nNOSμ fusion proteins described herein can be tethered or otherwise attached to a cell membrane by various means, including utilizing known and natural sequences from membrane and/or cell surface proteins which are naturally attached to the cell membrane, or any such other means known or available in the art to attach or associate a peptide or molecule with the membrane or cell surface, including via attachment to or association with a membrane protein. Sequences from known and natural membrane and/or cell surface proteins serve to transverse or attach to the membrane per se or are recognized and function to signal attachment and/or specific modification in cells. Such sequences will be known and recognized to the skilled artisan and include for instance such sequences or domains as palmitoylation sites, myristoylation sites, transmembrane domains, and PH domains.

As used herein, the term “targeted to the sarcolemma” refers to a polypeptide that is directed for attachment to the sarcolemma by expression as a fusion with a membrane-targeting sequence.

As used herein, the term “protein” and “polypeptide” are used interchangeably. The terms “polypeptide” and “protein” are used interchangeably to refer to a polymer of linearly arranged amino acid residues linked by peptide bonds, whether produced biologically, recombinantly, or synthetically and whether composed of naturally occurring or non-naturally occurring amino acids. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, as used herein, a “polypeptide” refers to a protein that includes modifications, such as deletions, additions, and substitutions (generally conservative in nature, as would be known to a person skilled in the art) to the native sequence, as long as the protein maintains the desired functional activity. These modifications can be deliberate, as through site-directed mutagenesis, or can be accidental, such as through mutations of hosts that produce the proteins, or errors due to PCR amplification or other recombinant DNA methods.

The term “amino acid,” as used herein, is intended to refer to any natural or unnatural amino acid, whether made naturally or synthetically, including any such in L- or D-configuration. The term can include a modified or unusual amino acid or a synthetic derivative of an amino acid, e.g. diamino butyric acid and diamino propionic acid and the like. In the context of a polypeptide, an amino acid is synonymous with amino acid residue, as understood by one of ordinary skill in the art.

The terms “analog” or “variant” as used herein in reference to nNOSμ fusion proteins refers to a polypeptide or nucleic acid that differs from the naturally occurring polypeptide, i.e., nNOSμ or a domain thereof, or nucleic acid encoding such polypeptide, by one or more amino acid or nucleic acid deletions, additions, substitutions or side-chain modifications, yet retains one or more functions or biological activities (i.e., functions or activities specific to muscle function) of the naturally occurring molecule, i.e., nNOSμ. Amino acid substitutions include alterations in which an amino acid is replaced with a different naturally-occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Substitutions encompassed by variants as described herein may also be “non conservative,” in which an amino acid residue which is present in a peptide is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide).

As used herein the term “derivative” refers to a polypeptide that is derived from an wild-type nNOSμ as described herein, e.g., a fragment or processed form of nNOSμ, and includes peptides which have been chemically modified by techniques such as adding additional side chains, ubiquitination, labeling, pegylation (derivatization with polyethylene glycol), and insertion, deletion or substitution of amino acid mimetics and/or unnatural amino acids that do not normally occur in the sequence of wild-type nNOSμ that is basis of the derivative. For example, in some embodiments, the nNOSμ fusion protein derivatives can comprise a label, such as, for example, an epitope, e.g., a FLAG epitope or a V5 epitope or an HA epitope. Such a tag can be useful for, for example, purifying the fusion protein derivative. The term “derivative” also encompasses a derivatized polypeptide, such as, for example, a polypeptide modified to contain one or more-chemical moieties other than an amino acid. The chemical moiety can be linked covalently to the peptide, e.g., via an amino terminal amino acid residue, a carboxy terminal amino acid residue, or at an internal amino acid residue. Such modifications include the addition of a protective or capping group on a reactive moiety in the polypeptide, addition of a detectable label, and other changes that do not adversely destroy the activity of the nNOSμ fusion protein. In some embodiments, an nNOSμ derivative contains additional chemical moieties not normally a part of the molecule. Such moieties can improve its solubility, absorption, biological half life, etc. The moieties can alternatively decrease the toxicity of the molecule, or eliminate or attenuate an undesirable side effect of the molecule, etc. Moieties capable of mediating such effects are disclosed, for example, in Remington's Pharmaceutical Sciences, 18th edition, A. R. Gennaro, Ed., MackPubl., Easton, Pa. (1990).

The terms “decreased”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level (e.g., in the absence of an nNOSμ fusion protein described herein).

As used herein, the term “vector” refers to a nucleic acid molecule capable of transporting or mediating expression of a heterologous nucleic acid to which it has been linked, i.e., an nNOSμ fusion protein, to a host cell; a plasmid is a species of the genus encompassed by the term “vector.” The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. Expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome.

As used herein, the terms “synthetic, modified RNA” or “modified RNA” refer to an RNA molecule produced in vitro, which comprise at least one modified nucleoside as that term is defined herein below. The synthetic, modified RNA composition does not encompass mRNAs that are isolated from natural sources such as cells, tissue, organs etc., having those modifications, but rather only synthetic, modified RNAs that are synthesized using in vitro techniques.

As used herein the term “modified nucleoside” refers to a ribonucleoside that encompasses modification(s) relative to the standard guanine (G), adenine (A), cytidine (C), and uridine (U) nucleosides. Such modifications can include, for example, modifications normally introduced post-transcriptionally to mammalian cell mRNA, and artificial chemical modifications, as known to one of skill in the art.

The terms “subject” and “individual” are used interchangeably herein, and refer to an animal, for example a human recipient of the nNOSμ fusion proteins and derivatives described herein. For treatment of those neurodegenerative disorders that are specific for a specific animal, such as a human subject, the term “subject” refers to that specific animal. The terms ‘non-human animals’ and ‘non-human mammals’ are used interchangeably herein, and include, for example, mammals such as rats, mice, rabbits, sheep, cats, dogs, cows, pigs, and non-human primates.

The “therapeutically effective amount” of an nNOSμ fusion protein or derivative described herein is the minimum amount necessary to, for example, increase or improve one or more muscle function parameters, such as, for example, contractility, fatigue, and/or muscle damage, as assayed by methods known in the art and described herein. Accordingly, the “therapeutically effective amount” to be administered to a subject is governed by such considerations, and, as used herein, refers to the minimum amount necessary to prevent, ameliorate, treat, or stabilize, a neurodegenerative disorder or condition as described herein.

The practice of the methods described herein will employ, unless otherwise indicated, conventional techniques of molecular biology, cellular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Polynucleotide Hybridization (B. D. Harries & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All patents and other publications identified herein, both supra and infra, are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### BRIEF DESCRIPTION OF THE DRAWINGS

FIGS. 1A-1B show a schematic of nNOS isoforms. FIG. 1A. Scheme of alternative splicing of nNOS gene. Light and dark boxes represent noncoding and coding sequence, respectively. Asterisks represent start codons. FIG. 1B. The translated nNOS proteins are represented. PDZ scaffold domain, Protein Inhibitor of nNOS binding domain (PIN), Oxygenase domain, Binding site for Calcium Calmodulin (CaM), μ-insert, and Reductase domain.

FIG. 2 shows a schematic of the dystrophin-associated glycoprotein complex and nNOSμ. The DGC in skeletal muscle connects the actin cytoskeleton with the basal lamina. It is composed of dystrophin, dystroglycans (α, β), sarcoglycans (α, β, γ, δ), syntrophins (α, β1) and dystrobrevin (α). A growing number of proteins are reported to be associated with the DGC and have important roles in muscle function and disease, including nNOSμ. Several forms of muscular dystrophy arise from primary mutations in genes encoding components of the DGC. Mutations in dystrophin, all four sarcoglycans and the laminin α2 chain are responsible for DMD/BMD, LGMD type 2C-F and Congenital Muscular Dystrophy, respectively {Durbeej, 2002 #105; Ehmsen, 2002 #85; Rando, 2001 #88}.

FIGS. 3A-3D depict nNOSμ Transgenic Mice. FIG. 3A. Scheme of constructs expressed in transgenic mice. We expressed nNOS containing the μ exon, (the isoform normally expressed in skeletal muscle) with a C-terminal hemagglutinin tag (HA) or the HA tag followed by the palmitoylation signal sequence from the k-RAS oncogene. FIG. 3B. Western blot from Tibialis anterior (left) and Diaphragm (right) homogenates using an anti-NOS antibody. FIG. 3C. Western blot from muscle homogenate using an anti-HA antibody, detecting only the transgenic protein. FIG. 3D. Western blot from Diaphragm, Intestine, Liver, Brain, Heart, and TA muscle homogenates. Skeletal muscle and Brain show high expression of nNOS (p, and a, respectively). Anti-HA antibody shows only skeletal muscle expression of the transgene.

FIG. 4 demonstrates localization of nNOSμ-HA in C57Bl10 and mdx mice. 10 um sections of isopentane-frozen TA muscle were used for immunofluorescence. Staining against nNOS and the HA-tag were combined with antibody against caveolin and/or wheat germ agglutinin-488 (WGA) to visualize sarcolemma. nNOSμ can localize to the sarcolemma in the wt C57Bl10 mice (left). However, no sarcolemmal staining is seen when the transgene is expressed in the mdx background (right).

FIG. 5 demonstrates localization of nNOSμ-HA-RAS in C57Bl10 and mdx mice. 10 um sections of isopentane-frozen TA muscle were used for immunofluorescence. Staining against nNOS and the HA-tag were combined with antibody against caveolin to visualize sarcolemma. Transgenic nNOSμ-RAS protein shows sarcolemmal localization in both wt C57Bl10 and mdx backgrounds.

FIG. 6 demonstrates localization of nNOSμ in transgenic lines. Muscle cross sections from WT, mdx, mdx expressing nNOSμ and mdx expressing nNOSμ-RAS mice, were immunostained with an nNOS antibody. Note the sarcolemmal nNOS staining only in WT and mdx mice expressing nNOSμ-RAS.

FIGS. 7A-7D show ex vivo evaluation of Diaphragm Performance. Diaphragm strips were used to evaluate muscle function in transgenic mice expressing cytosolic nNOSμ and sarcolemmal localized nNOSμ-RAS. FIG. 7A. SpF (Maximal force normalized to Cross Sectional Area) vs Frequency of stimulation curve. FIG. 7B. Twitch SpF, SpF evoked for a single stimulus, was measured in diaphragm. FIGS. 7C-7D. Diaphragm strips were subjected to a fatigue protocol, consisting of isometric tetanic contractions (120 Hz) every 1 second for a total of 1 minute. Force values (mean±SEM) are shown as percentage of Initial force. Pooled data for mice in C57Bl10 (FIG. 7C) or mdx (FIG. 7D) background.

FIGS. 8A-8D show in situ evaluation of Tibialis anterior Performance. Whole TA muscle in its natural environment, keeping the anesthetized mouse alive while the experiment was performed, was used to evaluate muscle function in transgenic mice expressing cytosolic nNOSμ and sarcolemmal localized nNOSμ-RAS. FIG. 8A. SpF vs Frequency of stimulation curve. FIG. 8B. Twitch SpF, SpF evoked for a single stimulus, was measured in TA. FIGS. 8C-8D. TA was subjected to the fatigue protocol consisting of isometric tetanic contractions (200 Hz) every 2 seconds for a total of 4 minutes. Force values (mean±SEM) are percentage of Initial force, and only values every 10 seconds are shown for clarity. Pooled data for mice in C57Bl10 (FIG. 8C) or mdx (FIG. 8D) background.

FIGS. 9A-9B show in situ evaluation of ECC-induced damage. FIG. 9A. Scheme of ECC protocol. A tetanic stimulus (200 Hz) of 350 ms of duration. First, muscle undergoes an isometric contraction. Then, muscle is stretched to 20% of its L0, at a velocity of 2 L0 per second, where it is held until the end of the stimulus. Consecutive stretching causes a decrease in the maximal isometric force of the following contraction. 10 consecutive ECCs are performed every 1 minute. FIG. 9B. TA muscles underwent 10 Eccentric contractions in situ and isometric force was measured during each contraction. Pooled data (mean±SEM) to compare C57Bl10, mdx and mdx expressing nNOSμ and nNOSμ-RAS mice.

FIGS. 10A-10D demonstrate centronucleation and fiber area variation in Dystrophic Muscle. FIG. 10A. Fluorescent images showing WGA-488 and DAPI staining to observe the fiber membrane and nuclei in diaphragm (top) and TA (bottom). Half of the diaphragm or whole TA were analyzed, using a grid to evaluate fibers covering the full area stained. FIG. 10B. Percentage of central nuclei in diaphragm from mdx and transgenic mice in the mdx background. FIG. 10C. Percentage of central nuclei in TA from mdx and transgenic mice on the mdx background. Values are mean±SEM. 1 way Anova with Dunnet's multicomparison test: ** p≦0.01. FIG. 10D. Cumulative Frequency of TA fiber area values for mdx and mdx transgenic mice.

FIGS. 11A-11D show fibronectin and Collagen I staining in Diaphragm. FIG. 11A. Immunofluorescence for Fibronectin (left) and Collagen I (right). A portion of the diaphragm is shown. FIG. 11B. Quantification of the percentage of area occupied by fibronectin in diaphragm from mdx and transgenic mice in the mdx background. FIG. 11C. Quantification of the percentage of area occupied by Collagen I in diaphragm from mdx and transgenic mice in the mdx background. Values are mean±SEM. FIG. 11D. Western blot against Fibronectin on Diaphragm homogenates. Transgenic mice expressing nNOS pi and nNOSμ-RAS are compared to non-transgenic littermates.

FIGS. 12A-12D show fibronectin and Collagen I staining in TA muscle. FIG. 12A. Immunofluorescence for Fibronectin (left) and Collagen I (right). A portion of the TA is shown, but whole TA was quantified. FIG. 12B. Quantification of the percentage of area occupied by fibronectin in TA from mdx and transgenic mice in the mdx background. FIG. 12C. Quantification of the percentage of area occupied by Collagen I in TA from mdx and transgenic mice in the mdx background. Values are mean±SEM. FIG. 12D. Western blot pf Fibronectin on TA homogenates. Transgenic mice expressing nNOSμ and nNOSμ-RAS are compared to non-transgenic littermates.

FIG. 13 shows that in sarcolemmal localized nNOSμ, α-dystrobrevin colocalizes with nNOSμ-RAS on the sarcolemma. Sarcolemmal localization of nNOSμ or dystrobrevin was not observed on transgenic mice expressing nNOSμ.

FIGS. 14A-14I demonstrate generation and characterization of transgenic mice.

FIG. 15 demonstrates that sarcolemmal nNOS reduces eccentric contraction damage in TA muscle.

FIG. 16 demonstrates that sarcolemmal nNOSμ reduces fatigue in TA muscle.

FIG. 17 demonstrates diaphragm fatigue.

FIGS. 18A-18H demonstrate that mice expressing sarcolemmal nNOSμ had significant increases in many of the dystrophin complex proteins, including utrophin, dystrobrevin, syntrophin, beta-dystroglycan, and aquaporin-4.

### DETAILED DESCRIPTION

As described herein, the impact of restoring the expression and localization of the skeletal muscle isoform nNOSμ on dystrophic pathology or muscle function was tested, as was the role of the μ-domain in skeletal muscle nNOSμ. It is demonstrated herein that nNOSμ is an important modulator of muscle function, and that the loss of nNOSμ contributes to pathogenesis in neuromuscular diseases, including DMD. Furthermore, the impact of the restoration of cytoplasmic and sarcolemma-localized nNOSμ on mdx skeletal muscle pathology and function is demonstrated.

### Neuronal Nitric Oxide Synthase

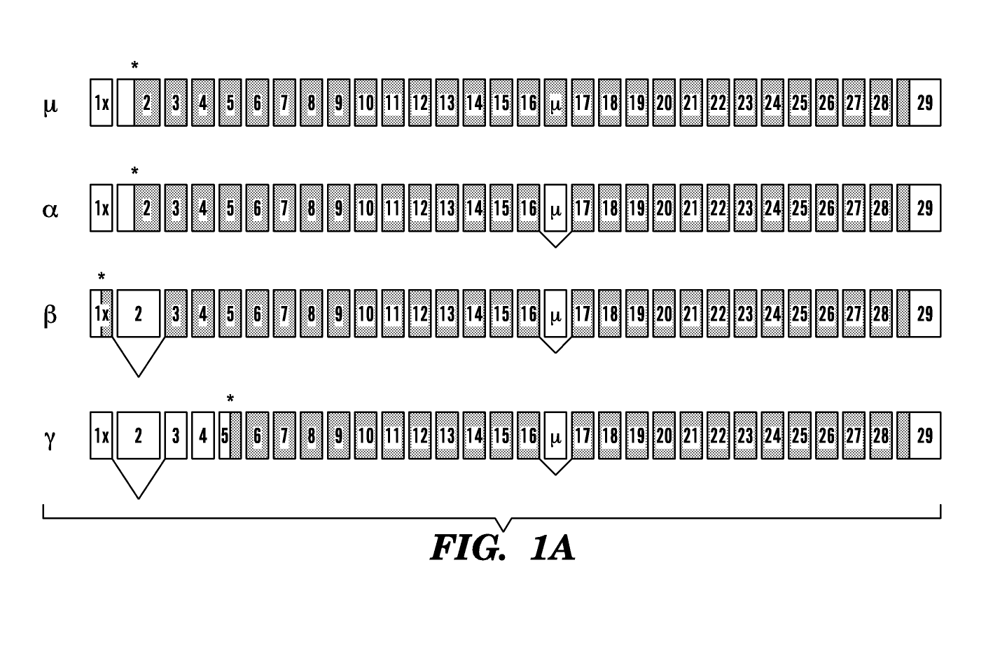

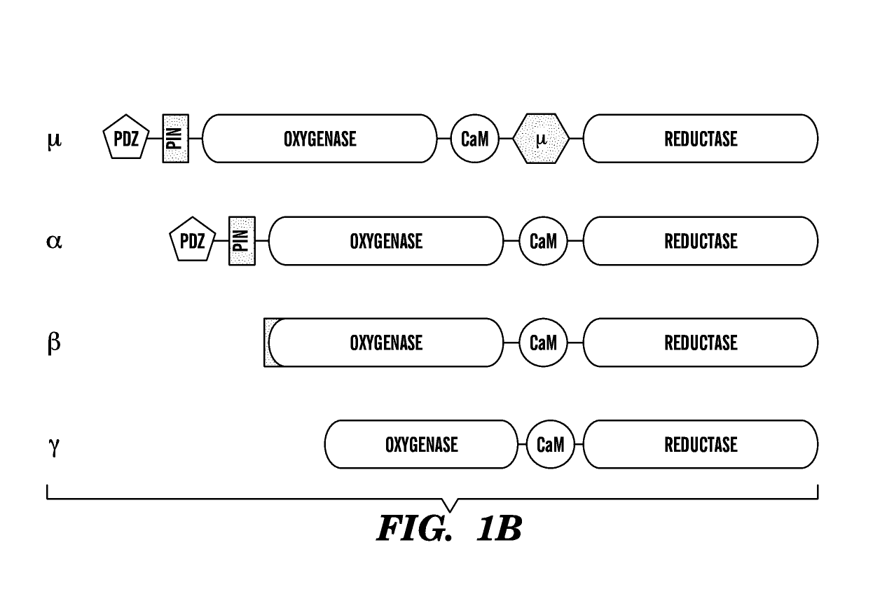

Neuronal nitric oxide synthase (nNOS) is a constitutively expressed Ca²⁺/calmodulin-regulated protein that catalyzes the synthesis of nitric oxide (NO) from L-arginine. The nNOS protein consists of 2 functional domains: an N-terminal oxygenase domain and a C-terminal reductase domain separated by a Ca²⁺/calmodulin binding site (FIGS. 1A-1B and (Zhou and Zhu, 2009)). Alternative splicing of the product of the single nNOS gene leads to the production of different isoforms: nNOSα, nNOSβ, nNOSγ and nNOSμ (Wang et al., 1999).

nNOSα is mainly expressed in brain, in neurons and astrocytes (Zhou and Zhu, 2009). A PDZ domain in the N-terminus of the protein is able to interact with other proteins containing PDZ domains, such as proteins of the postsynaptic density, targeting nNOSα to the plasma membrane (Brenman et al., 1996; Zhou and Zhu, 2009).

nNOSβ and nNOSγ are generated by excluding exon 2 and employing different first exons. Both isoforms lack the PDZ targeting domain and the PIN (Protein Inhibitor of nNOS) binding domain (FIGS. 1A-1B), which is encoded by exon 2; therefore, neither nNOSβ or nNOSγ isoform is localized to the plasma membrane. In vitro assays have shown that nNOSγ has no significant catalytic activity, while nNOSβ has an activity similar to nNOSα (Brenman et al., 1996; Eliasson et al., 1997).

nNOSμ has a 102-base pair exon between exons 16 and 17, corresponding to a 34 amino acid insert between the calmodulin and FMN (flavin mononucleotide) binding site of the reductase domain. It is specifically expressed in skeletal and cardiac muscles, and its in vitro activity as well as its regulation by calcium and calmodulin has been reported to be very similar to nNOSα (Silvagno et al., 1996). However, these parameters have been measured only in vitro with the purified proteins (Silvagno et al., 1996), and the supposed functional equivalence of both isoforms remains untested. The function of the μ-domain is unknown, but has been postulated to be involved in membrane association and possible posttranslational regulation of nNOSμ (Larsson and Phillips, 1998; Silvagno et al., 1996). For instance, because the rodent μ-domain contains 5 serine residues, it has been proposed as a possible site of regulation by phosphorylation (Silvagno et al., 1996). Interaction with proteins, different or in addition to those interacting with nNOSα, could also be another form of regulation of nNOSμ.

nNOSμ and nNOSβ are both coexpressed in skeletal muscle cells and both generate NO. NO signals through 2 different pathways. 1) In the cGMP-dependent pathway, NO binds to soluble guanylyl cyclase (sGC), the so-called “NO-receptor”, stimulating the conversion of GTP into cGMP. This second messenger binds to downstream effectors that include cGMP-dependent protein kinase (PKG), cGMP-gated channels, and cGMP-regulated phosphodiesterases (Bender and Beavo, 2006; Craven and Zagotta, 2006; Hofmann et al., 2009). 2) In the cGMP-independent pathway, NO can directly react with thiol residues of cysteines (S-nitrosylation) in some proteins, modifying their activity. For instance, the ryanodine (RyR1) Ca²⁺ release channel is activated by S-nitrosylation (Eu et al., 2000).

Although both nNOSμ and nNOSβ splice variants are expressed in muscle cells, they have different subcellular localizations. Recently, nNOSβ was shown to target to the Golgi complex in murine skeletal muscle, where it acts as a critical regulator of muscle structural and functional integrity (Percival et al., 2010). On the other hand, nNOSμ is present in the cytoplasm and is also localized to the inner surface of the sarcolemma, by binding the dystrophin-associated glycoprotein complex (DGC) member, α-syntrophin (Brenman et al., 1995; Miyagoe-Suzuki and Takeda, 2001).

The roles of nNOSμ in skeletal muscle include regulation of fatigue resistance, maintenance of blood delivery during exercise, control of muscle mass, and modulation of glucose homeostasis (Percival et al., 2008; Ross et al., 2007; Thomas et al., 1998; Thomas et al., 2003; Wehling-Henricks et al., 2009). The sarcolemmal localization is critical for nNOSμ to oppose sympathetic vasoconstriction and maintain appropriate blood supply to active muscles, including during exercise (Thomas et al., 2003). Furthermore, it has been proposed that nNOSμ plays a role in regulating neuromuscular junction (NMJ) structure by increasing the expression and clustering of nicotinic acetylcholine receptors (Shiao et al., 2004) and can regulate phosphofructokinase (PFK) activity, the rate-limiting enzyme in glycolysis (Wehling-Henricks et al., 2009). In normal muscle, nNOSμ localizes mainly to the cytosolic side of the sarcolemma, by binding the dystrophin-associated glycoprotein complex (DGC) member α-syntrophin. nNOSμ also regulates cardiac, skeletal and smooth muscle contractile function and modulates the immune response in muscle.

nNOSμ expression, localization and/or signaling are impaired in many neuromuscular diseases of diverse genetic etiology, including Duchenne muscular dystrophy (DMD) (Brenman et al., 1995), Becker muscular dystrophy (Chao et al., 1996), Limb-girdle muscular dystrophies (LGMD) 2C, 2D and 2E (Crosbie et al., 2002), Ullrich congenital muscular dystrophy, inflammatory myositis (Kobayashi et al., 2008), muscle atrophy and Amyotrophic lateral sclerosis (ALS) (Suzuki et al., 2010; Suzuki et al., 2007). Furthermore, nNOSμ deficiency can contribute to the muscle fatigue that is a common and poorly understood characteristic of many of these diseases, including DMD (Kobayashi et al., 2008; Percival et al., 2010; Schillings et al., 2007). Moreover, myopathic deficits in nNOSμ-deficient skeletal muscle, such as reduced skeletal muscle mass, decreased maximum tetanic force and increased susceptibility to fatigue, indicate that loss of nNOSμ may be contributing to disease severity (Percival et al., 2008). Understanding the molecular mechanisms of muscle fatigue also has relevance beyond DMD and neuromuscular disease to cardiovascular disease and diabetes characterized by exaggerated skeletal muscle fatigue.

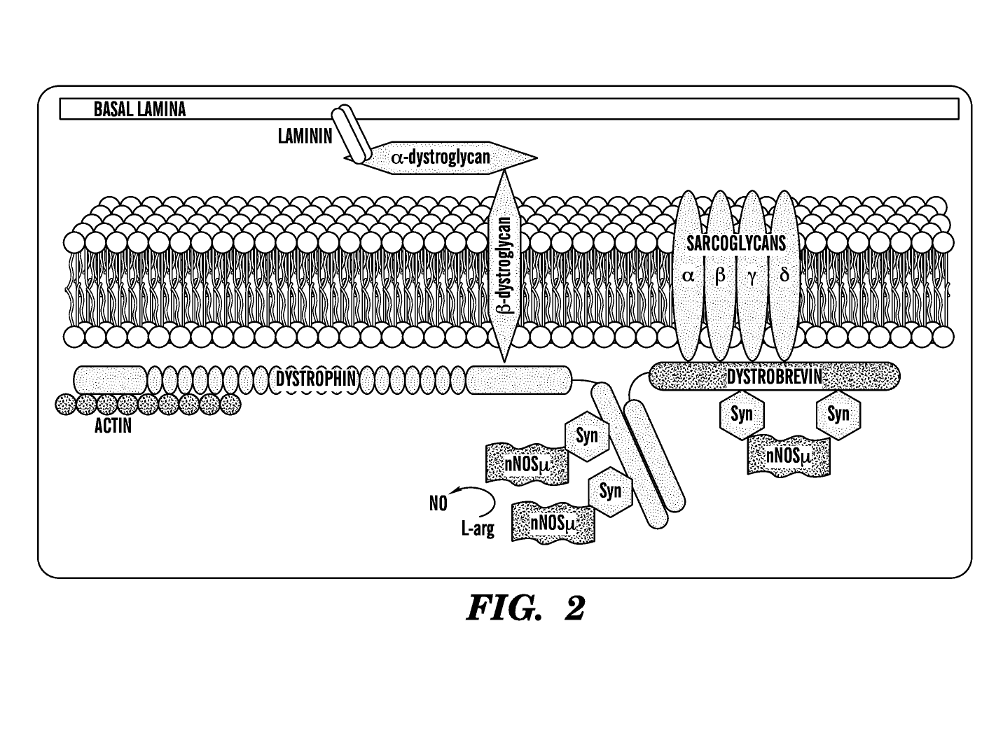

DMD is a devastating, X-linked disorder that begins in the toddler years with difficulty with running and progresses to loss of ambulation in the early teens followed by loss of arm, trunk, respiratory and cardiac muscle function. Death is common in early adulthood. DMD, and the milder allelic disorder, Becker muscular dystrophy (BMD), are caused by mutations in the dystrophin gene, which encodes an actin binding protein that links the actin cytoskeleton with the extracellular matrix, forming the dystrophin-associated glycoprotein complex (DGC) (FIG. 2) (Durbeej and Campbell, 2002; Ehmsen et al., 2002; Rando, 2001). The dystrophin deficiency leads to loss of other DGC members, including nNOSμ (Ibraghimov-Beskrovnaya et al., 1992; Waite et al., 2009). In both DMD patients and the mdx mouse model for DMD, nNOSμ expression and activity is greatly decreased and residual nNOSμ fails to localize to the sarcolemma (Brenman et al., 1995; Chang et al., 1996). In DMD and other muscular dystrophies, there is an urgent, critical need for efficacious and safe treatments that slow disease progression. Such therapies will reduce disease burden, improve quality of life and buy time until treatments that address the primary gene defect are fully developed, affordable, and widely available.

Transgenic expression of nNOS in the mdx mouse has been reported to improve the histopathology of skeletal muscle, without targeting of the enzyme to the sarcolemma. The cytoplasmic expression of a rat brain nNOSα transgene reduced muscle membrane damage and inflammation in mdx muscle (Wehling et al., 2001). They propose an anti-inflammatory function for nNOS where NO protects muscle fibers from damage caused by macrophages (Wehling et al., 2001) plus a role for nNOS in positively regulating glycolytic metabolism through allosteric modulation of PFK, which could decrease fatigability of mdx mice (Wehling-Henricks et al., 2009).

While these results are consistent with a positive impact of nNOS over expression on the dystrophic histopathology, these previous approaches had several limitations. First, as mentioned before, these studies used nNOSα, the brain isoform rather than the muscle-specific nNOSμ. Second, the enzyme was expressed in skeletal muscle at very high levels (50-250 times normal) (Tidball and Wehling-Henricks, 2004). In addition, the high levels of nNOS in the mdx context resided entirely in the cytosol (or at least, not on the sarcolemma), due to the absence of dystrophin and the DGC. Thus, the effects observed likely do not correspond to the physiological role of the skeletal muscular isoform nNOSμ in its correct localization. Furthermore, this issue becomes important because a toxic gain of function has been suggested for nNOSμ in non-dystrophic muscle when the enzyme is not localized to the sarcolemma. α1-Syntrophin-null muscles show displacement of nNOSμ from the sarcolemma and do not regenerate normally (Hosaka et al., 2002), while in tail-suspension, denervation and Amyotrophic Lateral Sclerosis (ALS) models, nNOSμ has been shown to be mislocalized and induce muscle atrophy through the activation of FoxO3a and muscle-specific E3 ubiquitin ligases MuRF-1 and atrogin-1/MAFbx (Suzuki et al., 2010; Suzuki et al., 2007). Finally, no contractile physiological studies were performed to determine if nNOSα expression had a functional effect, positive or negative.

nNOSμ Fusion Proteins and Derivatives Thereof

In one aspect, the present disclosure is directed to fusion proteins comprising nNOSμ polypeptides and a plasma membrane targeting sequence. In certain further embodiments, the plasma membrane targeting sequence is a k-RAS palmitoylation signal sequence.

Provided herein, in some aspects, are compositions, including, but not limited to, therapeutic and pharmaceutical compositions comprising nNOSμ fusion proteins and derivatives thereof comprising a functional fragment of nNOSμ and a plasma membrane targeting sequence. Such nNOSμ fusion proteins and derivatives thereof can be used to improve or treat one or parameters or symptoms of a neurodegenerative disorder, such as, reduced skeletal muscle mass, decreased maximum tetanic force, and/or increased susceptibility to fatigue.

nNOSμ is a skeletal and cardiac muscle-specific isoform of nNOS and has a 102-base pair exon between exons 16 and 17, corresponding to a 34 amino acid insert between the calmodulin and FMN (flavin mononucleotide) binding site of the reductase domain. nNOSμ is present in the cytoplasm and in normal muscle, nNOSμ localizes mainly to the cytosolic side of the sarcolemma, by binding the dystrophin-associated glycoprotein complex (DGC) member α-syntrophin (Brenman et al., 1995; Miyagoe-Suzuki and Takeda, 2001).

As used herein, “nNOSμ” refers to the 1468 amino acid human polypeptide having the sequence of:

(SEQ ID NO: 1)
MEDHMFGVQQIQPNVISVRLFKRKVGGLGFLVKERVSKPPVIISDL
IRGGAAEQSGLIQAGDIILAVNGRPLVDLSYDSALEVLRGIASETH
VVLILRGPEGFTTHLETTFTGDGTPKTIRVTQPLGPPTKAVDLSHQ
PPAGKEQPLAVDGASGPGNGPQHAYDDGQEAGSLPHANGLAPRPPG
QDPAKKATRVSLQGRGENNELLKEIEPVLSLLTSGSRGVKGGAPAK
AEMKDMGIQVDRDLDGKSHKPLPLGVENDRVFNDLWGKGNVPVVLN
NPYSEKEQPPTSGKQSPTKNGSPSKCPRFLKVKNWETEVVLTDTLH
LKSTLETGCTEYICMGSIMHPSQHARRPEDVRTKGQLFPLAKEFID
QYYSSIKRFGSKAHMERLEEVNKEIDTTSTYQLKDTELIYGAKHAW
RNASRCVGRIQWSKLQVFDARDCTTAHGMFNYICNHVKYATNKGNL
RSAITIFPQRTDGKHDFRVWNSQLIRYAGYKQPDGSTLGDPANVQF
TEICIQQGWKPPRGRFDVLPLLLQANGNDPELFQIPPELVLEVPIR
HPKFEWFKDLGLKWYGLPAVSNMLLEIGGLEFSACPFSGWYMGTEI
GVRDYCDNSRYNILEEVAKKMNLDMRKTSSLWKDQALVEINIAVLY
SFQSDKVTIVDHHSATESFIKHMENEYRCRGGCPADWVWIVPPMSG
SITPVFHQEMLNYRLTPSFEYQPDPWNTHVWKGTNGTPTKRRAIGF
KKLAEAVKFSAKLMGQAMAKRVKATILYATETGKSQAYAKTLCEIF
KHAFDAKVMSMEEYDIVHLEHETLVLVVTSTFGNGDPPENGEKFGC
ALMEMRHPNSVQEERKYPEPLRFFPRKGPPLPNGDTEVHGLAAARD
SQHRSYKVRFNSVSSYSDSQKSSGDGPDLRDNFESAGPLANVRFSV
FGLGSRAYPHFCAFGHAVDTLLEELGGERILKMREGDELCGQEEAF
RTWAKKVFKAACDVFCVGDDVNIEKANNSLISNDRSWKRNKFRLTF
VAEAPELTQGLSNVHKKRVSAARLLSRQNLQSPKSSRSTIFVRLHT
NGSQELQYQPGDHLGVFPGNHEDLVNALIERLEDAPPVNQMVKVEL
LEERNTALGVISNWTDELRLPPCTIFQAFKYYLDITTPPTPLQLQQ
FASLATSEKEKQRLLVLSKGLQEYEEWKWGKNPTIVEVLEEFPSIQ
MPATLLLTQLSLLQPRYYSISSSPDMYPDEVHLTVAIVSYRTRDGE
GPIHHGVCSSWLNRIQADELVPCFVRGAPSFHLPRNPQVPCILVGP
GTGIAPFRSFWQQRQFDIQHKGMNPCPMVLVFGCRQSKIDHIYREE
TLQAKNKGVFRELYTAYSREPDKPKKYVQDILQEQLAESVYRALKE
QGGHIYVCGDVTMAADVLKAIQRIMTQQGKLSAEDAGVFISRMRDD
NRYHEDIFGVTLRTYEVTNRLRSESIAFIEESKKDTDEVFSS,

as described by, e.g., NP_{—} 001191147.1, and encoded by, e.g., the polynucleotide sequence:

| (SEQ ID NO: 2; e.g., NM_001204218) | |
|---|---|
| ataaaagatg tatgctttgg agcccagagc ggctctttta atgagggttg cgacgtctcc | 61 |
| ctccccacac ccataaacca gtcgggttgg acgtcactgc taattcgttt cagtgatgat | 121 |
| aggataaagg agggacatta agaaataaat tccccctcac gaccctcgct gagctcacgg | 181 |
| ctcagtccct acatatttat gccgcgtttc cagccgctgg gtgaggagct acttagcgcc | 241 |
| gcggctcctc cgaggggcgg ccgggcagcg agcagcggcc gagcggacgg gctcatgatg | 301 |
| cctcagatct gatccgcatc taacaggctg gcaatgaaga tacccagaga atagttcaca | 361 |
| tctatcatgc gtcacttcta gacacagcca tcagacgcat ctcctcccct ttctgcctga | 421 |
| ccttagggac acgtcccacc gcctctcttg acgtctgcct ggtcaaccat cacttcctta | 481 |
| gagaataagg agagaggcgg atgcaggaaa tcatgccacc gacgggccac cagccatgag | 541 |
| tgggtgacgc tgagctgacg tcaaagacag agagggctga agccttgtca gcacctgtca | 601 |
| ccccggctcc tgctctccgt gtagcctgaa gcctggatcc tcctggtgaa atcatcttgg | 661 |
| cctgatagca ttgtgaggtc ttcagacagg acccctcgga agctagttac catggaggat | 721 |
| cacatgttcg gtgttcagca aatccagccc aatgtcattt ctgttcgtct cttcaagcgc | 781 |
| aaagttgggg gcctgggatt tctggtgaag gagcgggtca gtaagccgcc cgtgatcatc | 841 |
| tctgacctga ttcgtggggg cgccgcagag cagagtggcc tcatccaggc cggagacatc | 901 |
| attcttgcgg tcaacggccg gcccttggtg gacctgagct atgacagcgc cctggaggta | 961 |
| ctcagaggca ttgcctctga gacccacgtg gtcctcattc tgaggggccc tgaaggtttc | 1021 |
| accacgcacc tggagaccac ctttacaggt gatgggaccc ccaagaccat ccgggtgaca | 1081 |
| cagcccctgg gtccccccac caaagccgtg gatctgtccc accagccacc ggccggcaaa | 1141 |
| gaacagcccc tggcagtgga tggggcctcg ggtcccggga atgggcctca gcatgcctac | 1201 |
| gatgatgggc aggaggctgg ctcactcccc catgccaacg gcctggcccc caggccccca | 1261 |
| ggccaggacc ccgcgaagaa agcaaccaga gtcagcctcc aaggcagagg ggagaacaat | 1321 |
| gaactgctca aggagataga gcctgtgctg agccttctca ccagtgggag cagaggggtc | 1381 |
| aagggagggg cacctgccaa ggcagagatg aaagatatgg gaatccaggt ggacagagat | 1441 |
| ttggacggca agtcacacaa acctctgccc ctcggcgtgg agaacgaccg agtcttcaat | 1501 |
| gacctatggg ggaagggcaa tgtgcctgtc gtcctcaaca acccatattc agagaaggag | 1561 |
| cagcccccca cctcaggaaa acagtccccc acaaagaatg gcagcccctc caagtgtcca | 1621 |
| cgcttcctca aggtcaagaa ctgggagact gaggtggttc tcactgacac cctccacctt | 1681 |
| aagagcacat tggaaacggg atgcactgag tacatctgca tgggctccat catgcatcct | 1741 |
| tctcagcatg caaggaggcc tgaagacgtc cgcacaaaag gacagctctt ccctctcgcc | 1801 |
| aaagagttta ttgatcaata ctattcatca attaaaagat ttggctccaa agcccacatg | 1861 |
| gaaaggctgg aagaggtgaa caaagagatc gacaccacta gcacttacca gctcaaggac | 1921 |
| acagagctca tctatggggc caagcacgcc tggcggaatg cctcgcgctg tgtgggcagg | 1981 |
| atccagtggt ccaagctgca ggtattcgat gcccgtgact gcaccacggc ccacgggatg | 2041 |
| ttcaactaca tctgtaacca tgtcaagtat gccaccaaca aagggaacct caggtctgcc | 2101 |
| atcaccatat tcccccagag gacagacggc aagcacgact tccgagtctg gaactcccag | 2161 |
| ctcatccgct acgctggcta caagcagcct gacggctcca ccctggggga cccagccaat | 2221 |
| gtgcagttca cagagatatg catacagcag ggctggaaac cgcctagagg ccgcttcgat | 2281 |
| gtcctgccgc tcctgcttca ggccaacggc aatgaccctg agctcttcca gattcctcca | 2341 |
| gagctggtgt tggaagttcc catcaggcac cccaagtttg agtggttcaa ggacctgggg | 2401 |
| ctgaagtggt acggcctccc cgccgtgtcc aacatgctcc tagagattgg cggcctggag | 2461 |
| ttcagcgcct gtcccttcag tggctggtac atgggcacag agattggtgt ccgcgactac | 2521 |
| tgtgacaact cccgctacaa tatcctggag gaagtggcca agaagatgaa cttagacatg | 2581 |
| aggaagacgt cctccctgtg gaaggaccag gcgctggtgg agatcaatat cgcggttctc | 2641 |
| tatagcttcc agagtgacaa agtgaccatt gttgaccatc actccgccac cgagtccttc | 2701 |
| attaagcaca tggagaatga gtaccgctgc cgggggggct gccctgccga ctgggtgtgg | 2761 |
| atcgtgcccc ccatgtccgg aagcatcacc cctgtgttcc accaggagat gctcaactac | 2821 |
| cggctcaccc cctccttcga ataccagcct gatccctgga acacgcatgt ctggaaaggc | 2881 |
| accaacggga cccccacaaa gcggcgagcc attggcttca agaagctagc agaagctgtc | 2941 |
| aagttctcgg ccaagctgat ggggcaggct atggccaaga gggtgaaagc gaccatcctc | 3001 |
| tatgccacag agacaggcaa atcgcaagct tatgccaaga ccttgtgtga gatcttcaaa | 3061 |
| cacgcctttg atgccaaggt gatgtccatg gaagaatatg acattgtgca cctggaacat | 3121 |
| gaaactctgg tccttgtggt caccagcacc tttggcaatg gagatccccc tgagaatggg | 3181 |
| gagaaattcg gctgtgcttt gatggaaatg aggcacccca actctgtgca ggaagaaagg | 3241 |
| aagtacccgg aacccttgcg tttctttccc cgtaaagggc ctcccctccc caatggtgac | 3301 |
| acagaagtcc acggtctggc tgcagcccgt gacagccagc acaggagcta caaggtccga | 3361 |
| ttcaacagcg tctcctccta ctctgactcc caaaaatcat caggcgatgg gcccgacctc | 3421 |
| agagacaact ttgagagtgc tggacccctg gccaatgtga ggttctcagt ttttggcctc | 3481 |
| ggctcacgag cataccctca cttttgcgcc ttcggacacg ctgtggacac cctcctggaa | 3541 |
| gaactgggag gggagaggat cctgaagatg agggaagggg atgagctctg tgggcaggaa | 3601 |
| gaggctttca ggacctgggc caagaaggtc ttcaaggcag cctgtgatgt cttctgtgtg | 3661 |
| ggagatgatg tcaacattga aaaggccaac aattccctca tcagcaatga tcgcagctgg | 3721 |
| aagagaaaca agttccgcct cacctttgtg gccgaagctc cagaactcac acaaggtcta | 3781 |
| tccaatgtcc acaaaaagcg agtctcagct gcccggctcc ttagccgtca aaacctccag | 3841 |
| agccctaaat ccagtcggtc aactatcttc gtgcgtctcc acaccaacgg gagccaggag | 3901 |
| ctgcagtacc agcctgggga ccacctgggt gtcttccctg gcaaccacga ggacctcgtg | 3961 |
| aatgccctga tcgagcggct ggaggacgcg ccgcctgtca accagatggt gaaagtggaa | 4021 |
| ctgctggagg agcggaacac ggctttaggt gtcatcagta actggacaga cgagctccgc | 4081 |
| ctcccgccct gcaccatctt ccaggccttc aagtactacc tggacatcac cacgccacca | 4141 |
| acgcctctgc agctgcagca gtttgcctcc ctagctacca gcgagaagga gaagcagcgt | 4201 |
| ctgctggtcc tcagcaaggg tttgcaggag tacgaggaat ggaaatgggg caagaacccc | 4261 |
| accatcgtgg aggtgctgga ggagttccca tctatccaga tgccggccac cctgctcctg | 4321 |
| acccagctgt ccctgctgca gccccgctac tattccatca gctcctcccc agacatgtac | 4381 |
| cctgatgaag tgcacctcac tgtggccatc gtttcctacc gcactcgaga tggagaagga | 4441 |
| ccaattcacc acggcgtatg ctcctcctgg ctcaaccgga tacaggctga cgaactggtc | 4501 |
| ccctgtttcg tgagaggagc acccagcttc cacctgcccc ggaaccccca agtcccctgc | 4561 |
| atcctcgttg gaccaggcac cggcattgcc cctttccgaa gcttctggca acagcggcaa | 4621 |
| tttgatatcc aacacaaagg aatgaacccc tgccccatgg tcctggtctt cgggtgccgg | 4681 |
| caatccaaga tagatcatat ctacagggaa gagaccctgc aggccaagaa caagggggtc | 4741 |
| ttcagagagc tgtacacggc ttactcccgg gagccagaca aaccaaagaa gtacgtgcag | 4801 |
| gacatcctgc aggagcagct ggcggagtct gtgtaccgag ccctgaagga gcaagggggc | 4861 |
| cacatatacg tctgtgggga cgtcaccatg gctgctgatg tcctcaaagc catccagcgc | 4921 |
| atcatgaccc agcaggggaa gctctcggca gaggacgccg gcgtattcat cagccggatg | 4981 |
| agggatgaca accgatacca tgaggatatt tttggagtca ccctgcgaac gtacgaagtg | 5041 |
| accaaccgcc ttagatctga gtccattgcc ttcattgaag agagcaaaaa agacaccgat | 5101 |
| gaggttttca gctcctaact ggaccctctt gcccagccgg ctgcaagttt tgtaagcgcg | 5161 |
| gacagacact gctgaacctt tcctctggga ccccctgtgg ccctcgctct gcctcctgtc | 5221 |
| cttgtcgctg tgccctggtt tccctcctcg ggcttctcgc ccctcagtgg tttcctcggc | 5281 |
| cctcctgggt ttactccttg agttttcctg ctgcgatgca atgcttttct aatctgcagt | 5341 |
| ggctcttaca aaactctgtt cccactccct ctcttgccga caagggcaac tcacgggtgc | 5401 |
| atgaaaccac tggaacatgg ccgtcgctgt gggggttttt ttctctgggg ttcccctgga | 5461 |
| aaggctgcag gaactaggca caagctctct gagccagtcc ctcagccact gaagtccccc | 5521 |
| tttctccttt tttatgatga cattttggtt gtgcgtgcct gtgtgtgtgt gtgtgtgtgt | 5581 |
| gtgtgtgtgt gtgatgggcc aggtctctgt ccgtcctctt ccctgcacaa gtgtgtcgat | 5641 |
| cttagattgc cactgctttc attgaagacc ctcaatgcca agaaacgtgt ccctggccca | 5701 |
| tattaatccc tcgtgtgtcc ataattaggg tccacgccca tgtacctgaa acatttggaa | 5761 |
| gccccataat tgttctagtt agaaagggtt cagggcatgg ggagaggagt gggaaattga | 5821 |
| ttaaaggggc tgtctcccaa tgaaagaggc attcccagaa tttgctgcat ttagattttg | 5881 |
| ataccagtga gcagagccct catgtgacat gaacccatcc aatggattgt gcaaatcccc | 5941 |
| tccccaaacc cacccatacc agctagaatc acttgacttt gccacatcca ttgactgacc | 6001 |
| ccctcctcca gcaatagcat ccaaggggcc tggaagttat gttgttcaaa gaagcctggt | 6061 |
| ggcaataagg atcttcccac tttgccactg gatgactttg gatgggtcac ttgtcctcag | 6121 |
| tttttcctag tcataatgtc atacgaacct aaagaatatg aatggattaa atgttaaagc | 6181 |
| tttggtgcct ggaaacaata tcaagtaaca atatgattat tattttttta ttcccccaaa | 6241 |
| gcgggcttgc tgcttcaccc ttggggatga aataatggaa gctggttaaa gtggatgagg | 6301 |
| ttggaaagag ttgccataat gaggtcccac gtggcttctt cgataggagc cacaacttgg | 6361 |
| ggtgggaaga acttgtccct caggcttgtt gccctctgca gttgatctcc aaagttttaa | 6421 |
| acctgttaaa ttaattttga caaataagtt accctcaact cagatcaaaa atgggcagcc | 6481 |
| aagtcttcgg taggaattgg agccggtgta attcctccct aagaggcaac ctgttgaatt | 6541 |
| tactctctca gagtaaatgg tgggaaggga tccctttgta tactttttta aatactacaa | 6601 |
| attagtgtca ggcagttccc agaaagagac aagaaatcct agtggcctcc cagactgcag | 6661 |
| ggtccccaag gatggaaagg gaatgttctg ctggttctac cctgtttgtt gtgtcttgct | 6721 |
| atacagaaaa accacatttc ttttatatac tgtacgtggg catatcttgt tgttcagttt | 6781 |
| gggtgtctgc taaagaggaa gtgcactggc cctctttgaa agggctttac agtgggggca | 6841 |
| ccaagacccc aaagggccca ggccaggaga ctgttaaagt gaaaaggcaa tctatgactc | 6901 |
| accttgctct gccatccctg gcagccccca ccggtgtcct gttcctgcca catggagctt | 6961 |
| gacttcatgc cagctataat ctcccctgcc ttcctttaat cccaatttcc cctgctcact | 7021 |
| cttccacaga tataaagaac aaacacttag catcccacac tcaccccttc taatcctgaa | 7081 |
| gggaagccca ttctaaactc ctttcctgca aacccatttc cagctcctag tagctttcct | 7141 |
| cccaaaggct ttctttccaa tcctttatag ctttggagac gcctccccaa ttccccaggg | 7201 |
| aaggaaactg ttgtgtccaa tccccattaa agacaaattg atcagtgctt cccactccaa | 7261 |
| gtcaagcttt atgcaggaat gcttttccat cagggaataa atacttagaa gcgcttacaa | 7321 |
| ggtgccaggc acctcctttc tgcatgtgcc tgcctttcta gtagcagaca gatggaaaca | 7381 |
| ttgtctcatt ttgtcaagga gtccaaagaa atgattataa aaccaggatt catccttctt | 7441 |
| ctccagaaag attttttttt aagtaaacac ctttcaatcc ccaacacaag ctgcttcaca | 7501 |
| actccaggct agaaggcagg agagcgatct gatgtgtttc tttcatttgc cagaattcct | 7561 |
| gataccaaaa gcctctctct ctgttgagta acctctcaag gaccagagtg gagtccagat | 7621 |
| tgttaggctc agatcaaggg tggggaaata ctgccctctc gtggtggctt ttcatccagg | 7681 |
| cctcgtagcc aaccgtttaa gtgcaaaata gaattaagca atgggtaagc aaaatagggt | 7741 |
| tgacaagata tttgggggtt attcgggtta tggcccattt atttccctct tccccctgaa | 7801 |
| ttgaccagta gcagctccag ccccatttca caaaagtgag tttggccagg aggaatgaga | 7861 |
| cgtctcctga aataggaaca ccggaacatc atgctcacct gccatcacta tgcatccagt | 7921 |
| tcccacagct tgtgtcgtga aagagcagag agatgatgtt aaactccttg ggaggagaga | 7981 |
| gggcttcttt tggtttccct ggagtgagac agccaggtgt ctttcttttg cggggggaca | 8041 |
| cttcagaccc atcaatatgg aattttggga gccgacctga gtgcaaatcc taattctgcc | 8101 |
| cctgttggtg cagatggctg tgggcggctc acttgacctt ttagagtctg catacccacc | 8161 |
| tgtataacaa ggtggattga atgagacaat gcccacgaaa tgcccagtta cagtacctgg | 8221 |
| ttcaaaactt actgcatttt aatttttcac ttaacttata acatgtcttg cttctccagt | 8281 |
| gtgtggaagg caccgggcag tttgcagaga taagcaaaac acagttcctc tcgtgcagaa | 8341 |
| ggttagaatc tatttttttt tttgacagag tcttgctctg tcacccaggc tggcgtacag | 8401 |
| tggtacgatc tcagctcact gcatcctctg cctcccccag ttcaagtgat tcttctgcct | 8461 |
| cggcctcctg agtaactggg actacaggcg cctaccacca cgcccagcta agttttgtat | 8521 |
| ttttagtaga gtcagggttt caccatgttg gccaggctgg tcttgaattc ctgacctcaa | 8581 |
| atgatccacg cacctcagcc tcccaaagtg ctggattaca ggcatgagcc accacgccca | 8641 |
| gccaaaggtt ataatctgat ggagagagac acccgtcttg gaactgacat aaatttctgg | 8701 |
| ggtttgagaa atgggcggga tttcactggt agcttctgga aggtaagagt tgtccaggaa | 8761 |
| ttgggaagag tgagaggaaa ggcacggaca gggagcatgt aagataaatt gaggctggct | 8821 |
| ttggaaggct gaggagggtg agaaaaggtg ggctgggacc agaccgtggg gagaggtgag | 8881 |
| tggcattaca agaaatttag gctttattca gaaggcaaca gggagtccct aagaatgttt | 8941 |
| ttcaaaaagg gacattaagg cgattggagt tatacttgga aaagaaagtt ctggccacag | 9001 |
| tacagagcat ggcccgttga gctgttgggg gggttattgc tgcaaccaag gcttgagtga | 9061 |
| gggaagaggc ggatgtagtg ataaagagac tccaggaact gaatcagcgt acctggcacc | 9121 |
| ccatccattg tagagggtga gaataaagga gaaattaaag catcttgcag gctgggcgcg | 9181 |
| gtagctcatg tctgtaatcc cagcactttg ggaggccgag gtgggtgtat cagttgaggt | 9241 |
| caggagttgg agaccagtca gccagttagt agaaaccctg actctactaa gaaaatacaa | 9301 |
| aaattagctg ggcatggtgg catgcgcctg tagtctcagc tacctgggag gctgaggaag | 9361 |
| gaggatcgct tgagcccagg aggtggaggc tgcagtgagc caagattgta ccactgcact | 9421 |
| ccagcctggg tgacagagca agactcttat ctcaaaaaaa ataaaataaa ataaaataaa | 9481 |
| ataaaacatc ttgcccctag ctgagagaga ggtctctgaa gagcaggctc agggaaaaga | 9541 |
| tgagttttca gagctgatgt gatagtcagc ttctctggag tcaacagggt gaatccttcc | 9601 |
| caagtccagc catgcccaga tgcccggagg gaaaactgac ccccagccag tagacattgg | 9661 |
| ctaagaacac agaatcttct gaccaaacac gctttcagca gctgcctgct ctggactttg | 9721 |
| aaagaggtca ggtcttgccc taagctcaaa acaagtgaga ggtgtcctga cctagctcat | 9781 |
| agggcaaatg gtcctaatag gatgggcaat ccagatgcct gagccccttc actccgacag | 9841 |
| caccagcgcc taatgcagcc ttttcattct tgccattagg aaatctgtgg acttctagcc | 9901 |
| tgtgttttaa accagccatg tttccttgta tatttcccta cccgctgccc cacataccca | 9961 |
| gcatgccgct gtggccacca tgtcctcaaa gccttctgtc tgtatcagga atgtagtctg | 10021 |
| agactgccag gaagcaacaa ggagagagaa acactaacta gtcttccttt ataacccatt | 10081 |
| catactctct ggctgtcccc aaccttcata gtctcctgca tccaaatgtc ctctttggct | 10141 |
| caaaaagtag gccaggcatg gtggttcatg cctgtaatag cactttggga gactgaggtg | 10201 |
| ggaggatcac ttggggccag gagtttgaga ccagcttggg caacacagcg caatctcgtc | 10261 |
| tctactaaaa aaaaaaaaaa aaaaaaatta gctgggcatg atggcatgct cctgtggtcc | 10321 |
| cagctacttg ggaggctgag gcaggaggat cacttggtcc caggagtttg aggcgacagt | 10381 |
| gagctaggat cgcaccactg cactccagcc tgagtgacag agcaagaccc tgtctctaaa | 10441 |
| aaaaattaaa atgaaagacc aggtgctggg attaaggaaa cacaggtctg agggtctgag | 10501 |
| ggaaggggcc tgcctcccag ggagtcaaca tagatgttcc ccatgaacag ggatttgact | 10561 |
| ttggaggcca acctggcctg gcctctgccc tttatctcac actccctatc cttggcccac | 10621 |
| tgccagtccc tgccttgtgg caaaggggcc ccaaaagaaa agctgccctt ccccaaatgt | 10681 |
| aaggacccag gtacactttc acccgtggaa agcagtgtct gtcgagagtc tgtttcctat | 10741 |
| taatacttat caaagccatg tgcgagggag gtggtcagct gtcaatatgc cttagtatgt | 10801 |
| ttatatgagt ttgttttgtt ctaaaatacc caaacagttc tggtcaagcg gggctatgcc | 10861 |
| cgtctggccc aaaacacagt ccgttattaa cgagatggcc ctggcaggcg ggaacaaatc | 10921 |
| tgcctccatg cactgcttcc tgtagtcttt tagaaagtaa ctccaggaca tcgaagtgcc | 10981 |
| cagatttgac tcctaagttc taggagactg tagcgcaggg tctgtcaacc ttagcactat | 11041 |
| tggcatttgg ggctgggtaa ttctttcttg tgggggccgt cttgggtact gtaggaagct | 11101 |
| gagcagcatt cctggcctcc atccacaaga tacctgtagc agtgtcctgc caacggtaac | 11161 |
| aatcaagtat gtcatcagac attgcccaat gtccccaggg ggcaacaccc ctctcttgga | 11221 |
| cttcagggtc aagagaatct ctgctggcta ccccaggact tctcattata gatttcctgg | 11281 |
| agcacgcagc agaaactttg cctagcccag tggttgtttc cattatctgc tgccaaagtg | 11341 |
| ggatttgagg gtgtccgggg gagggggcat ggggagggca gtatgctttc aaaaacccct | 11401 |
| cccaggccag gcgtggtggc tcatgcctgt aatcacagga ctttgggagg ccgaggctgg | 11461 |
| cagatcactt gaggctggga gttagagacc aacctggcta acatggcaaa acctcgtctc | 11521 |
| tactaaaaat acaaaaatca gcccggcgtg gtggcgggca tctgtaatcc catctactcg | 11581 |
| ggaggctgag gcaggagaat tacttgaacc caggaggcag aggctgcagt gagccgagat | 11641 |
| ggcaccactg cactccagct tgttgacaga atgagaccct gtggaaaaaa aaaaaaaagc | 11701 |
| cctcccatgc cagaacagag gatggcagtc tgtttcaata agacactgtg tccttggtgt | 11761 |
| tggttctgat taagactcac tgagatccag tgctcttgag ctgggtctca gtcccctccc | 11821 |
| atgtcctgtg ctctgccgcc actgttttca ttgttgtgtt ctcgttgtga ttgttaagac | 11881 |
| tcacactcct ggctcagcag tggttttcca gaaggcccaa agagcggtgc cgggcacccc | 11941 |
| acgtcgcagt gtccgttccg ggcttgggaa gctggggagg tgggcagacc tggtcgcatc | 12001 |
| tcaccacaca cacacacaca cacacacaca cacacgctgt cagaaactcg gccgtccccc | 12061 |
| ctacctctga gctctcaatg ctgctaatct ctgccaagtg tccctgtgct ccagcacctt | 12121 |
| ccttgaagga ctgacgccca ccccacgctc tttgcgaggt tgtccaggct gtgtttgtcg | 12181 |
| catgctcttc ttctgtatag ttctcatctt ccaattttat gggattcaac aaaagcctat | 12241 |
| tatgcttgtt tgcattatgg ttacaatatt aaaaagtgga ttcaaaaaaa a | |

As used herein, specific residues of nNOSμ can be referred to as, for example, “nNOSμ(30),” which refers to the 30^{th} amino acid residue relative to SEQ ID NO: 1. Specific domains of nNOSμ can be referred to by such nomenclature as well. The N-terminal or “scaffolding domain” of nNOSμ can be referred to as nNOSμ(1-305) of SEQ ID NO: 1 or SEQ ID NO: 3, for example. The “enzymatic domain” or “catalytic domain” can be referred to as nNOSμ(306-1468) of SEQ ID NO: 1 or SEQ ID NO: 4. Similarly, the “μ insert,” which distinguishes nNOSμ from the other nNOS isoforms can be referred to as nNOSμ(845-878) of SEQ ID NO: 1 or SEQ ID NO: 5, as described herein.

As used herein, the term “fusion polypeptide” or “fusion protein” refers to a protein created by joining two polypeptide coding sequences together. The fusion polypeptides described herein are fusion polypeptides formed by joining a coding sequence of nNOSμ or functional fragment thereof with a coding sequence of a second polypeptide comprising a plasma membrane targeting sequence to form a fusion or chimeric coding sequence such that they constitute a single open-reading frame. The fusion coding sequence, when transcribed and translated, expresses a fusion polypeptide. In other words, a “fusion polypeptide” or “fusion protein” is a recombinant protein of two or more proteins which are joined by a peptide bond.

As used herein, the term “fused” means that one protein is physically associated with a second protein, for example via an electrostatic or hydrophobic interaction or a covalent linkage. Covalent linkage can encompass linkage as a fusion protein or chemically coupled linkage, for example via a cysteine residue.

The term “functional fragment” as used in the context of a “functional fragment of nNOSμ” or “nNOSμ functional fragment thereof” refers to a fragment of nNOSμ polypeptide that improves or treats one or more symptoms or parameters of a neurodegenerative disorder such as, for example decreased maximum tetanic force, and/or increased susceptibility to fatigue when targeted to the sarcolemma via fusion with a membrane targeting sequence as described herein. Accordingly, the term “functional” when used in conjunction with a fragment, “derivative” or “variant” refers to a protein molecule that possesses a biological activity that is substantially similar to a biological activity of the entity or molecule of which it is a fragment, derivative or variant. By “substantially similar” in this context is meant that the biological activity, e.g., ability to increase skeletal muscle SpF, and/or decrease susceptibility to fatigue is at least 50% as active as a reference, e.g., a corresponding wild-type nNOSμ polypeptide targeted to the sarcolemma, and preferably at least 60% as active, 70% as active, 80% as active, 90% as active, 95% as active, 100% as active or even higher (i.e., the variant or derivative has greater activity than the wild-type), e.g., 110% as active, 120% as active, or more. Assays to measure the biological activity of an nNOSμ fusion polypeptide comprising an nNOSμ functional fragment of interest are known in the art, and non-limiting examples are provided herein in the Examples. Such assays to measure the biological activity of an nNOSμ functional fragment include, for example, in situ analysis of skeletal muscle contractile function, resistance to exercise-induced fatigue, resistance to stretch contraction-induced injury, and in vitro analysis of diaphragm muscle function. In some embodiments, an assay to measure the biological activity of an nNOSμ functional fragment can involve transfecting muscle fibers, such as flexor digitorum brevis, from an Mdx mouse model with an nNOSμ fusion protein comprising an nNOSμ functional fragment of interest, performing short-term culture of the muscle fibers, and measuring nitric oxide production using an extracellular NO-specific electrode. In some embodiments, an assay to measure the biological activity of an nNOSμ functional fragment can involve transfecting muscle fibers, such as flexor digitorum brevis, from an Mdx mouse model with an nNOSμ fusion protein comprising an nNOSμ functional fragment of interest via in vivo electroporation, and measuring nitric oxide production using an extracellular NO-specific electrode.

Accordingly, in some embodiments of the aspects described herein, the nNOSμ fusion proteins or derivatives thereof comprise a functional fragment of the nNOSμ scaffolding domain. The nNOSμ scaffolding domain has a sequence of MEDHMFGVQQIQPNVISVRLFKRKVGGLGFLVKERVSKPPVIISDLIRGGAAEQSGLIQAGDIIL AVNGRPLVDLSYDSALEVLRGIASETHVVLILRGPEGFTTHLETTFTGDGTPKTIRVTQPLGPPT KAVDLSHQPPAGKEQPLAVDGASGPGNGPQHAYDDGQEAGSLPHANGLAPRPPGQDPAKKA TRVSLQGRGENNELLKEIEPVLSLLTSGSRGVKGGAPAK AEMKDMGIQVDRDLDGKSHK PLPLGVENDR VFNDLWGKGN VPVVLNNPYS EKEQPPTSGK QSPTKNGSPSKCPRF (SEQ ID NO: 3).

In some embodiments of the aspects described herein, the nNOSμ fusion proteins or derivatives thereof comprise a functional fragment of the nNOSμ enzymatic or catalytic domain. The nNOSμ enzymatic or catalytic domain has a sequence of LKVKN WETEVVLTDT LHLKSTLETG CTEYICMGSI MHPSQHARRP EDVRTKGQLFPLAKEFIDQY YSSIKRFGSK AHMERLEEVN KEIDTTSTYQ LKDTELIYGA KHAWRNASRCVGRIQWSKLQ VFDARDCTTA HGMFNYICNH VKYATNKGNL RSAITIFPQR TDGKHDFRVWNSQLIRYAGY KQPDGSTLGD PANVQFTEIC IQQGWKPPRG RFDVLPLLLQ ANGNDPELFQIPPELVLEVP IRHPKFEWFK DLGLKWYGLP AVSNMLLEIG GLEFSACPFS GWYMGTEIGVRDYCDNSRYN ILEEVAKKMN LDMRKTSSLW KDQALVEINI AVLYSFQSDK VTIVDHHSATESFIKHMENE YRCRGGCPAD WVWIVPPMSG SITPVFHQEM LNYRLTPSFE YQPDPWNTHVWKGTNGTPTK RRAIGFKKLA EAVKFSAKLM GQAMAKRVKA TILYATETGK SQAYAKTLCEIFKHAFDAKV MSMEEYDIVH LEHETLVLVV TSTFGNGDPP ENGEKFGCAL MEMRHPNSVQEERKYPEPLR FFPRKGPPLP NGDTEVHGLA AARDSQHRSY KVRFNSVSSY SDSQKSSGDGPDLRDNFESA GPLANVRFSV FGLGSRAYPH FCAFGHAVDT LLEELGGERI LKMREGDELCGQEEAFRTWA KKVFKAACDV FCVGDDVNIE KANNSLISND RSWKRNKFRL TFVAEAPELTQGLSNVHKKR VSAARLLSRQ NLQSPKSSRS TIFVRLHTNG SQELQYQPGD HLGVFPGNHEDLVNALIERL EDAPPVNQMV KVELLEERNT ALGVISNWTD ELRLPPCTIF QAFKYYLDITTPPTPLQLQQ FASLATSEKE KQRLLVLSKG LQEYEEWKWG KNPTIVEVLE EFPSIQMPATLLLTQLSLLQ PRYYSISSSP DMYPDEVHLT VAIVSYRTRD GEGPIHHGVC SSWLNRIQADELVPCFVRGA PSFHLPRNPQ VPCILVGPGT GIAPFRSFWQ QRQFDIQHKG MNPCPMVLVFGCRQSKIDHI YREETLQAKN KGVFRELYTA YSREPDKPKK YVQDILQEQL AESVYRALKEQGGHIYVCGD VTMAADVLKA IQRIMTQQGK LSAEDAGVFI SRMRDDNRYH EDIFGVTLRTYEVTNRLRSE SIAFIEESKK DTDEVFSS (SEQ ID NO: 4).

In some embodiments of the aspects described herein, the nNOSμ fusion proteins or derivatives thereof comprise a functional fragment of the nNOSμ μ insert domain. In some embodiments of the aspects described herein, an nNOSμ functional fragment comprises all or part of the nNOSμ μ insert domain. The nNOSμ μ insert domain has a sequence of YPEPLRFFPRKGPPLP NGDTEVHGLA AARDSQHR (SEQ ID NO: 5).

In addition to the nNOSμ functional fragment, the nNOSμ fusion proteins described herein further comprise a plasma membrane targeting sequence. As used herein, a “plasma membrane targeting sequence” refers to an amino acid sequence (or, depending on context, nucleotide sequence encoding the amino acid sequence), that allows a protein, such as the nNOSμ fusion proteins described herein, to be targeted or localized to the plasma membrane of a cell, or sarcolemma of a muscle cell. Such a sequence can be added, in some embodiments, to or at the C-terminus, N-terminus, or within the sequence of the nNOSμ functional fragment provided it does not disrupt any key structural or catalytic determinants necessary to maintain the desired biological activity of the NOSμ functional fragment. The nNOSμ fusion proteins described herein can be tethered or otherwise attached to a cell membrane by various means, including utilizing known and natural sequences from membrane and/or cell surface proteins which are naturally attached to the cell membrane, or any such other means known or available in the art to attach or associate a peptide or molecule with the membrane or cell surface, including via attachment to or association with a membrane protein. Sequences from known and natural membrane and/or cell surface proteins serve to transverse or attach to the membrane per se or are recognized and function to signal attachment and/or specific modification in cells. Such sequences will be known and recognized to the skilled artisan and include for instance such sequences or domains as palmitoylation sites, myristoylation sites, transmembrane domains, and PH domains.

The nNOSμ fusion proteins described herein can be attached to the membrane surface through lipid anchors or electrostatic binding. Lipid anchors are fatty acids or isoprenoids (geranyl, farnesyl) which are covalently linked to amino acids and provide a close attachment yet lateral mobility along the membrane surface. Lipid anchors include, for example, palmitoylation, and myristoylation. Palmitoylation is acquired post-translationally and cytoplasmically and not in the ER. A common recognition site for palmitoylation is C-A-A-X (SEQ ID NO: 6), with A denoting aliphatic amino acid and X any C-terminal amino acid. Myristoylation is coupled to protein translation, with co-translational modification at the N-terminus by N-myristoyltransferase (NMT). An N-terminal glycine, followed by a either an Asn, Gln, Ser, Val or Leu (and not a Asp, D-Asn, Phe or Tyr) is recognized by the NMT.

Accordingly, in some embodiments of the aspects described herein, the plasma membrane targeting sequence of an nNOSμ fusion protein or derivative thereof comprises a palmitoylation sequence. Non-limiting examples of palmitoylation sequences for use as plasma membrane targeting sequences in the nNOSμ functional fusion proteins described herein include the k-Ras palmitoylation signal sequence of KDGKKKKKKSKTKCVIM (SEQ ID NO: 6) and C-A-A-X (SEQ ID NO: 7), where A denotes an aliphatic amino acid and X any C-terminal amino acid. In some embodiments of the aspects described herein, a palmitoylation sequence is placed at the C-terminus of an NOSμ functional fragment. In some embodiments of the aspects described herein, a palmitoylation sequence is placed at the N-terminus of an NOSμ functional fragment. In some embodiments of the aspects described herein, a palmitoylation sequence is placed within a sequence of an NOSμ functional fragment. As will be understood by one of ordinary skill in the art, the placement of a plasma membrane targeting sequence, such as a palmitoylation sequence, relative to the NOSμ functional fragment should not disrupt any key structural or catalytic determinants necessary to maintain the desired biological activity of the NOSμ functional fragment.

As used herein, the term “protein” and “polypeptide” are used interchangeably. The terms “polypeptide” and “protein” are used interchangeably to refer to a polymer of linearly arranged amino acid residues linked by peptide bonds, whether produced biologically, recombinantly, or synthetically and whether composed of naturally occurring or non-naturally occurring amino acids. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, as used herein, a “polypeptide” refers to a protein that includes modifications, such as deletions, additions, and substitutions (generally conservative in nature, as would be known to a person skilled in the art) to the native sequence, as long as the protein maintains the desired functional activity. These modifications can be deliberate, as through site-directed mutagenesis, or can be accidental, such as through mutations of hosts that produce the proteins, or errors due to PCR amplification or other recombinant DNA methods.

The term “amino acid,” as used herein, is intended to refer to any natural or unnatural amino acid, whether made naturally or synthetically, including any such in L- or D-configuration. The term can include a modified or unusual amino acid or a synthetic derivative of an amino acid, e.g. diamino butyric acid and diamino propionic acid and the like. In the context of a polypeptide, an amino acid is synonymous with amino acid residue, as understood by one of ordinary skill in the art.

The terms “analog” or “variant” as used herein in reference to nNOSμ fusion proteins refers to a polypeptide or nucleic acid that differs from the naturally occurring polypeptide, i.e., nNOSμ or a domain thereof, or nucleic acid encoding such polypeptide, by one or more amino acid or nucleic acid deletions, additions, substitutions or side-chain modifications, yet retains one or more functions or biological activities (i.e., functions or activities specific to muscle function) of the naturally occurring molecule, i.e., nNOSμ. Amino acid substitutions include alterations in which an amino acid is replaced with a different naturally-occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Substitutions encompassed by variants as described herein may also be “non conservative,” in which an amino acid residue which is present in a peptide is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). A “variant” of nNOSμ polypeptide or functional fragement thereof refers to a molecule substantially similar in structure and function to that of a polypeptide of SEQ ID NO: 1, where the function is, for example, the ability to ability to increase skeletal muscle mass, increase maximum tetanic force, and/or decrease susceptibility to fatigue. For example, the term “analog” encompasses an nNOSμ fusion polypeptide comprising one or more conservative amino acid changes relative to the sequence of wild-type nNOSμ. Polypeptide analogs can be prepared by, for example, peptide synthesis. Any combination of deletion, insertion, and substitution is made to arrive at the final polypeptide fusion protein variant provided that the final variant possesses the desired characteristics, e.g., can target the sarcolemma and can improve or restore muscle function.

Amino acid sequence insertions include intrasequence insertions of single or multiple amino acid residues. Another type of variant is an amino acid substitution analog. These variants have at least one amino acid residue in the portion of the nNOSμ fusion protein comprising the wild-type nNOSμ replaced by a different residue. In some embodiments, the substitution peptide variant comprises one or more conservative substitutions. In some embodiments, the substitution peptide variant comprises one or more non-conservative substitutions. In some embodiments, the substitution peptide variant comprises one or more non-conservative substitutions and one or more conservative substitutions.

Accordingly, in some embodiments, an nNOSμ fusion protein variant comprises an nNOSμ functional fragment that differs by 1 conservative substitution, 2 conservative substitutions, 3 conservative substitutions, 4 conservative substitutions, 5 conservative substitutions, 6 conservative substitutions, 7 conservative substitutions, 8 conservative substitutions, 9 conservative substitutions, 10 or fewer conservative substitutions, 15 or fewer conservative substitutions, 20 or fewer conservative substitutions, 25 or fewer conservative substitutions, 305 or fewer conservative substitutions, 35 or fewer conservative substitutions, 40 or fewer conservative substitutions, 45 or fewer conservative substitutions, or 50 or fewer conservative substitutions, relative to the sequence of the nNOSμ naturally occurring molecule or a domain or portion thereof having the desired biological activity.

In some embodiments, an nNOSμ fusion protein variant comprises an nNOSμ functional fragment that differs by 1 or fewer non-conservative substitutions, 2 or fewer non-conservative substitutions, 3 or fewer non-conservative substitutions, 4 or fewer non-conservative substitutions, 5 or fewer non-conservative substitutions, 6 or fewer non-conservative substitutions, 7 or fewer or non-conservative substitutions, 8 or fewer non-conservative substitutions, 9 or fewer non-conservative substitutions, or 10 or fewer or non-conservative substitutions, relative to the sequence of the nNOSμ naturally occurring molecule or a domain or portion thereof having the desired biological activity.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, an nNOSμ fusion protein is an nNOSμ fusion protein derivative. As used herein the term “derivative” refers to a polypeptide that is derived from an wild-type nNOSμ as described herein, e.g., a fragment or processed form of nNOSμ, and includes peptides which have been chemically modified by techniques such as adding additional side chains, ubiquitination, labeling, pegylation (derivatization with polyethylene glycol), and insertion, deletion or substitution of amino acid mimetics and/or unnatural amino acids that do not normally occur in the sequence of wild-type nNOSμ that is basis of the derivative. For example, in some embodiments, the nNOSμ fusion protein derivatives can comprise a label, such as, for example, an epitope, e.g., a FLAG epitope or a V5 epitope or an HA epitope. Such a tag can be useful for, for example, purifying the fusion protein derivative. The term “derivative” also encompasses a derivatized polypeptide, such as, for example, a polypeptide modified to contain one or more-chemical moieties other than an amino acid. The chemical moiety can be linked covalently to the peptide, e.g., via an amino terminal amino acid residue, a carboxy terminal amino acid residue, or at an internal amino acid residue. Such modifications include the addition of a protective or capping group on a reactive moiety in the polypeptide, addition of a detectable label, and other changes that do not adversely destroy the activity of the nNOSμ fusion protein. In some embodiments, an nNOSμ derivative contains additional chemical moieties not normally a part of the molecule. Such moieties can improve its solubility, absorption, biological half life, etc. The moieties can alternatively decrease the toxicity of the molecule, or eliminate or attenuate an undesirable side effect of the molecule, etc. Moieties capable of mediating such effects are disclosed, for example, in Remington's Pharmaceutical Sciences, 18th edition, A. R. Gennaro, Ed., MackPubl., Easton, Pa. (1990).

The nNOSμ fusion protein derivatives described herein can also comprise unnatural amino acids or modifications of N or C terminal amino acids. Examples of such unnatural or modified amino acids include, but are not limited to, acedisubstituted amino acids, N-alkyl amino acids, lactic acid, 4-hydroxy proline, γ-carboxyglutamate, ε-N,N,N-trimethyllysine, ornithine, ε-N-acetyllysine, O-phosphoserine, N-acetylserine, N-formylmethionine, 3-methylhistidine, 5-hydroxylysine, δ-N-methylarginine. Other modified amino acids homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-aminophenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, desamino-Tyr, aminovaleric acid, pyroglutaminic acid, alpha-aminoisobutyric acid, gamma-aminobutyric acid, alpha-aminobutyric acid, alpha,gamma-aminobutyric acid□□pyridylalanine, α-napthyalanine, β-napthyalanine, Ac-β-napthyalanine, N^{ε}-picoloyl-lysine, 4-halo-Phenyl, 4-pyrolidylalanine, isonipecotic carboxylic acid, and any combinations thereof.

Methods of Delivering and Administering nNOSμ Fusion Proteins

Expression of the nNOSμ fusion proteins in a cell can result from delivery of the fusion protein to the cell or by delivery of a polynucleotide, such as a DNA or RNA, encoding the fusion protein to a cell, wherein the polynucleotide is transcribed, and the transcript is translated, to generate the nNOSμ fusion protein. Trans-splicing, polypeptide cleavage and polypeptide ligation can also be involved in expression of an nNOSμ protein in a cell.

Accordingly, in some aspects, also provided herein are gene therapy vectors and methods thereof for the in vivo production of the nNOSμ fusion proteins described herein. Such therapies achieve therapeutic effects by introduction of the polynucleotide sequences into cells or tissues in a subject having any of the neuromuscular disorders described herein.

As used herein, the term “vector” refers to a nucleic acid molecule capable of transporting or mediating expression of a heterologous nucleic acid to which it has been linked, i.e., an nNOSμ fusion protein, to a host cell; a plasmid is a species of the genus encompassed by the term “vector.” The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome. Preferred vectors are those capable of autonomous replication and/or expression of nucleic acids to which they are linked.

In some embodiments of the aspects described herein, delivery of polynucleotide sequences encoding the nNOSμ fusion proteins described herein can be achieved using a recombinant expression vector. Various viral vectors which can be utilized for gene therapy include, for example, adenovirus, herpes virus, vaccinia, or an RNA virus such as a retrovirus. The retroviral vector can be a derivative of a murine or avian retrovirus. Such expression methods have been used in gene delivery and are well known in the art. For example, in regard to muscle-specific gene delivery, US patent application No. 20110212529 describes muscle-specific expression vectors comprising muscle-specific enhancers and promoter elements derived from a muscle creatine kinase promoter and enhancers, a troponin I promoter and internal regulatory elements, a skeletal alpha-actin promoter, or a desmin promoter and enhancers; and “Gene therapy of mdx mice with large truncated dystrophins generated by recombination using rAAV6,” Odom G L, Gregorevic P, Allen J M, Chamberlain J S. Mol Ther. 2011 January; 19(1):36-45; “rAAV6-microdystrophin rescues aberrant Golgi complex organization in mdx skeletal muscles,” Percival J M, Gregorevic P, Odom G L, Banks G B, Chamberlain J S, Froehner S C. Traffic. 2007 October; 8(10):1424-39; and “rAAV6-microdystrophin preserves muscle function and extends lifespan in severely dystrophic mice,” Gregorevic P, Allen J M, Minami E, Blankinship M J, Haraguchi M, Meuse L, Finn E, Adams M E, Froehner S C, Murry C E, Chamberlain J S. Nat Med. 2006 July; 12(7):787-9, describe, in part, muscle-specific gene therapy methods. In addition, U.S. patent application No. 2002/0,193,335 provides methods of delivering a gene therapy vector, or transformed cell, to neurological tissue; U.S. patent application No. 2002/0,187,951 provides methods for treating or preventing a neurodegenerative disease in a mammal by administering a lentiviral vector to a target cell in the brain or nervous system of the mammal; U.S. patent application No. 2002/0,107,213 discloses a gene therapy vehicle and methods for its use in the treatment and prevention of neurodegenerative disease; U.S. patent application No. 2003/0,099,671 discloses a mutated rabies virus suitable for delivering a gene to a subject; and U.S. Pat. No. 6,310,196 describes a DNA construct which is useful for immunization or gene therapy; U.S. Pat. No. 6,436,708 discloses a gene delivery system which results in long-term expression throughout the brain; and U.S. Pat. No. 6,140,111 which disclose retroviral vectors suitable for human gene therapy in the treatment of a variety of diseases.

Retroviruses provide a convenient platform for gene delivery. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09. Examples of retroviral vectors in which a heterologous gene can be inserted include, but are not limited to: Moloney murine leukemia virus (MoMuLV), Harvey murine sarcoma virus (HaMuSV), murine mammary tumor virus (MuMTV), and Rous sarcoma virus (RSV). A number of additional retroviral vectors can incorporate multiple genes. All of these vectors can transfer or incorporate a gene for a selectable marker so that transduced cells can be identified and generated. By inserting, for example, a polynucleotide sequence encoding an nNOSμ fusion protein of interest into the viral vector, along with another gene which encodes a ligand for a receptor on a specific target cell, such as, for example, a muscle cell, the vector is now target specific.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

In some embodiments of the aspects described herein, a nucleotide sequence encoding an nNOSμ fusion protein is inserted into an adenovirus-based expression vector. Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76).

The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the ITRs exactly at the genome ends.

Adenoviral vectors have several advantages in gene therapy. They infect a wide variety of cells, have a broad host-range, exhibit high efficiencies of infectivity, direct expression of heterologous genes at high levels, and achieve long-term expression of those genes in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. For all these reasons vectors derived from human adenoviruses, in which at least the E1 region has been deleted and replaced by a gene of interest, have been used extensively for gene therapy experiments in the preclinical and clinical phase.

Adenoviral vectors for use with the present invention can be derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, U.S. Pat. No. 6,048,551, incorporated herein by reference in its entirety, describes replication-deficient adenoviral vectors that can be used to include an nNOSμ fusion proteinunder the control of the Rous Sarcoma Virus (RSV) promoter.

Other recombinant adenoviruses of various serotypes, and comprising different promoter systems, can be created by those skilled in the art. See, e.g., U.S. Pat. No. 6,306,652, incorporated herein by reference in its entirety.

Moreover, “minimal” adenovirus vectors as described in U.S. Pat. No. 6,306,652 will find use with the present invention. Such vectors retain at least a portion of the viral genome required for encapsidation (the encapsidation signal), as well as at least one copy of at least a functional part or a derivative of the ITR. Packaging of the minimal adenovirus vector can be achieved by co-infection with a helper virus or, alternatively, with a packaging-deficient replicating helper system.

Other useful adenovirus-based vectors for delivery of an nNOSμ fusion protein include the “gutless” (helper-dependent) adenovirus in which the vast majority of the viral genome has been removed. Wu et al. (2001) Anesthes. 94:1119-32. Such “gutless” adenoviral vectors produce essentially no viral proteins, thus allowing gene therapy to persist for over a year after a single administration. Parks (2000) Clin. Genet. 58:1-11; Tsai et al. (2000) Curr. Opin. Mol. Ther. 2:515-23. In addition, removal of the viral genome creates space that can be used to insert control sequences that provide for regulation of transgene expression by systemically administered drugs (Burcin et al. (1999) Proc. Natl. Acad. Sci. USA 96:355-60), adding both safety and control of virally driven protein expression. These and other recombinant adenoviruses will find use with the present methods.

Another viral system that can be used, in some embodiments, for gene delivery of the nNOSμ fusion proteins described herein is AAV. AAV is a parvovirus which belongs to the genus Dependovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation.

The AAV genome is a linear single-stranded DNA molecule containing approximately 4681 nucleotides. The AAV genome generally comprises an internal non-repeating genome flanked on each end by inverted terminal repeats (ITRs). The ITRs are approximately 145 base pairs (bp) in length. The ITRs have multiple functions, including serving as origins of DNA replication and as packaging signals for the viral genome.

The internal non-repeated portion of the genome includes two large open reading frames, known as the AAV replication (rep) and capsid (cap) genes. The rep and cap genes code for viral proteins that allow the virus to replicate and package the viral genome into a virion. In particular, a family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

Adeno-associated virus (AAV) has been used with success in gene therapy. AAV has been engineered to deliver genes of interest by deleting the internal nonrepeating portion of the AAV genome (i.e., the rep and cap genes) and inserting a heterologous gene (in this case, the gene encoding the anti-inflammatory cytokine) between the ITRs. The heterologous gene is typically functionally linked to a heterologous promoter (constitutive, cell-specific, or inducible) capable of driving gene expression in the patient's target cells under appropriate conditions.

Accordingly, recombinant AAV virions comprising an nNOSμ fusion protein nucleic acid or peptide/protein can be produced using a variety of art-recognized techniques. In some embodiments, a rAAV vector construct is packaged into rAAV virions in cells co-transfected with wild-type AAV and a helper virus, such as adenovirus. See, e.g., U.S. Pat. No. 5,139,941.

Alternatively, plasmids can be used to supply the necessary replicative functions from AAV and/or a helper virus. In some embodiments, rAAV virions are produced using a plasmid to supply necessary AAV replicative functions (the “AAV helper functions”). See e.g., U.S. Pat. Nos. 5,622,856 and 5,139,941, both incorporated herein by reference in their entireties. In another embodiment, a triple transfection method is used to produce rAAV virions. The triple transfection method is described in detail in U.S. Pat. Nos. 6,001,650 and 6,004,797, which are incorporated by reference herein in their entireties. The triple transduction method is advantageous because it does not require the use of an infectious helper virus during rAAV production, enabling production of a stock of rAAV virions essentially free of contaminating helper virus. This is accomplished by use of three vectors for rAAV virion production: an AAV helper function vector, an accessory function vector, and a rAAV expression vector. One of skill in the art will appreciate, however, that the nucleic acid sequences encoded by these vectors can be provided on two or more vectors in various combinations. Vectors and cell lines necessary for preparing helper virus-free rAAV stocks are commercially available as the AAV Helper-Free System (Catalog No. 240071) (Stratagene/Agilent, La Jolla, Calif.).

The AAV helper function vector encodes AAV helper function sequences (i.e., rep and cap) that function in trans for productive rAAV replication and encapsidation. Preferably, the AAV helper function vector supports efficient rAAV virion production without generating any detectable replication competent AAV virions (i.e., AAV virions containing functional rep and cap genes). An example of such a vector, pHLP19, is described in U.S. Pat. No. 6,001,650. The rep and cap genes of the AAV helper function vector can be derived from any of the known AAV serotypes. For example, the AAV helper function vector may have a rep gene derived from AAV-2 and a cap gene derived from AAV-6. One of skill in the art will recognize that other rep and cap gene combinations are possible, the defining feature being the ability to support rAAV virion production.

The accessory function vector encodes nucleotide sequences for non-AAV-derived viral and/or cellular functions upon which AAV is dependent for replication (the “accessory functions”). The accessory functions include those functions required for AAV replication, including, without limitation, genes involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In one embodiment, the accessory function plasmid pLadeno5 can be used. See U.S. Pat. No. 6,004,797. This plasmid provides a complete set of adenovirus accessory functions for AAV vector production, but lacks the components necessary to form replication-competent adenovirus.

Unlike stocks of rAAV vectors prepared using infectious helper virus, stocks prepared using an accessory function vector (e.g. the triple transfection method) do not contain contaminating helper virus because no helper virus is added during rAAV production. Even after purification, for example by CsCl density gradient centrifugation, rAAV stocks prepared using helper virus still remain contaminated with some level of residual helper virus. When adenovirus is used as the helper virus in preparing a stock of rAAV virions, contaminating adenovirus can be inactivated by heating to temperatures of approximately 60° C. for 20 minutes or more. This treatment effectively inactivates only the helper virus since AAV is extremely heat stable, while the helper adenovirus is heat labile. Although heat inactivating of rAAV stocks may render much of the contaminating adenovirus non-infectious, it does not physically remove the helper virus proteins from the stock. Such contaminating viral protein can elicit undesired immune responses in subjects and are to be avoided if possible. Contaminating adenovirus particles and proteins in rAAV stocks can be avoided by use of the accessory function vectors disclosed herein.

Recombinant AAV expression vectors can be constructed using standard techniques of molecular biology. rAAV vectors comprise a transgene of interest flanked by AAV ITRs at both ends. rAAV vectors are also constructed to contain transcription control elements operably linked to the transgene sequence, including a transcriptional initiation region and a transcriptional termination region. The control elements are selected to be functional in a mammalian target cell.

The nucleotide sequences of AAV ITR regions are known. See, e.g., Kotin (1994) Human Gene Therapy 5:793-801; Berns “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAV-6, AAV-7 and AAV-8, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable transgenes for delivery in AAV vectors can be less than about 5 kilobases (kb) in size. The selected polynucleotide sequence is operably linked to control elements that direct the transcription thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, neuron-specific enolase promoter, a GFAP promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

The AAV expression vector harboring a transgene of interest, i.e., a sequence encoding an nNOSμ polypeptide, bounded by AAV ITRs can be constructed by directly inserting the selected sequence(s) into an AAV genome that has had the major AAV open reading frames (“ORFs”) excised. Other portions of the AAV genome can also be deleted, so long as enough of the ITRs remain to provide replication and packaging functions. Such constructs can be designed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-96; Vincent et al. (1990) Vaccines 90 (Cold Spring Harbor Laboratory Press); Carter (1992) Current Opinion in Biotechnology 3:533-39; Muzyczka (1992) Current Topics in Microbiol. and Immunol 158:97-129; Kotin (1994) Human Gene Therapy 5:793-801; Shelling and Smith (1994) Gene Therapy 1:165-69; and Zhou et al. (1994) J. Exp. Med. 179:1867-75.

AAV ITR-containing DNA fragments can be ligated at both ends of a selected transgene using standard techniques, such as those described in Sambrook et al., supra. For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

Suitable host cells for producing rAAV virions of the present invention from rAAV expression vectors include microorganisms, yeast cells, insect cells, and mammalian cells. Such host cells are preferably capable of growth in suspension culture, a bioreactor, or the like. The term “host cell” includes the progeny of the original cell that has been transfected with an rAAV virion. Cells from the stable human cell line, 293 (readily available through the American Type Culture Collection under Accession Number ATCC CRL1573) are preferred in the practice of the present invention. The human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

Additional viral vectors useful for delivering the nucleic acid molecules and/or expressing an nNOSμ fusion protein, in some embodiments, include those derived from the pox family of viruses, including vaccinia virus and avian poxvirus. By way of example, vaccinia virus recombinants expressing a an nNOSμ fusion proteincan be constructed as follows. DNA carrying the nNOSμ fusion protein is inserted into an appropriate vector adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter and the gene into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can be used to express an nNOSμ fusion protein, in some embodiments. Recombinant avipox viruses expressing immunogens from mammalian pathogens are known to confer protective immunity when administered to non-avian species. The use of avipox vectors in human and other mammalian species is advantageous with regard to safety because members of the avipox genus can only productively replicate in susceptible avian species. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors, can also be used for gene delivery, in some embodiments. Michael et al. (1993) J. Biol. Chem. 268:6866-69 and Wagner et al. (1992) Proc. Natl. Acad. Sci. USA 89:6099-6103. Members of the Alphavirus genus, for example the Sindbis and Semliki Forest viruses, may also be used as viral vectors for delivering and expressing an nNOSμ fusion protein. See, e.g., Dubensky et al. (1996) J. Virol. 70:508-19; WO 95/07995; WO 96/17072.

In some embodiments of the aspects described herein, another targeted delivery system for a polynucleotide encoding an nNOSμ fusion protein is a colloidal dispersion system. Colloidal dispersion systems include, for example, macromolecule complexes, nanocapsules, microspheres, beads, and lipid-based systems including oil-in-water emulsions, micelles, mixed micelles, and liposomes. Liposomes are artificial membrane vesicles which are useful as delivery vehicles in vitro and in vivo. RNA, DNA and intact virions can be encapsulated within the aqueous interior and be delivered to cells in a biologically active form (see, for example, Fraley, et al. (1981) Trends Biochem. Sci., 6: 77). Methods for efficient gene transfer using a liposome vehicle, are known in the art (see, for example, Mannino, et al. (1988) Biotechniques, 6: 682. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine and distearoylphosphatidylcholine. The targeting of liposomes is also possible based on, for example, organ-specificity, cell-specificity, and organelle-specificity and is known in the art.

There is a wide range of methods which can be used to deliver the cells expressing nNOSμ fusion protein to a site for use in treating a neuromuscular disorder. In some embodiments, cells expressing an nNOSμ fusion protein can be delivered by direct application, for example, direct injection of a sample of such cells into a target site, such as muscle tissue. These cells can be purified. In some embodiments, such cells can be delivered in a medium or matrix which partially impedes their mobility so as to localize the cells to a target site. Such a medium or matrix could be semi-solid, such as a paste or gel, including a gel-like polymer. Alternatively, in some embodiments, the medium or matrix could be in the form of a solid, a porous solid which will allow the migration of cells into the solid matrix, and hold them there while allowing proliferation of the cells.

In some embodiments, the delivery vehicle is an adeno-associated viral vector (AAV) or a recombinant adeno-associated AAV (rAAV). In some such embodiments, the AAV vector or rAAV vector includes an expression cassette comprising a polynucleotide sequence encoding the nNOSμ fusion protein.

In some embodiments of the aspects described herein, the therapeutic compositions can also include a delivery vehicle to facilitate the delivery of nNOSμ fusion protein to target muscle cells. In some embodiments, the delivery vehicle is a cell-penetrating peptide. Cell-penetrating peptides (CPPs, also known as protein transduction domains, membrane translocating sequences, and Trojan peptides) are short peptides (less than or equal to approximately 40 amino acids), which are able to penetrate a cell membrane to gain access to the interior of a cell. Thus, CPPs can be used to facilitate the transfer of proteins to a muscle cell in vivo. Cell penetrating peptides have been used in exon skipping to deliver oligonucleotides to the muscle cell (Wu et al. 2008; Ivanova et al. 2008; Jearawiriyapaisarn et al.; Betts et al. 2012; Moulton 2012; Yin et al. 2008; Yin et al. 2010). In addition, the TAT PTD, when attached to recombinant full-length utrophin and micro-utrophin protein, has been able to successfully transfer utrophin proteins to the muscle of mdx mice (Sonnemann et al. 2009).

CPPs that can be used in accordance with the embodiments described herein include, but are not limited to, Penetratin or Antenapedia PTD (RQIKWFQNRRMKWKK; SEQ ID NO:8), TAT (YGRKKRRQRRR; SEQ ID NO:9) or a modified TAT having one or more mutated residues (e.g., YARAAARQARA; SEQ ID NO:10), R9-Tat GRRRRRRRRRPPQ; SEQ ID NO:11), R10 (RRRRRRRRRR; SEQ ID NO:12) SynB1 (RGGRLSYSRRRFSTSTGR; SEQ ID NO:13), SynB3 (RRLSYSRRRF; SEQ ID NO:14), PTD-4 (PIRRRKKLRRLK; SEQ ID NO:15), PTD-5 (RRQRRTSKLMKR SEQ ID NO:16), FHV Coat-(35-49) (RRRRNRTRRNRRRVR; SEQ ID NO:17), BMV Gag-(7-25) (KMTRAQRRAAARRNRWTAR; SEQ ID NO:18), HTLV-II Rex-(4-16) (TRRQRTRRARRNR; SEQ ID NO:19), D-Tat (GRKKRRQRRRPPQ; SEQ ID NO:20), Transportan chimera (GWTLNSAGYLLGKINLKALAALAKKIL; SEQ ID NO:21), MAP (KLALKLALKLALALKLA; SEQ ID NO:22), SBP (MGLGLHLLVLAAALQGAWSQPKKKRKV; SEQ ID NO:23), FBP (GALFLGWLGAAGSTMGAWSQPKKKRKV; SEQ ID NO:24), MPG (ac-GALFLG FLGAAGSTMGAWSQPKKKRKV-cya; SEQ ID NO:25), MPG.sup.(.DELTA.NLS) (ac-GALFLGFLGAAGSTMGAWSQPKSKRKV-cya; SEQ ID NO:26). Pep-1 (ac-KETWWETWWTEWSQPKKKRKV-cya; SEQ ID NO:27), Pep-2 (ac-KETWFETWFTEWSQPKKKRKV-cya; SEQ ID NO:28), or any other suitable CPP known to one of ordinary skill in the art.

To facilitate the efficient delivery of an nNOSμ fusion protein to a muscle cell, a cell penetrating peptide, such as the TAT protein transduction domain (PTD), can be attached to or conjugated to an nNOSμ fusion protein via a covalent linkage (e.g., an intra-molecular form of chemical bonding that is characterized by the sharing or one or more pairs of electrons between two components, producing a mutual attraction that holds the resultant molecule together) or a non-covalent linkage (e.g., an interaction—not covalent in nature—that provides force to hold the molecules or parts of molecules together, such as ionic bonds, hydrophobic interactions, hydrogen bonds, van-der-Waals forces, and dipole-dipole bonds), in accordance with methods known in the art. Where the attachment or conjugation involves a covalent linkage, the CPP and the nNOSμ fusion protein or functional fragment thereof can be directly coupled to each other or can be coupled via a linker molecule. In some embodiments, a covalent linkage can be between nucleotide molecules. In such embodiments, a nucleotide sequence that encodes the CPP can be operably linked to a polynucleotide sequence encoding an nNOSμ fusion protein, so that when expressed by a vector (e.g., a plasmid or a viral vector), the CPP-nNOSμ fusion protein is expressed as a single fusion protein.

In some embodiments of the aspects described herein, an nNOSμ fusion protein is delivered to a cell or administered to a subject in the form of a modified RNA encoding the nNOSμ fusion protein, wherein the modified RNA molecule comprises one or more modifications, such that introducing the modified RNA molecules to a cell results in a reduced innate immune response relative to a cell contacted with synthetic RNA molecules encoding the nNOSμ fusion protein not comprising the one or more modifications.

Modified-RNAs encoding the nNOSμ fusion proteins described herein include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein. Specific examples of modified-RNA compositions useful with the methods described herein include, but are not limited to, RNA molecules containing modified or non-natural internucleoside linkages. Modified-RNAs having modified internucleoside linkages include, among others, those that do not have a phosphorus atom in the internucleoside linkage. In other embodiments, the modified-RNA has a phosphorus atom in its internucleoside linkage(s).

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference in its entirety.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of modified oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, each of which is herein incorporated by reference in its entirety.

Some embodiments of the modified-RNAs encoding nNOSμ fusion proteins described herein include nucleic acids with phosphorothioate internucleoside linkages and oligonucleosides with heteroatom internucleoside linkage, and in particular —CH2-NH—CH2-, —CH2-N(CH3)-O—CH2- [known as a methylene (methylimino) or MMI], —CH2-O—N(CH3)-CH2-, —CH2-N(CH3)-N(CH3)-CH2- and —N(CH3)-CH2-CH2- [wherein the native phosphodiester internucleoside linkage is represented as —O—P—O—CH2-] of the above-referenced U.S. Pat. No. 5,489,677, and the amide backbones of the above-referenced U.S. Pat. No. 5,602,240, both of which are herein incorporated by reference in their entirety. In some embodiments, the nucleic acid sequences featured herein have morpholino backbone structures of the above-referenced U.S. Pat. No. 5,034,506, herein incorporated by reference in its entirety.

Modified-RNAs encoding nNOSμ fusion proteins described herein can also contain one or more substituted sugar moieties. The nucleic acids featured herein can include one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10 alkyl or C2 to C10 alkenyl and alkynyl. Exemplary modifications include O[(CH2)nO] mCH3, O(CH2).nOCH3, O(CH2)nNH2, O(CH2) nCH3, O(CH2)nONH2, and O(CH2)nON[(CH2)nCH3)]2, where n and m are from 1 to about 10. In some embodiments, synthetic, modified-RNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, a reporter group, an intercalator, a group for improving the pharmacokinetic properties of an RNA, or a group for improving the pharmacodynamic properties of a synthetic, modified-RNA, and other substituents having similar properties. In some embodiments, the modification includes a 2′ methoxyethoxy (2′-O—CH2CH2OCH3, also known as 2′-O-(2-methoxyethyl) or 2′-MOE) (Martin et al., Helv. Chim Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, and 2′-dimethylaminoethoxyethoxy (also known in the art as 2′-O-dimethylaminoethoxyethyl or 2′-DMAEOE), i.e., 2′-O—CH2-O—CH2-N(CH2)2.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid sequence, particularly the 3′ position of the sugar on the 3′ terminal nucleotide or in 2′-5′ linked nucleotides and the 5′ position of 5′ terminal nucleotide. A synthetic, modified-RNA can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

As non-limiting examples, modified-RNAs encoding nNOSμ fusion proteins described herein can include at least one modified nucleoside including a 2′-O-methyl modified nucleoside, a nucleoside comprising a 5′ phosphorothioate group, a 2′-amino-modified nucleoside, 2′-alkyl-modified nucleoside, morpholino nucleoside, a phosphoramidate or a non-natural base comprising nucleoside, or any combination thereof.

In some embodiments of this aspect and all other such aspects described herein, the at least one modified nucleoside is selected from the group consisting of 5-methylcytidine (5mC), N6-methyladenosine (m6A), 3,2′-O-dimethyluridine (m4U), 2-thiouridine (s2U), 2′ fluorouridine, pseudouridine, 2′-O-methyluridine (Um), 2′ deoxyuridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m6₂Am), 2′-O-methylcytidine (Cm), 7-methylguanosine (m7G), 2′-O-methylguanosine (Gm), N2,7-dimethylguanosine (m2,7G), N2, N2, 7-trimethylguanosine (m2,2,7G), and inosine (I).

Alternatively, a modified-RNA encoding an nNOSμ fusion protein can comprise at least two modified nucleosides, at least 3, at least 4, at least 5, at least 6, at least 7, at least 8, at least 9, at least 10, at least 15, at least 20 or more, up to the entire length of the oligonucleotide. It is not necessary for all positions in a given modified-RNA to be uniformly modified. However, it is preferred, but not absolutely necessary, that each occurrence of a given nucleoside in a molecule is modified (e.g., each cytosine is a modified cytosine e.g., 5mC). However, it is also contemplated that different occurrences of the same nucleoside can be modified in a different way in a given modified-RNA molecule (e.g., some cytosines modified as 5mC, others modified as 2′-O-methylcytidine or other cytosine analog). The modifications need not be the same for each of a plurality of modified nucleosides in a modified-RNA. Furthermore, in some embodiments of the aspects described herein, a modified-RNA comprises at least two different modified nucleosides. In some such preferred embodiments of the aspects described herein, the at least two different modified nucleosides are 5-methylcytidine and pseudouridine. A synthetic, modified-RNA can also contain a mixture of both modified and unmodified nucleosides.

As used herein, “unmodified” or “natural” nucleosides or nucleobases include the purine bases adenine (A) and guanine (G), and the pyrimidine bases thymine (T), cytosine (C) and uracil (U). In some embodiments, a synthetic, modified-RNA comprises at least one nucleoside (“base”) modification or substitution. Modified nucleosides include other synthetic and natural nucleobases such as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, O6-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleosides also include natural bases that comprise conjugated moieties, e.g. a ligand. As discussed herein above, the RNA containing the modified nucleosides must be translatable in a host cell (i.e., does not prevent translation of the polypeptide encoded by the modified RNA). For example, transcripts containing s2U and m6A are translated poorly in rabbit reticulocyte lysates, while pseudouridine, m5U, and m5C are compatible with efficient translation. In addition, it is known in the art that 2′-fluoro-modified bases useful for increasing nuclease resistance of a transcript, leads to very inefficient translation. Translation can be assayed by one of ordinary skill in the art using e.g., a rabbit reticulocyte lysate translation assay.

Further modified nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in Int. Appl. No. PCT/US09/038425, filed Mar. 26, 2009; those disclosed in The Concise Encyclopedia Of Polymer Science And Engineering, pages 858-859, Kroschwitz, J. L, ed. John Wiley & Sons, 1990, and those disclosed by Englisch et al., Angewandte Chemie, International Edition, 1991, 30, 613.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety, and U.S. Pat. No. 5,750,692, also herein incorporated by reference in its entirety.

Another modification for use with the synthetic, modified-RNAs described herein involves chemically linking to the RNA one or more ligands, moieties or conjugates that enhance the activity, cellular distribution or cellular uptake of the RNA. Ligands can be particularly useful where, for example, a synthetic, modified-RNA is administered in vivo. Such moieties include but are not limited to lipid moieties such as a cholesterol moiety (Letsinger et al., Proc. Natl. Acid. Sci. USA, 1989, 86: 6553-6556, herein incorporated by reference in its entirety), cholic acid (Manoharan et al., Biorg. Med. Chem. Let., 1994, 4:1053-1060, herein incorporated by reference in its entirety), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770, each of which is herein incorporated by reference in its entirety), a thiocholesterol (Oberhauser et al., Nucl. Acids Res., 1992, 20:533-538, herein incorporated by reference in its entirety), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54, each of which is herein incorporated by reference in its entirety), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783, each of which is herein incorporated by reference in its entirety), a polyamine or a polyethylene glycol chain (Manoharan et al., Nucleosides & Nucleotides, 1995, 14:969-973, herein incorporated by reference in its entirety), or adamantane acetic acid (Manoharan et al., Tetrahedron Lett., 1995, 36:3651-3654, herein incorporated by reference in its entirety), a palmityl moiety (Mishra et al., Biochim Biophys. Acta, 1995, 1264:229-237, herein incorporated by reference in its entirety), or an octadecylamine or hexylamino-carbonyloxycholesterol moiety (Crooke et al., J. Pharmacol. Exp. Ther., 1996, 277:923-937, herein incorporated by reference in its entirety).

The modified-RNAs encoding nNOSμ fusion proteins described herein can further comprise a 5′ cap. In some embodiments of the aspects described herein, the synthetic, modified-RNAs comprise a 5′ cap comprising a modified guanine nucleotide that is linked to the 5′ end of an RNA molecule using a 5′-5′triphosphate linkage. As used herein, the term “5′ cap” is also intended to encompass other 5′ cap analogs including, e.g., 5′ diguanosine cap, tetraphosphate cap analogs having a methylene-bis(phosphonate) moiety (see e.g., Rydzik, A M et al., (2009) Org Biomol Chem 7(22):4763-76), dinucleotide cap analogs having a phosphorothioate modification (see e.g., Kowalska, J. et al., (2008) RNA 14(6):1119-1131), cap analogs having a sulfur substitution for a non-bridging oxygen (see e.g., Grudzien-Nogalska, E. et al., (2007) RNA 13(10): 1745-1755), N7-benzylated dinucleoside tetraphosphate analogs (see e.g., Grudzien, E. et al., (2004) RNA 10(9):1479-1487), or anti-reverse cap analogs (see e.g., Jemielity, J. et al., (2003) RNA 9(9): 1108-1122 and Stepinski, J. et al., (2001) RNA 7(10):1486-1495). In one such embodiment, the 5′ cap analog is a 5′ diguanosine cap. In some embodiments, the synthetic, modified RNA does not comprise a 5′ triphosphate.

The 5′ cap is important for recognition and attachment of an mRNA to a ribosome to initiate translation. The 5′ cap also protects the synthetic, modified-RNA from 5′ exonuclease mediated degradation. It is not an absolute requirement that a synthetic, modified-RNA comprise a 5′ cap, and thus in other embodiments the synthetic, modified-RNAs lack a 5′ cap. However, due to the longer half-life of synthetic, modified-RNAs comprising a 5′ cap and the increased efficiency of translation, synthetic, modified-RNAs comprising a 5′ cap are preferred herein.

The modified-RNAs encoding nNOSμ fusion proteins described herein can further comprise a 5′ and/or 3′ untranslated region (UTR). Untranslated regions are regions of the RNA before the start codon (5′) and after the stop codon (3′), and are therefore not translated by the translation machinery. Modification of an RNA molecule with one or more untranslated regions can improve the stability of an mRNA, since the untranslated regions can interfere with ribonucleases and other proteins involved in RNA degradation. In addition, modification of an RNA with a 5′ and/or 3′ untranslated region can enhance translational efficiency by binding proteins that alter ribosome binding to an mRNA. Modification of an RNA with a 3′ UTR can be used to maintain a cytoplasmic localization of the RNA, permitting translation to occur in the cytoplasm of the cell. In one embodiment, the synthetic, modified-RNAs described herein do not comprise a 5′ or 3′ UTR. In another embodiment, the synthetic, modified-RNAs comprise either a 5′ or 3′ UTR. In another embodiment, the synthetic, modified-RNAs described herein comprise both a 5′ and a 3′ UTR. In one embodiment, the 5′ and/or 3′ UTR is selected from an mRNA known to have high stability in the cell (e.g., a murine alpha-globin 3′ UTR). In some embodiments, the 5′ UTR, the 3′ UTR, or both comprise one or more modified nucleosides.

In some embodiments, the modified-RNAs encoding nNOSμ fusion proteins described herein further comprise a Kozak sequence. The “Kozak sequence” refers to a sequence on eukaryotic mRNA having the consensus (gcc)gccRccAUGG (SEQ ID NO: 29), where R is a purine (adenine or guanine) three bases upstream of the start codon (AUG), which is followed by another ‘G’. The Kozak consensus sequence is recognized by the ribosome to initiate translation of a polypeptide. Typically, initiation occurs at the first AUG codon encountered by the translation machinery that is proximal to the 5′ end of the transcript. However, in some cases, this AUG codon can be bypassed in a process called leaky scanning. The presence of a Kozak sequence near the AUG codon will strengthen that codon as the initiating site of translation, such that translation of the correct polypeptide occurs. Furthermore, addition of a Kozak sequence to a synthetic, modified-RNA will promote more efficient translation, even if there is no ambiguity regarding the start codon. Thus, in some embodiments, the modified-RNAs described herein further comprise a Kozak consensus sequence at the desired site for initiation of translation to produce the correct length polypeptide. In some such embodiments, the Kozak sequence comprises one or more modified nucleosides.

In some embodiments, the modified-RNAs encoding nNOSμ fusion proteins described herein further comprise a “poly (A) tail”, which refers to a 3′ homopolymeric tail of adenine nucleotides, which can vary in length (e.g., at least 5 adenine nucleotides) and can be up to several hundred adenine nucleotides). The inclusion of a 3′ poly(A) tail can protect the synthetic, modified-RNA from degradation in the cell, and also facilitates extra-nuclear localization to enhance translation efficiency. In some embodiments, the poly(A) tail comprises between 1 and 500 adenine nucleotides; in other embodiments the poly(A) tail comprises at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 225, at least 250, at least 275, at least 300, at least 325, at least 350, at least 375, at least 400, at least 425, at least 450, at least 475, at least 500 adenine nucleotides or more. In one embodiment, the poly(A) tail comprises between 1 and 150 adenine nucleotides. In another embodiment, the poly(A) tail comprises between 90 and 120 adenine nucleotides. In some such embodiments, the poly(A) tail comprises one or more modified nucleosides.

A modified-RNA encoding an nNOSμ fusion protein can be introduced into a cell in any manner that achieves intracellular delivery of the modified-RNA, such that expression of the polypeptide encoded by the modified RNA can occur. As used herein, the term “transfecting a cell” refers to the process of introducing nucleic acids into cells using means for facilitating or effecting uptake or absorption into the cell, as is understood by those skilled in the art. As the term is used herein, “transfection” does not encompass viral- or viral particle based delivery methods. Absorption or uptake of a synthetic, modified RNA can occur through unaided diffusive or active cellular processes, or by auxiliary agents or devices. Further approaches are described herein below or known in the art.

A modified-RNA encoding an nNOSμ fusion protein can be introduced into a target cell, for example, by transfection, nucleofection, lipofection, electroporation (see, e.g., Wong and Neumann, Biochem. Biophys. Res. Commun. 107:584-87 (1982)), microinjection (e.g., by direct injection of a modified RNA), biolistics, cell fusion, and the like. In an alternative embodiment, a modified RNA can be delivered using a drug delivery system such as a nanoparticle, a dendrimer, a polymer, a liposome, or a cationic delivery system. Positively charged cationic delivery systems facilitate binding of a modified RNA (negatively charged polynucleotides) and also enhances interactions at the negatively charged cell membrane to permit efficient cellular uptake. Cationic lipids, dendrimers, or polymers can either be bound to modified RNAs, or induced to form a vesicle or micelle (see e.g., Kim S H., et al (2008) Journal of Controlled Release 129(2):107-116) that encases the modified RNA. Methods for making and using cationic-modified RNA complexes are well within the abilities of those skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205, which are incorporated herein by reference in their entirety).

In some embodiments of the aspects described herein, the composition further comprises a reagent that facilitates uptake of a modified-RNA encoding an nNOSμ fusion protein into a cell (transfection reagent), such as an emulsion, a liposome, a cationic lipid, a non-cationic lipid, an anionic lipid, a charged lipid, a penetration enhancer or alternatively, a modification to the synthetic, modified RNA to attach e.g., a ligand, peptide, lipophillic group, or targeting moiety.

The process for delivery of a modified-RNA encoding an nNOSμ fusion protein to a cell will necessarily depend upon the specific approach for transfection chosen. One preferred approach is to add the RNA, complexed with a cationic transfection reagent directly to the cell culture media for the cells.

In certain embodiments of the aspects described herein, a synthetic, modified RNA can be introduced into target cells by transfection or lipofection. Suitable agents for transfection or lipofection include, for example, calcium phosphate, DEAE dextran, lipofectin, lipofectamine, DIMRIE C, SUPERFECT, and EFFECTIN (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

A modified-RNA encoding an nNOSμ fusion protein can be transfected into target cells as a complex with cationic lipid carriers (e.g., Oligofectamine) or non-cationic lipid-based carriers (e.g., Transit-TKOTM, Mirus Bio LLC, Madison, Wis.). Successful introduction of a modified RNA into host cells can be monitored using various known methods. For example, transient transfection can be signaled with a reporter, such as a fluorescent marker, such as Green Fluorescent Protein (GFP). Successful transfection of a modified RNA can also be determined by measuring the protein expression level of the target polypeptide by e.g., Western Blotting or immunocytochemistry.

In some embodiments of the aspects described herein, the modified-RNA encoding an nNOSμ fusion protein is introduced into a cell using a transfection reagent. Some exemplary transfection reagents include, for example, cationic lipids, such as lipofectin (Junichi et al, U.S. Pat. No. 5,705,188), cationic glycerol derivatives, and polycationic molecules, such as polylysine (Lollo et al., PCT Application WO 97/30731). Examples of commercially available transfection reagents include, for example LIPOFECTAMINE™ (INVITROGEN; Carlsbad, Calif.), LIPOFECTAMINE 2000™ (INVITROGEN; Carlsbad, Calif.), 293FECTIN™ (INVITROGEN; Carlsbad, Calif.), CELLFECTIN™ (INVITROGEN; Carlsbad, Calif.), DMRIE-C™ (INVITROGEN; Carlsbad, Calif.), FREESTYLE™ MAX (INVITROGEN; Carlsbad, Calif.), LIPOFECTAMINE™ 2000 CD (INVITROGEN; Carlsbad, Calif.), LIPOFECTAMINE™ (INVITROGEN; Carlsbad, Calif.), RNAIMAX (INVITROGEN; Carlsbad, Calif.), OLIGOFECTAMINE™ (INVITROGEN; Carlsbad, Calif.), OPTIFECT™ (INVITROGEN; Carlsbad, Calif.), X-TREMEGENE Q2 TRANSFECTION REAGENT (ROCHE; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), TRANSFECTAM® Reagent (PROMEGA; Madison, Wis.), TRANSFAST™ Transfection Reagent (PROMEGA; Madison, Wis.), TFX™-20 Reagent (PROMEGA; Madison, Wis.), TFX™-50 Reagent (Promega; Madison, Wis.), DREAMFECT™ (OZ Biosciences; Marseille, France), ECOTRANSFECT (OZ Biosciences; Marseille, France), TRANSPASS^{a} D1 Transfection Reagent (New England Biolabs; Ipswich, Mass., USA), LYOVEC™/LIPOGEN™ (INVITROGEN; San Diego, Calif., USA), PERFECTIN Transfection Reagent (GENLANTIS; San Diego, Calif., USA), NEUROPORTER Transfection Reagent (GENLANTIS; San Diego, Calif., USA), GENEPORTER Transfection reagent (GENLANTIS; San Diego, Calif., USA), GENEPORTER 2 Transfection reagent (GENLANTIS; San Diego, Calif., USA), CYTOFECTIN Transfection Reagent (GENLANTIS; San Diego, Calif., USA), BACULOPORTER Transfection Reagent (GENLANTIS; San Diego, Calif., USA), TROGANPORTER™ transfection Reagent (GENLANTIS; San Diego, Calif., USA), RIBOFECT (BIOLINE; Taunton, Mass., USA), PLASFECT (BIOLINE; Taunton, Mass., USA), UNIFECTOR (B-Bridge International; Mountain View, Calif., USA), SUREFECTOR (B-Bridge International; Mountain View, Calif., USA), or HIFECT™ (B-Bridge International, Mountain View, Calif., USA), among others.

In other embodiments, highly branched organic compounds, termed “dendrimers,” can be used to bind the exogenous nucleic acid and introduce it into the cell.

In other embodiments of the aspects described herein, non-chemical methods of transfection are contemplated. Such methods include, but are not limited to, electroporation (methods whereby an instrument is used to create micro-sized holes transiently in the plasma membrane of cells under an electric discharge), sono-poration (transfection via the application of sonic forces to cells), and optical transfection (methods whereby a tiny (˜1 μm diameter) hole is transiently generated in the plasma membrane of a cell using a highly focused laser). In other embodiments, particle-based methods of transfections are contemplated, such as the use of a gene gun, whereby the nucleic acid is coupled to a nanoparticle of an inert solid (commonly gold) which is then “shot” directly into the target cell's nucleus; “magnetofection,” which refers to a transfection method, that uses magnetic force to deliver exogenous nucleic acids coupled to magnetic nanoparticles into target cells; “impalefection,” which is carried out by impaling cells by elongated nanostructures, such as carbon nanofibers or silicon nanowires which have been coupled to exogenous nucleic acids.

Other agents may be utilized to enhance the penetration of the administered nucleic acids, including glycols, such as ethylene glycol and propylene glycol, pyrrols such as 2-pyrrol, azones, and terpenes, such as limonene and menthone.

In some embodiments of the aspects described herein, particularly embodiments involving in vivo administration of modified-RNA encoding an nNOSμ fusion protein or compositions thereof, the modified-RNAs described herein are formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

The compositions described herein can be formulated into any of many possible administration forms, including a sustained release form. The compositions can be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

The compositions described herein can be prepared and formulated as emulsions for the delivery of modified-RNA encoding an nNOSμ fusion protein. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain further components in addition to the dispersed phases, and the active drug (i.e., synthetic, modified-RNA) which can be present as a solution in either the aqueous phase, oily phase or itself as a separate phase. Pharmaceutical excipients such as emulsifiers, stabilizers, dyes, and anti-oxidants can also be present in emulsions as needed. Emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

As noted above, liposomes can optionally be prepared to contain surface groups to facilitate delivery of liposomes and their contents to specific cell populations. For example, a liposome can comprise a surface groups such as antibodies or antibody fragments, small effector molecules for interacting with cell-surface receptors, antigens, and other like compounds.

Surface groups can be incorporated into the liposome by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

A number of liposomes comprising nucleic acids are known in the art. WO 96/40062 (Thierry et al.) discloses methods for encapsulating high molecular weight nucleic acids in liposomes. U.S. Pat. No. 5,264,221 (Tagawa et al.) discloses protein-bonded liposomes and asserts that the contents of such liposomes can include an RNA molecule. U.S. Pat. No. 5,665,710 (Rahman et al.) describes certain methods of encapsulating oligodeoxynucleotides in liposomes. WO 97/04787 (Love et al.) discloses liposomes comprising RNAi molecules targeted to the raf gene. In addition, methods for preparing a liposome composition comprising a nucleic acid can be found in e.g., U.S. Pat. Nos. 6,011,020; 6,074,667; 6,110,490; 6,147,204; 6,271,206; 6,312,956; 6,465,188; 6,506,564; 6,750,016; and 7,112,337. Each of these approaches can provide delivery of a synthetic, modified-RNA as described herein to a cell.

In some embodiments of the aspects described herein, the modified-RNA encoding an nNOSμ fusion protein described herein can be encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al, Electrolyte- and pH-stabilities of aqueous solid lipid nanoparticle (SLN) dispersions in artificial gastrointestinal media. Eur J Pharm Biopharm 52:203. 2001), the contents of which are herein incoporated in their entireties by reference.

Other compositions and methods of preparing and delivering modified RNAs for use with the nNOSμ fusion proteins described herein can be found, for example, in WO2013151736, WO 2014028429, WO 2013151665, WO 2012019168, U.S. Pat. No. 8,710,200, WO 2013151670, US 20130259924, WO 2013151664, US 20130156849, US 20130245104, US 20140147454, and US 20120237975, the contents of each of which are herein incorporated by reference in their entireties.

For administration to a subject in need thereof, e.g., a subject diagnosed with or predisposed to a neuromuscular disorder, the nNOSμ fusion proteint and/or viral expression vector and/or modified RNA encoding the nNOSμ fusion protein can be provided in a pharmaceutically acceptable composition. As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutically acceptable composition can further comprise one or more pharmaceutically carriers (additives) and/or diluents. As used herein, the term “pharmaceutically-acceptable carrier” means a pharmaceutically-acceptable material, composition or vehicle, such as a liquid, diluent, excipient, manufacturing aid or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include, but are not limited to, gelatin, buffering agents, such as magnesium hydroxide and aluminum hydroxide, pyrogen-free water, isotonic saline, Ringer's solution, pH buffered solutions, bulking agents such as polypeptides and amino acids, serum component such as serum albumin, HDL and LDL, and other non-toxic compatible substances employed in pharmaceutical formulations. Preservatives and antioxidants can also be present in the formulation. The terms such as “excipient”, “carrier”, “pharmaceutically acceptable carrier” or the like are used interchangeably herein.

Pharmaceutically acceptable carriers can vary in a composition of the invention, depending on the administration route and formulation. For example, the pharmaceutically acceptable composition of the invention can be delivered via injection. These routes for administration (delivery) include, but are not limited to, subcutaneous or parenteral including intravenous, intracortical, intracranial, intramuscular, intraperitoneal, and infusion techniques. In another embodiment, the pharmaceutical composition is formulated for intramuscular injection.

The nNOSμ fusion protein and/or the composition thereof can be formulated in pharmaceutically acceptable compositions which comprise a therapeutically-effective amount of the compound, formulated together with one or more pharmaceutically acceptable carriers (additives) and/or diluents. The compounds can be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) sublingually; (5) ocularly; (6) transdermally; (7) transmucosally; or (8) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

When administering a pharmaceutical composition of the invention parenterally, it will be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), and suitable mixtures thereof. In some embodiments, the pharmaceutical carrier can be a buffered solution (e.g. PBS).

In some embodiments, the pharmaceutical composition can be formulated in an emulsion or a gel. In such embodiments, at least one nNOSμ fusion protein or viral vector encoding a nNOSμ fusion protein or modified RNA encoding an nNOSμ fusion protein can be encapsulated within a biocompatible gel, e.g., hydrogel and a peptide gel. The gel pharmaceutical composition can be implanted to the brain near the degenerating neuronal cells, e.g., the cells in proximity to the amyloid plaque.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to compositions of the invention, however, any vehicle, diluent, or additive used should have to be biocompatible or inert with the nNOSμ fusion protein or a vector encoding the nNOSμ fusion protein or modified RNA encoding the nNOSμ fusion protein.

The compositions can be isotonic, i.e., they can have the same osmotic pressure as blood and lacrimal fluid. The desired isotonicity of the compositions of the invention can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

In some embodiment, muscle cells transduced with a vector encoding an nNOSμ fusion protein can be included in the compositions and stored frozen. In such embodiments, an additive or preservative known for freezing cells can be included in the compositions. A suitable concentration of the preservative can vary from 0.02% to 2% based on the total weight although there may be appreciable variation depending upon the preservative or additive selected. One example of such additive or preservative can be dimethyl sulfoxide (DMSO) or any other cell-freezing agent known to a skilled artisan. In such embodiments, the composition will be thawed before use or administration to a subject, e.g., muscle stem cell therapy.

Typically, any additives (in addition to the active nNOSμ fusion protein) can be present in an amount of 0.001 to 50 wt % solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject in need thereof, and for any particular method of administration, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

The compositions of the invention can be prepared by mixing the ingredients following generally-accepted procedures. For example, an effective amount of an nNOSμ fusion protein or vectors encoding an nNOSμ fusion protein can be re-suspended in an appropriate pharmaceutically acceptable carrier and the mixture can be adjusted to the final concentration and viscosity by the addition of water or thickening agent and possibly a buffer to control pH or an additional solute to control tonicity. An effective amount of an nNOSμ fusion protein described herein and any other additional agent, e.g., for inhibiting a neuromuscular disorder, can be mixed with the cell mixture. Generally the pH can vary from about 3 to about 7.5. In some embodiments, the pH of the composition can be about 6.5 to about 7.5. Compositions can be administered in dosages and by techniques well known to those skilled in the medical and veterinary arts taking into consideration such factors as the age, sex, weight, and condition of the particular patient, and the composition form used for administration (e.g., liquid). Dosages for humans or other mammals can be determined without undue experimentation by a skilled artisan.

Suitable regimes for initial administration and further doses or for sequential administrations can be varied. In one embodiment, a therapeutic regimen includes an initial administration followed by subsequent administrations, if necessary. In some embodiments, multiple administrations of an nNOSμ fusion protein can be injected into the subject. For example, an nNOSμ fusion protein can be administered in two or more, three or more, four or more, five or more, or six or more injections. In some embodiments, the same nNOSμ fusion protein can be administered in each subsequent administration. In some embodiments, a different nNOSμ fusion protein described herein can be administered in each subsequent administration.

The subsequent injection can be administered immediately after the previous injection, or after at least about 1 minute, after at least about 2 minute, at least about 5 minutes, at least about 15 minutes, at least about 30 minutes, at least about 1 hour, at least about 2 hours, at least about 3 hours, at least about 6 hours, at least about 12 hours, at least about 24 hours, at least about 2 days, at least about 3 days, at least about 4 days, at least about 5 days, at least about 6 days or at least about 7 days. In some embodiments, the subsequent injection can be administered after at least about 1 week, at least about 2 weeks, at least about 1 month, at least about 2 years, at least about 3 years, at least about 6 years, or at least about 10 years.

In various embodiments, a dosage comprising a composition described herein is considered to be pharmaceutically effective if the dosage reduces the degree of the neuromuscular disorder, e.g., indicated by changes in muscular morphologies or improvement in muscle function, by at least about 5%, at least about 10%, at least about 15%, at least about 20%, at least about 30%, at least about 40%, or at least about 50%. In some embodiments, the muscular function is improved by more than 50%, e.g., at least about 60%, or at least about 70%. In some embodiments, the muscle function is improved by at least about 80%, at least about 90% or greater, as compared to a control (e.g. in the absence of the composition described herein).

Cells to which the vectors or modified RNAs encoding nNOSμ fusion proteins are delivered or administered include, for example, muscle cells, myoblasts, muscle progenitor cells, and stem cells, including pluripotent and multipotent stem cells.

### Neuromuscular Disorders

In some aspects, the present disclosure is directed to methods of treating neuromuscular diseases. In certain further embodiments, the neuromuscular diseases include Duchenne muscular dystrophy, Becker muscular dystrophy, Limb-girdle muscular dystrophies, Ullrich congenital muscular dystrophy, inflammatory myositis, muscle atrophy, and Amyotrophic lateral sclerosis.

As demonstrated herein, nNOSμ fusion proteins are useful in pharmaceutical compositions for and methods of treatment of neuromuscular disorders, such as muscular dystrophies. Accordingly, provided herein are methods of treating a subject having or at risk for a neuromuscular disorder comprising administering a therapeutically effective amount of an nNOSμ fusion protein or derivative thereof. In some embodiments of these methods and all such methods described herein, the nNOSμ fusion protein or derivative thereof is administered to the subject in the form of a vector or nucleic acid encoding the nNOSμ fusion protein or derivative thereof.

Neuromuscular disorders or diseases refer to those disorders in which muscle function in impaired, either directly due to pathologies of the muscle (myopathic disorders), and/or indirectly, due to pathologies of nerves or neuromuscular junctions (neuropathic disorders), and include muscular dystrophies, inflammatory myopathies, and muscle atrophies. Accordingly, as used herein the term “neuromuscular disorders” encompasses muscular dystrophies (including but not limited to severe or benign K-linked muscular dystrophy, limb-girdle dystrophy, facioscapulohumeral dystrophy, myotonic dystrophy, distal muscular dystrophy, progressive dystrophic ophthalmoplegia, oculopharyngeal dystrophy, Duchenne's muscular dystrophy, and Fakuyama-type congenital muscular dystophy); polymyositis; amyotrophic lateral sclerosis (ALS); muscle atrophy; organ atrophy; frailty; carpal tunnel syndrome; congestive obstructive pulmonary disease; congenital myopathy; myotonia congenital; familial periodic paralysis; paroxysmal myoglobinuria; myasthenia gravis; Eaton-Lambert syndrome; secondary myasthenia; denervation atrophy; paroxymal muscle atrophy; cerebrovascular accidents (stroke), Parkinson's disease, multiple sclerosis, Huntington's disease (Huntington's chorea) and CreutzfeldtJakob disease; and sarcopenia, cachexia and other muscle wasting syndromes

The term “degenerative muscle condition” refers to conditions, disorders, diseases and injuries characterized by one or more of muscle loss, muscle degeneration or wasting, muscle weakness, and defects or deficiencies in proteins associated with normal muscle function, growth or maintenance. In some embodiments, a degenerative muscle condition is sarcopenia or cachexia. In some embodiments, a degenerative muscle condition is one or more of muscular dystrophy, muscle injury, including acute muscle injury, resulting in loss of muscle tissue, muscle atrophy, wasting or degeneration, muscle overuse, muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis.

“Muscular dystrophy” refers to a group of more than 30 hereditary muscle diseases characterized by progressive skeletal muscle weakness, degeneration of skeletal muscle fibers, defects in certain muscle proteins, and death of muscle cells and tissue, which are distinguished clinically by the selective distribution of skeletal muscle weakness. Muscular dystrophies are caused by progressive degeneration of skeletal muscle fibers. Lack of one of several proteins located either at the plasma membrane or within internal membranes, increases the probability of damage during contraction, and eventually leads to fiber degeneration, accompanied by severe local inflammation with infiltration of immune-competent cells. As muscular dystrophy progresses and muscles weaken, fixations (contractures) can develop in joints, in which muscles and tendons shorten, restricting the flexibility and mobility of joints and muscles. Muscular dystrophies are multi-system disorders with manifestations in numerous body systems including the heart, gastrointestinal and nervous systems, endocrine glands, skin, eyes, and other organs. Muscular dystrophies are associated with various clinical symptoms, including muscle damage, muscle wasting, muscle weakness, muscle degeneration, muscle atrophy, weight loss, and elevated serum creatine kinase levels.

The two most common forms of muscle dystrophy are Duchenne and Becker dystrophies, each resulting from the inheritance of a mutation in the dystrophin gene, which is located at the Xp21 locus. Other muscular dystrophies include, but are not limited to, limb-girdle muscular dystrophy, fascioscapulohumeral (Landouzy-Dejerine) muscular dystrophy, congenital muscular dystrophy, myotonic dystrophy, and Emery-Dreifuss muscular dystrophy. Accordingly, as used herein, the term “muscular dystrophy” includes Duchenne Muscular Dystrophy; Becker Muscular Dystrophy; Emery-Dreifuss Muscular Dystrophy; Limb-Girdle Muscular Dystrophy; Facioscapulohumeral Muscular Dystrophy (also known as Landouzy-Dejerine); Myotonic Dystrophy; Oculopharyngeal Muscular Dystrophy; Distal Muscular Dystrophy; and Congenital Muscular Dystrophy.

Duchenne muscular dystrophy and Becker muscular dystrophy are similar in that these dystrophies share similar patterns of muscle weakness and disability and are inherited in the same way. Typically, subjects in need of treatment for Duchenne or Becker muscular dystrophies have trouble walking and eventually become wheelchair dependent. Generally, an arbitrary means of distinguishing between Duchenne muscular dystrophy and Becker muscular dystrophy depends on whether the affected subject can still walk at 16 years of age. Subjects with Duchenne muscular dystrophy are generally wheelchair bound by their teenage years. More specifically, a muscle biopsy of a subject affected with Duchenne muscular dystrophy will show more disabling change as compared to a subject affected with Becker muscular dystrophy.

Duchenne muscular dystrophy and Becker muscular dystrophy are due to defects or mutations of the same gene, which is directed to enabling muscle fibers to make dystrophin. The dystrophin gene encodes a large 427 kDa protein that functions in linking the extracellular matrix to the muscle fiber cytoskeleton. The amino terminus on dystrophin binds to filamentous actin in contact with the contractile apparatus of skeletal muscle, while a cysteine-rich domain near the carboxyl terminus binds to dystroglycan proteins localized to the fiber membrane in connection with other membrane proteins that constitute the dystrophin glycoprotein complex (DGC). The absence of dystrophin expression causes a concomitant decrease in DGC members. It is believed that loss of dystropin and the resulting DGC complex compromises the integrity of skeletal muscle membranes, which undergo damage after repeated cycles of contractile activity. Membrane damage is further thought to cause creatine kinase release, stimulate the influx of calcium, and induce the recruitment of immune T cells, macrophages, and mast cells, culminating in muscle fiber necrosis. The regenerative capacity of these cells become exhausted in Duchenne muscular dystrophy patients, thus giving way to accumulated fibrosis and fatty deposits that exacerbates the muscle wasting process.

Limb Girdle muscular dystrophies include at least ten different inherited disorders that can further be classified into two categories, autosomal-dominant (LGMD 1) and autosomal-recessive (LGMD 2) syndromes. The symptoms of most Limb Girdle muscular dystrophies typically begin with pelvic muscle weakness starting in childhood to young adulthood. Later, there is an onset of shoulder weakness with progression to significant loss of mobility or wheelchair dependence over the next 20-30 years.

The defective gene causing most autosomal-dominant type Limb Girdle muscular dystrophies has not yet been discovered, but the diseases have been linked to mutations in various chromosomes. For example, LGMD 1A type dystrophy has been linked to chromosome 5. Additionally, LGMD 1B type dystrophy has been linked to chromosome 1. Other chromosomes that have been linked to the autosomal-dominant type Limb Girdle muscular dystrophies include chromosomes 3 and 7. Several of the autosomal-recessive type Limb Girdle muscular dystrophies are due to mutations in the dystrophin-associated glycoproteins (i.e., sarcoglycans).

The compositions and methods comprising nNOSμ fusion proteins can also be used to treat an inflammatory myopathy, in some embodiments. Inflammatory myopathies refer to diseases or abnormal conditions of the striated skeletal muscles. The cause of most inflammatory myopathies is unknown. Typically, inflammatory myopathies are believed to result from an autoimmune reaction, whereby the body's own immune system attacks the muscle cells. Examples of inflammatory myopathies include polymyositis and dermatomyositis. Symptoms of polymyositis include muscle inflammation and muscle tenderness. The onset of symptoms may be acute, but the condition usually progresses slowly and, if left untreated, may compromise the subject's ability to walk. Subjects in need of treatment for dermatomyositis have similar symptoms as with polymyositis, but additionally show signs of a distinctive skin rash. Specifically, a violet-colored or dusky red rash breaks out over the subject's face, eyelids, and areas around their nails, knuckles, elbows, knees, chest, and back. Dermatomyositis typically occurs in adult subjects in their late 40s to early 60s or in children between the ages of 5 and 15.

The compositions and methods comprising nNOSμ fusion proteins can also be used to treat muscle atrophy. Muscle atrophy can be the result of a disorder or condition such as, for example, cancer cachexia, AIDS cachexia, or cardiac cachexia. Cachexia is generally associated with the massive loss (up to 30% of total body weight) of both adipose tissue and skeletal muscle mass that may occur as a side effect of many diseases such as cancer, AIDS, and chronic heart failure. The loss of adipose tissue and skeletal muscle mass can lead to anorexia, early satiety, fatigue, generalized muscle weakness, decreased muscle function, and progressive muscle wasting. Muscle atrophy can also be induced by the loss of innervation or damage to innervation of the muscle tissue. Specifically, diseases such as chronic neuropathy and motor neuron disease can cause damage to innervation. Moreover, many times a physical injury to the nerve can lead to damage to the innervation of the muscle tissue.

Alternatively, muscle atrophy can be the result of environmental conditions such as during spaceflight or as a result of aging or extended bed rest. Under these environmental conditions, the muscles do not bear the usual weight load, resulting in muscle atrophy from disuse.

The phrase “effective amount” or “therapeutically effective amount” of an nNOSμ fusion protein or derivative described herein is the minimum amount necessary to, for example, increase or improve one or more muscle function parameters, such as, for example, contractility, fatigue, and/or muscle damage. Accordingly, the “therapeutically effective amount” to be administered to a subject is governed by such considerations, and, as used herein, refers to the minimum amount necessary to prevent, ameliorate, treat, or stabilize, a neuromuscular disorder or condition as described herein. In some embodiments, the effective amount is sufficient to improve muscle function by at least about 5%, e.g., by at least about 10%, at least about 20%, at least about 30%, at least about 40%, at least about 50%, at least about 60%, at least about 70%, at least about 80%, at least about 90%, at least about 95%, at least about 96%, about 98%, about 99%, or 100%, as compared to muscle function in the absence of the nNOSμ fusion protein or derivative.

In some embodiments, the effective amount is sufficient to reduce neuromuscular morphologies that occur in muscle cells. Various established in vitro and in vivo assays can be used to determine an effective amount of the nNOSμ fusion protein or derivative for inhibiting neuromuscular pathology in muscle cells, as described, for example, in the Examples. Exemplary measurable responses are muscle contractility and/or muscle fatigue.

Accordingly, in some embodiments, the effective amount of the nNOSμ fusion protein is sufficient to increase muscle contraction and/or decrease muscle fatigure by at least about 5%, e.g., by at least about 10%, at least about 20%, at least about 30%, at least about 40%, at least about 50%, at least a bout 60%, at least about 70%, at least about 80%, at least about 90%, at least about 95%, at least about 96%, about 98%, about 99%, or 100%, as compared to the absence of the nNOSμ fusion protein.

By “reduce” or “inhibit” in terms of the neurodegenerative disorder treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom of a neurodegenerative disorder.

In some embodiments, the effective amount of an NOSμ fusion protein is about 0.1 mg/kg to about 100 mg/kg. In some embodiments, the effective amount of an NOSμ fusion protein can be present in an amount of about 0.5 mg/kg to about 100 mg/kg, about 1 mg/kg to about 75 mg/kg, about 3 mg/kg to about 50 mg/kg, about 5 mg/kg to about 25 mg/kg, or about 5 mg/kg to about 15 mg/kg. In some embodiments, the effective amount of an NOSμ fusion protein is about 10 mg/kg.

In some embodiments, the effective amount of an NOSμ fusion protein is about 5 nM to about 1 M. In some embodiments, the effective amount of an NOSμ fusion protein can be present in an amount of about 5 nM to about 5 μM, about 5 nM to about 100 μM, about 5 nM to about 500 μM, about 5 μM to about 1 mM, or about 1 mM to about 1 M.

### Subjects and Selection of Subjects

In some embodiments of the compositions and methods comprising nNOSμ fusion proteins for the treatment of subjects having a neuromuscular disorder, the subject to be treated is first selected. The determination as to whether a subject has a neuromuscular disorder, such as a muscular dystrophy, as well as the determination of a particular type of muscular dystrophy, can be made by any measure accepted and utilized by those skilled in the art. For example, diagnosis of subjects with muscular dystrophy is generally contingent on a targeted medical history and examination, biochemical assessment, muscle biopsy, or genetic testing.

In some embodiments, a subject's medical history can be used to diagnose or select a subject having a muscular dystrophy. Subjects with Duchenne muscular dystrophy, for example, are symptomatic before the age of 5 years, and experience difficulty running, jumping, and climbing steps. Proximal weakness causes individuals to use their arms in rising from the floor (i.e., Gowers' sign). Independent ambulation is often lost by 14 years of age, with subsequent deterioration in respiratory function and development of contractures and scoliosis. (See, e.g., Darras (2006) Continuum. 12: 33-75). Static cognitive impairment is common (Wicksell et al. (2004) Dev Med Child Neurol. 46:154-159). Approximately one third of boys with Duchenne muscular dystrophy develop cardiomyopathy by 14 years of age, and virtually all do after 18 years. Congestive heart failure and arrhythmias are common in end-stage Duchenne muscular dystrophy. (See, e.g., Kirchmann et al. (2005) Pediatr Cardiol. 26:66-72). Most young men with Duchenne muscular dystrophy die in their late teens or early twenties from respiratory insufficiency or cardiac failure.

In some embodiments, biochemical assessments can be run to determine the levels of various constituents of muscle and muscle fibers, such as, for example, measurement of blood or serum creatine kinase levels, can be used to diagnose or select a subject having muscular dystrophy. Specifically, when the total CPK level is substantially elevated, it usually indicates injury or stress to one or more of the heart, brain, and skeletal muscle. Before the age of 5 years, serum creatine kinase levels are 10 to 200 times higher in subjects with Duchenne muscular dystrophy and Becker muscular dystrophy compared to normal levels. (See, e.g., Cardamone et al. (2008) Semin Neurol. 28:250-9). In some embodiments, a subject in need of treatment generates less electrical activity during muscle contraction as compared to a healthy subject and this can be detected by electromyography. In other embodiments, subjects affected by either Duchenne muscular dystrophy or Becker muscular dystrophy, can be diagnosed by measuring the level of dystrophin. Typically, in subjects with either Duchenne muscular dystrophy or Becker muscular dystrophy, the level of dystrophin is deficient; but, in a subject with Duchenne muscular dystrophy, the level is more severely deficient. Specifically, many Duchenne muscular dystrophy patients are null for dystrophin expression resulting from an out of frame deletion.

In some embodiments, muscle biopsy can be used to diagnose or select a subject as having muscular dystrophy. For example, muscle biopsy from Duchenne muscular dystrophy patients shows degeneration, regeneration, and variability of fiber size with replacement of muscle by fat and connective tissue. Muscle immunohistochemical studies with anti-dystrophin antibodies shows complete absence of staining in muscle from subjects with Duchenne muscular dystrophy and reduced staining in muscle from subject with Becker muscular dystrophy. (Cardamone et al. (2008) Semin Neurol. 28:250-9).

In some embodiments, genetic testing can also be employed to diagnose or select a subject as having muscular dystrophy or a neuromuscular disorder. Techniques used in genetic testing include the polymerase chain reaction (PCR), Southern blotting, mutation scanning, and/or sequence analysis. (See, e.g., Darras (2006) Continuum. 12: 33-75). DNA extracted from blood or white cells can be used for such diagnoses. Deletions in the dystrophin gene are detected in 65% of patients with Duchenne muscular dystrophy and 85% of patients with Becker muscular dystrophy. Quantitative assays of dystrophin can be used to predict phenotype. Patients with Duchenne muscular dystrophy, for example, have less than 5% of the normal quantity of dystrophin. Patients with Becker muscular dystrophy have at least 20% normal dystrophin levels. (See Cardamone et al. (2008) Semin Neurol. 28:250-9).

In some embodiments, magnetic resonance imagining (MRI) can also be employed to diagnose or select a subject as having muscular dystrophy or a neuromuscular disorder. During an MRI, cross-sectional images of muscle are generated by a magnetic field and radio waves. The image generated by an MRI can reveal abnormalities in the muscle, such as inflammation, damage, or infection.

The term “muscle function” refers to the ability of muscle to perform a physiologic function, such as contraction as measured by the amount of force generated during either twitch or tetanus. Other methods for assessing muscle function are well known in the art and include, but are not limited to, measurements of muscle mass, grip strength, serum CK level, activities of daily living, motion or strength tests, tissue histology (e.g., E&A staining, or collagen III staining), or tissue imaging. Nonlimiting illustrative methods for assessing muscle function are also set forth in the Examples.

### EXAMPLES

### Material and Methods

The work described herein includes the generation and primary characterization of new transgenic mice expressing nNOSμ and restoring its sarcolemmal localization. We used molecular biology tools to clone nNOSμ. In order to restore cytoplasmic nNOSμ expression, a nNOSμ transgene was expressed only in the skeletal muscles of mdx mice; because of the absence of dystrophin in mdx mice, the nNOSμ protein cannot target to the sarcolemma, but only to the cytoplasm. On the order hand, to achieve sarcolemmal localization of transgenic nNOSμ in the mdx context, nNOSμ carrying the k-Ras plasma membrane targeting sequence was expressed in mdx skeletal muscles. This construct produces palmitoylated nNOSμ which targets to the sarcolemma, even in the absence of dystrophin. A set of physiological, cell biology and biochemical tools were used to evaluate transgene expression and impact on mdx skeletal muscle pathology and function.

The knowledge of the physiological functions of nNOSμ and their dependence on subcellular localization provide insight into nNOSμ splice variant function in dystrophic muscle. Furthermore, these studies reveal the usefulness of targeting nNOSμ signaling pathways for therapeutic intervention in muscular dystrophies including DMD and other diseases where nNOSμ signaling is defective.

nNOSμ Cloning.

Total RNA was extracted from mouse skeletal muscle using the RNEASY FIBROUS TISSUE MINI KIT (QIAGEN). The RNA extracted was used as template to obtain total cDNA using an oligo-dT and SUPERSCRIPT III FIRST-STRAND SYNTHESIS SYSTEM for RT-PCR (INVITROGEN). nNOSμ was amplified by PCR (ACCUPRIME Pfx DNA Polymerase (INVITROGEN) from total cDNA of mouse skeletal muscle using primers nnos5b and nnos3b. A second round of PCR was made with primers designed to insert restriction sites for NotI (5′) and PacI (3′) (nos5aNot, nos3aPac). List of primers are listed in Table 1. As cloning vector, we used PBLUESCRIPT KS-, which has ampicillin resistance and works to perform α-Complementation screening (Sambrook, 2001). PBLUESCRIPT vector was digested with NotI (NEB) (producing protruding ends) and EcoRV (NEB) (producing blunt ends), while the nNOS PCR product was digested with NotI. The DNA fragments from the restriction reaction were separated by electrophoresis, and the desired bands were gel purified with the QIAQUICK Gel Extraction Kit (QIAGEN) and ligated with T4 DNA ligase (NEB) (16° C., ON) into PBLUESCRIPT KS-vector. The ligation mixture was used to transform electro- or chemically competent E. coli DH5α. The transformed bacteria suspension was spread in agar plates supplemented with 100 μm/mL ampicillin, 20 ug/mL X-gal and 0.1 mM IPTG. Individual white colonies were picked with a sterile toothpick or pipette tip to inoculate 14 mL falcon tube containing 2 mL of LB media, which was shaked at 37° C. ON. The plasmid DNA was purified with a Rapid miniprep protocol, as is described below.

Rapid Miniprep:

Centrifuge the bacteria suspension in a 1.5 mL eppendorf tube at maximum speed in a table top centrifuge. Discard the supernatant by inverting the tube. Resuspend the pellet in the remaining LB media. Add 200 uL of lysis solution (25 mM Tris-HCl pH=8; 10 mM EDTA pH=8; 0.5N NaOH; 0.2% SDS) and mix using vortex. Add 100 uL of 5M Sodium Acetate (pH=5) and vortex. Centrifuge at maximum speed for 10 minutes. Transfer the supernatant to a new 1.5 mL eppendorf tube, add 750 μL of 100% ice cold ethanol and centrifuge 5-10 minutes at maximum speed. Wash the pellet with 500 μL of 70% ice cold ethanol and centrifuge for 5 minutes. Let dry the pellet and resuspend it in TE buffer (100 mM Tris-Cl pH=7.5; mM EDTA pH=7.5). The plasmid DNA was analyzed by restriction assay using BglII enzyme (NEB). The positive clones showed a characteristic 4 bands pattern. From several procedures, more than 500 colonies were screened, and the positives clones were less than 10. The positives clones were analyzed by PCR to contain μ- or α-nNOS isoform cDNA (mup1 and mup2 primers). This primer pair generates a band of 600 pb for nNOSα and 700 bp for nNOSμ. Recombinant plasmid carrying the full length nNOSμ cDNA were found, and some of them were sequenced (Department of Biochemistry, University of Washington). The software SEQUENCHER 4.9 was used to assemble the sequence, and the consensus from this contig was compared with the nNOSμ sequence (Silvagno et al., 1996). The clones showing no differences with the published sequences were chosen.

HA and HA-kRAS Tag Insertion.

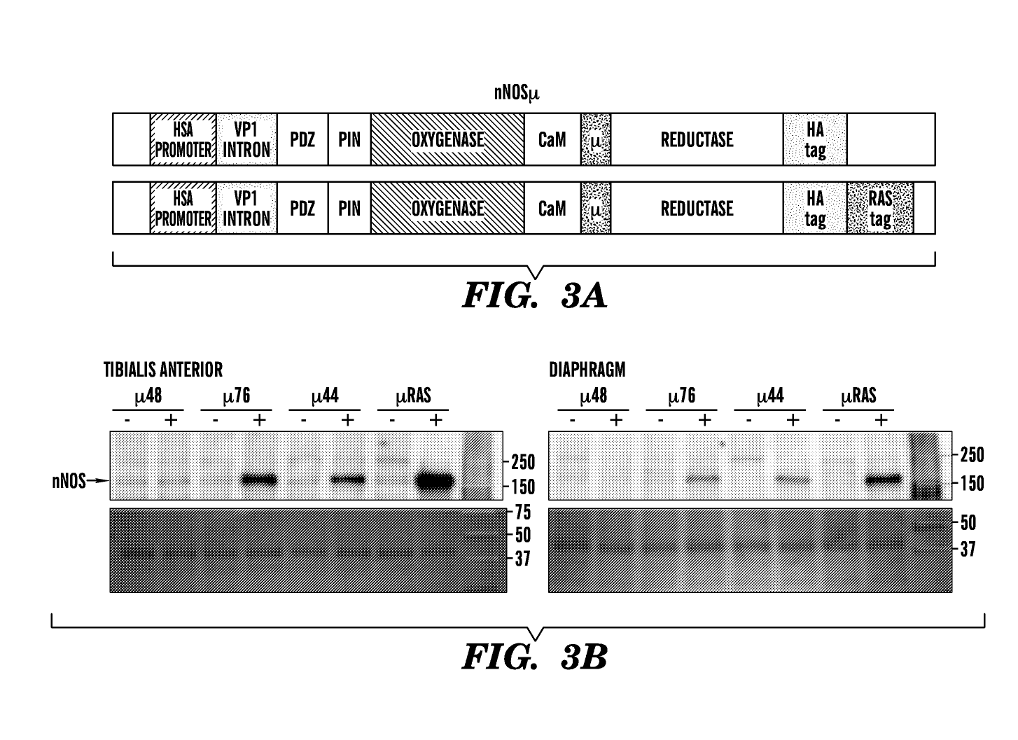

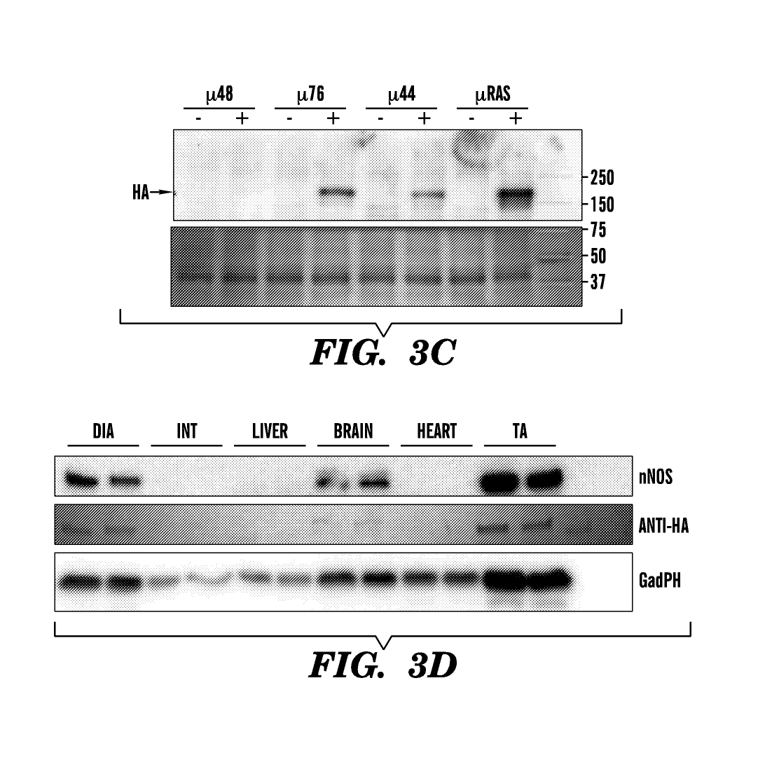

To obtain nNOSμ with an HA-tag, two oligos (HA1 and HA2) were designed to delete the original stop codon of nNOS and add the HA tag followed by a stop codon and a PacI restriction site. A PCR mix containing oligos HA1, HA2, Forward 6 and total cDNA as a template was used to obtain the 3′ nNOS cDNA carrying the HA tag. See FIG. 3 for a scheme of the procedure. To obtain the HA-k-RAS tag, the sequence containing the HA plus the k-Ras (plasma membrane targeting sequence from k-Ras protein amino acid sequence N-KDGKKKKKKSKTKCVIM-C) (SEQ ID NO: 6)) tag was amplified from a dystrobrevin-HA-kRas construct. The PCR products from both constructs were cloned in the NotI/PacI digested pBSX-HSAvpA vector (Crawford et al., 2000) and selected by ampicillin resistance. The nNOSμ transgene is downstream of the 2.2 kb human skeletal actin (HSA) promoter. A VP1 intron sequence from the SV40 virus, containing a transcriptional enhancer, is immediately downstream of the promoter to ensure high transgene expression. Use of the HSA promoter ensures skeletal muscle-specific expression of nNOSμ transgenes. The linearized vector were injected into zygotes of wild type (C57/Bl6) mouse, and wild type founder mice (C57/Bl6) expressing the nNOSμ transgene.

In Situ Analysis of Skeletal Muscle Contractile Function.

Mice were anesthetized with intraperitoneal injections of AVERTIN (2,2,2, tribromoethanol; SIGMA, St Louis, Mo.). Mouse hindlimbs are shaved and the distal TA tendon of the tibialis anterior (TA) muscle is surgically isolated via a skin incision on the anterior surface of the lower hindlimb. Then, the mouse is positioned on a 37° C. heated platform in order to restrain the knee joint and the distal tendon is attached to the lever arm of a servomotor (Model 305B-LR, AURORA SCIENTIFIC, ON, Canada). The exposed surface of the muscle is kept moist by frequent application of prewarmed isotonic saline. The TA muscle is stimulated by electrical excitation of the sciatic nerve using two needle electrodes. The muscle is adjusted to an optimum length (Lo) to produce the maximum tetanic force. Then, the time to reach peak tension (TPT) during the contraction phase of the twitch, and the half-relaxation time (HRT), the time between maximum and half maximum force during the relaxation phase of the twitch are recorded. While held at Lo, the TA is stimulated every two minutes at increasing frequencies (10 to 200 Hz) to generate force frequency curves, to obtain the maximal tetanic force (Po). After the completion of testing, both Lo and TA mass should be recorded and used to normalize forced for TA muscle weight and calculate specific twitch (Twitch SpF) or specific tetanic force (SpF). For each mouse, the first hindlimb is used for testing resistance to exercise-induced fatigue, while the second is used to test susceptibility to contraction-induced injury. At the conclusion of contractile function analysis, animals are sacrificed and the tibialis anterior (TA) diaphragm muscles are rapidly excised, weighed and frozen for analysis.

Resistance to Exercise-Induced Fatigue.

To test the capacity of muscle to sustain force output, TA muscles are subjected to a series of repeated contractions to simulate exercise and cause fatigue. Muscles are subject to maximal stimulation (40 V, 200 Hz) at 2 s intervals for 4 minutes. Maximum isometric force production was recorded every 2 s. Recovery from fatigue is assessed by recording maximal tetanic force output at 1 minute and 15 minutes after the completion of the fatigue period.

Resistance to Stretch Contraction-Induced Injury.

This parameter is assessed by subjecting TA muscles to a series of consecutive lengthening (stretch) contractions of progressively increased strain. Strain is the percentage increase in length beyond the optimal muscle length Lo. Muscles are maximally stimulated (4 V, 200 Hz) for 150 ms at fixed length to achieve maximal isometric tension, immediately followed by 200 ms of stimulation during the application of a length change of 20% beyond Lo. Strain is applied at the rate of 2 fiber lengths/s. Lengthening contractions are performed at 1 minute interval to minimize the impact of fatigue on force-generating capacity. The isometric SpF generated immediately prior to the initiation of the subsequent lengthening contraction is recorded and normalized.

In Vitro Analysis of Diaphragm Muscle Function.

Diaphragm muscle strips, 2 to 3 mm wide, are dissected in physiological buffer, bubbled with 95% O₂ and 5% CO₂ (pH 7.4). The diaphragm strip is then placed in the experimental chamber, which is continuously perfused with solution. The central tendon of the strip is attached, via a metal clip, to the lever of a dual-mode force transducer/length controller (AURORA SCIENTIFIC). At the other end of the chamber, the strip is attached to a stainless steel hook, via a small hole in the rib bone. Muscle stimulation is provided by two platinum electrodes, attached to the inside walls of the chamber, which are connected to a stimulator (AURORA SCIENTIFIC). Supramaximal stimulus voltage is set at 20% greater than the voltage required for maximum twitch force. A length-force curve is then measured by tetanic contractions (120 Hz, 300 ms duration), spaced 1 min apart, over a range of muscle lengths (from short to long). The optimum length (Lo) is the length at which maximum tetanic force is generated. Muscle fiber length (Lf) at Lo is then measured using calipers, for later calculation of specific force (force normalized to muscle cross-sectional area). At this stage, the muscle is subjected to fatigue protocol. For fatigue, the muscle is stimulated at 1 Hz for 60 seconds. Recovery of force following fatigue is measured at 1 min intervals up to 15 min.

Western Blotting (WB).

Equal amounts of protein extracts from transgenic mice are loaded on polyacrylamide gels to perform electrophoresis in presence of SDS (SDS-Page). After electrophoresis, proteins are transferred to a PVDF membrane. The nNOSμ protein, the HA tag and fibronectin were detected with incubation with primary antibodies at 4° C. over night (ON), followed by 3 washes, 10 minutes each, with TBS-T, and then incubation with HRP-conjugated antibodies.

Centronucleation and Myofiber Cross-Sectional Area (CSA) Calculations.

Freshly isolated mouse TA and diaphragm muscles are isolated from adult (8-10-week-old) mice, embedded in TISSUE TEK OCT compound and flash frozen in liquid nitrogencooled isopentane. Cryostat sections (10 μm) from the muscle mid-belly are stained with Wheat Germ Agglutinin (WGA-488) and DAPI.

Immunohistochemistry (IHC).

Muscles from 8- to 9-week-old mice are dissected, embedded in TISSUE TEK OCT, frozen in liquid nitrogen-cooled isopentane and then stored at −80° C. Cryostat sections (10 μm thick) are fixed in 2% paraformaldehyde for 10 min. Commercially available antibodies are used to detect proteins of interest.

Skeletal muscle fiber typing. To characterize the fiber composition of skeletal muscles, 10-μm-thick frozen sections were immunolabeled with mouse monoclonal antibodies raised against type I (BA-D5), type IIa (SC-71), and type IIb (BF-F3) MyHC proteins (Developmental Studies Hybridoma Bank). Sections are incubated ON at 4° C. with primary antibodies followed for 2-hour incubation with Alexa Fluor 350-labeled donkey anti-mouse IgG2B (BA-D5), Alexa Fluor 594 donkey anti-mouse IgG1 (SC-71), and Alexa Fluor 488 donkey anti-mouse IgM (BF-F3) isotype-specific secondary antibodies. The frequency of MyHC type I, type IIa, type IIb, and type IIx/IId fibers per section are counted manually. Unlabeled fibers are designated type IId/IIx. The area of the muscle sections and the area of MyHC are determined using IMAGE J V1.38X software.

nNOSμ Cloning and Transgenic Mice Generation.

nNOSμ cDNA was derived from total skeletal muscle RNA using reverse transcription-PCR and the presence of the mu insert confirmed by sequencing. We used PCR to add a single heme-agglutinin (HA) tag epitope to the C-terminal of nNOSμ or the HA tag preceded by the palmitylation signal sequence from the K-ras oncogene (amino acid sequence: KDGKKKKKKSKTKCVIM (SEQ ID NO: 6)) (Kahn et al., 1987). These two nNOSμ constructs were cloned into the NotI/PacI sites of pBSX-HSAvpA vector (Crawford et al., 2000). The pBSX-HSAvpA vector contains the human skeletal actin (HSA) promoter that provides skeletal muscle-specific expression of the nNOSμ transgenes. Linearized constructs were injected into oocytes of a cross of the C57BL/6 and C3H mouse strains (Transgenic Resources Program, University of Washington). Lines of mice expressing the transgenes were bred onto C57bl10 and mdx backgrounds for a minimum of 3 generations. All experimental procedures performed on mice were approved by the Institutional Animal Care and Use Committee at the University of Washington.

### In Situ Analysis of Skeletal Muscle Contractile Function.

Anesthetized mice were positioned on a 37° C. heated platform, the distal tendon of the Tibialis anterior (TA) muscle was surgically isolated, the knee joint was restrained with a surgical needle and the distal tendon attached to the lever arm of a servomotor (Aurora Scientific, ON, Canada). The exposed surface of the muscle was kept moist by frequent application of pre-warmed isotonic saline. The TA muscle was stimulated by electrical trigger of the sciatic nerve using two needle electrodes. The muscle was adjusted to an optimum length (Lo) to produce the maximum tetanic force. While held at Lo, the TA was stimulated every two minutes at increasing frequencies (10 to 200 Hz) to generate force frequency curves, to obtain the maximal tetanic force (Po). Then, both L0 and TA mass were recorded and used to normalize to the physiological cross sectional area ([L0×density]/mass) and calculate specific tetanic force (SpF). For each mouse, the left TA was used for testing resistance to exercise-induced fatigue, while the right was used to test susceptibility to contraction-induced injury. To test Resistance to Exercise-Induced Fatigue, TA muscles were subjected to a series of repeated maximal stimulations (200 Hz) at 2 sec intervals for 4 min, while maximum isometric force production was recorded. Recovery from fatigue was assessed by recording force output every 2 minutes for at least 15 minutes after the completion of the fatigue period.

### Resistance to Eccentric Contraction (ECC)-Induced Injury.

TA muscles were subjected to a series of consecutive lengthening (stretch) of the same strain (20% beyond Lo). Muscles were maximally stimulated for 175 ms at fixed length to achieve maximal isometric tension, immediately followed by 175 ms of stimulation during stretching at the rate of 2fiber lengths/sec. ECC were performed at 1 minute interval and the isometric SpF generated in each round was recorded and normalized.

### In Vitro Analysis of Diaphragm Muscle Function.

Diaphragm muscle strips, 2 to 4 mm wide, were dissected in physiological buffer, bubbled with 95% O2 and 5% CO2 (pH 7.4). The central tendon of the strip was attached to the lever of a servomotor (Aurora Scientific) and the rib bone was attached to a stainless steel hook. Muscle stimulation was provided by two platinum electrodes, attached to the inside walls of the chamber, which are connected to a stimulator (Aurora Scientific). Supramaximal stimulus voltage was set at 20% greater than the voltage required for maximum twitch force. Lo was is the length at which maximum tetanic force is generated. Muscle fiber length (Lf) at Lo is then measured using calipers, for later calculation of specific force (force normalized to muscle physiological cross-sectional area). At this stage, the muscle is subjected to a fatigue protocol. For fatigue, the muscle is stimulated at 120 Hz, every second for 60 seconds. Recovery of force following fatigue was measured at 1 min intervals up to 15 min.

### Antibodies

Antibodies to utrophin, a-syntrophin, and a-dystrobrevin have been described previously (peters 97). We used the following commercial antibodies. Rabbit polyclonal antibodies; glyceraldehyde-3-phosphate dehydrogenase (Millipore), Fibronectin (Sigma), nNOS (Invitrogen), aquaporin-4 (Millipore), HA (Sigma). Mouse monoclonal antibodies; pan dystrobrevin (BD Biosciences) and b-dystroglycan (Novacastra). Goat polyclonal anti HA (Abcam).

### Western Blotting (WB).

Muscle extracts in 1% triton were prepared as described previously (Peters 2 syntrophin). Protein concentrations were determined using the bicinconic acid reagent (Pierce) and equal amounts of muscle protein were used for SDS-PAGE. Proteins were transferred to PVDF membrane (Millipore) and the primary antibodies detected using HRP-conjugated secondary antibodies (Jackson Immunoresearch) and ECL substrate (Pierce) with a Protein Simple Fluorochem M imager.

### Immunohistochemistry (IHC).

Muscles from 8- to 9-week-old mice were dissected, embedded in Tissue Tek OCT, frozen in liquid nitrogen-cooled isopentane. Cryostat sections (10 μm thick) were incubated with primary antibody 4° C. overnight then with ALEXA FLUOR-conjugated secondary antibodies (Invitrogen) for 1 h at room temperature. Images were obtained using a Leica TCS-NT confocal microscope at the W.M. Keck Center for Advanced Studies in Neural Signaling at the University of Washington.

### Data Analysis.

In all experiments, transgenic mice were compared to non-transgenic littermates. As different lines were obtained, and results from different lines for the same transgene performed very similar, we used pooled data. Thus, C57Bl10 and mdx non transgenic data correspond to negative littermates of all transgenic lines. Data from cytosolic nNOSμ transgenic mice corresponds to the average of 3 different lines. Values are expressed as Mean±SEM, and statistical differences were evaluated with Prism 5 Software, using One-way ANOVA with Bonferroni multicomparison post-test to evaluate differences.

### Generation of Transgenic Mice

Transgenic expression of nNOSα in the mdx mouse has been reported to improve the histopathology of skeletal muscle (Wehling et al., 2001). However, as described herein, these studies have several limitations, the most important being the failure of the enzyme to target to the sarcolemma and the use of nNOSα, the brain isoform rather than the muscle-specific nNOSμ. We have taken a different approach to achieve a more physiological distribution of nNOSμ in skeletal muscle.

In order to obtain transgenic mice, the cDNA of nNOSμ, and the modified nNOSμ-RAS (carrying the palmitoylation signal from the K-Ras oncogene) were cloned into the pBSX-HSAvpA vector (Crawford et al., 2000), where protein expression is driven by the HSA-promoter, which is active early in embryonic muscle development (FIG. 3A). A hemagglutinin tag (HA) was also added. To obtain different lines of the two constructs, several pronuclear injections were performed. We detected positive founders for nNOSμ and nNOSμ-RAS transgene by PCR. Founders were crossed with C57Bl10 and mdx (same genetic background) mice and the first generation pups (N1) were genotyped using PCR to analyze the presence and transmissibility of the transgene. Some of the founders bred successfully, obtaining about 50% of positives pups. Positives pups were divided in two groups. The first group was kept in order to continue the backcrossing with C57Bl10 or mdx until the 3^{rd} generation (N3). The second group was sacrificed at 4-6 weeks old to analyze whether the transgene was expressing the encoded protein. We used western blot to analyze Tibialis anterior and Diaphragm muscle homogenates, and evaluated nNOS protein expression using an anti-NOS antibody (FIG. 3B). To detect only the transgenic protein, an anti-HA antibody was used, since both transgenes products carry the HA tag. (FIG. 3C). Several lines of transgenic mice with different levels of expression were obtained. Although pBSX-HSAvpA vector and the HSA promoter has been previously used and described to drive only skeletal muscle expression of protein (Crawford et al., 2000), a control western blot using protein homogenates from different tissues was performed to show muscle specific expression of the transgene (FIG. 3D).

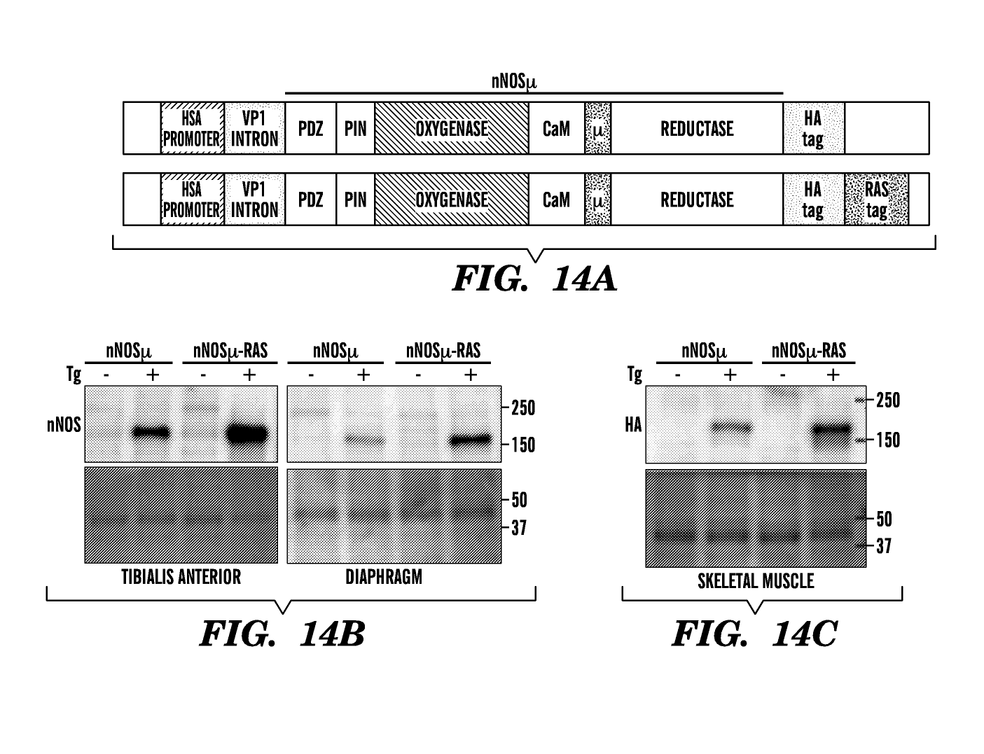

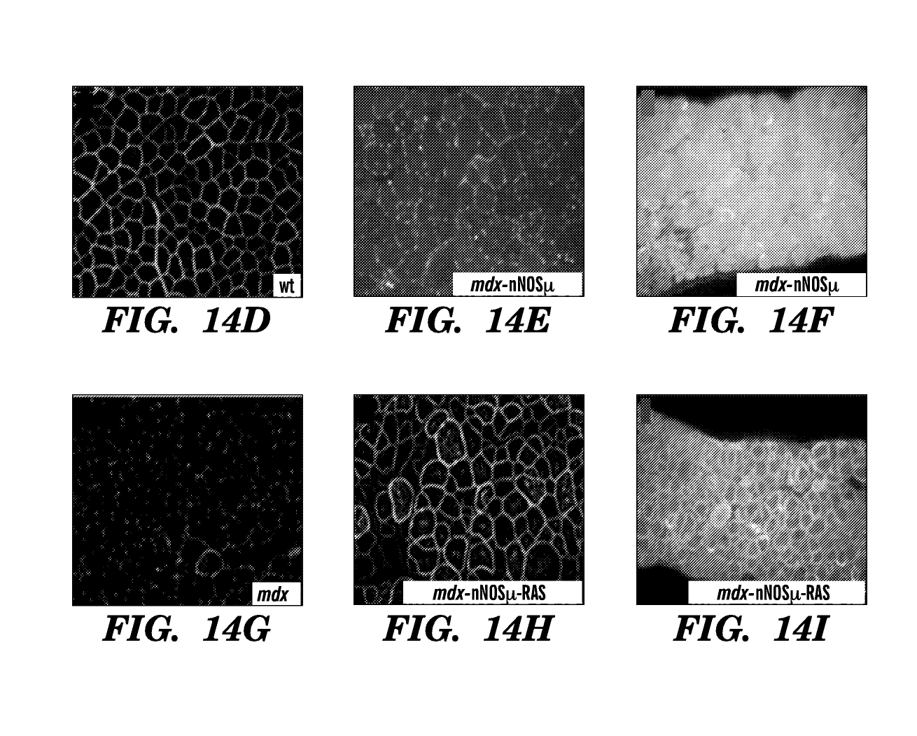

To compare the effects of general nNOSμ expression with sarcolemma targeted nNOSμ expression we generated two lines of transgenic mice. Both used the Human Skeletal Actin promoter to drive transcription (FIGS. 14A-14I). This promoter is only active in skeletal muscle and is active from early in embryonic muscle development to mature skeletal muscle (Crawford et al., 2000). A hemagglutinin tag (HA) was placed at the C-terminal end of the nNOSμ in both lines. In one transgenic line we added a C-terminal palmitoylation signal sequence from the K-Ras oncogene to target the nNOSμ to the sarcolemma. We obtained 3 founder mice from the TgnNOSμ line and 2 founders for the Tg nNOSμ-RAS line. Each founder line was bred onto the mdx background for at least 3 generations before obtaining data.

Both nNOSμ and nNOSμ-RAS expressed well in skeletal muscle (FIGS. 14A-14I). Western blots of tissue homogenates showed high expression of the nNOS transgene products in both TA and diaphragm muscles Immunofluorescence studies of these two muscles show that transgenicly expressed nNOSμ is only faintly labeled at the sarcolemma (FIGS. 14E-14F) while the nNOSμ-RAS shows robust sarcolemmal labeling (FIGS. 14H-14I).

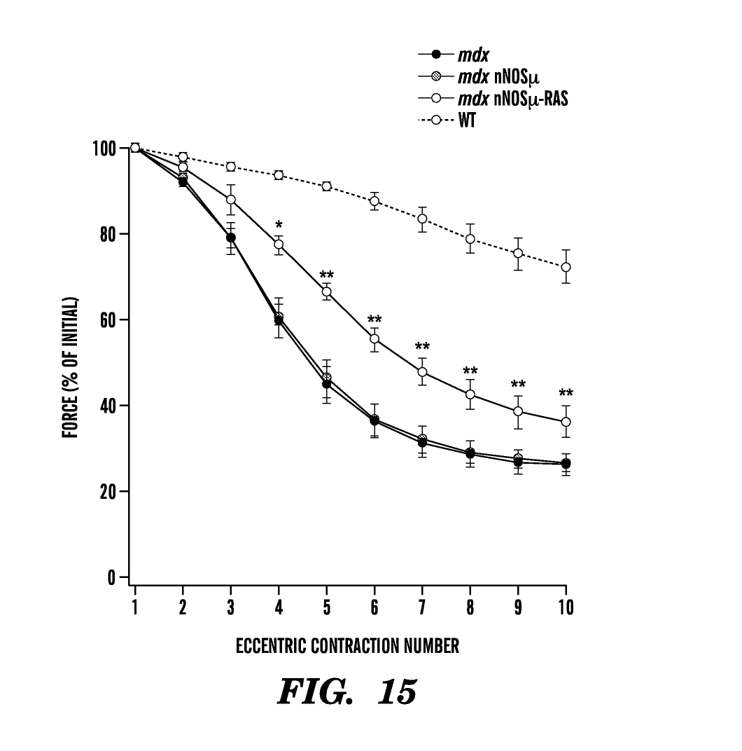

DMD and mdx muscle are particularly vulnerable to damage from eccentric contractions. We therefore tested the performance of skeletal muscle from control (C57bl10), mdx, and both transgenic lines (bred onto the mdx background) during eccentric contractions. These experiments were performed in vivo with the distal tendon of the TA muscle attached to a force transducer as described. Force was measured during sequential contractions initiated by direct stimulation of the sciatic nerve (FIG. 15). The reduction in force in the mdx mice is evident as early as the second contraction. After 5 contractions, the force generated in the mdx and mdx/nNOSm muscle has fallen to ˜40% of the initial force while in the mdx/nNOSm-RAS the force has only dropped to ˜60% of initial force (note wild type drops to ˜90% of initial force). While the sarcolemmal expression of nNOS does not completely restore the muscle force to that of wild type it does result in significant improvement of muscle performance.

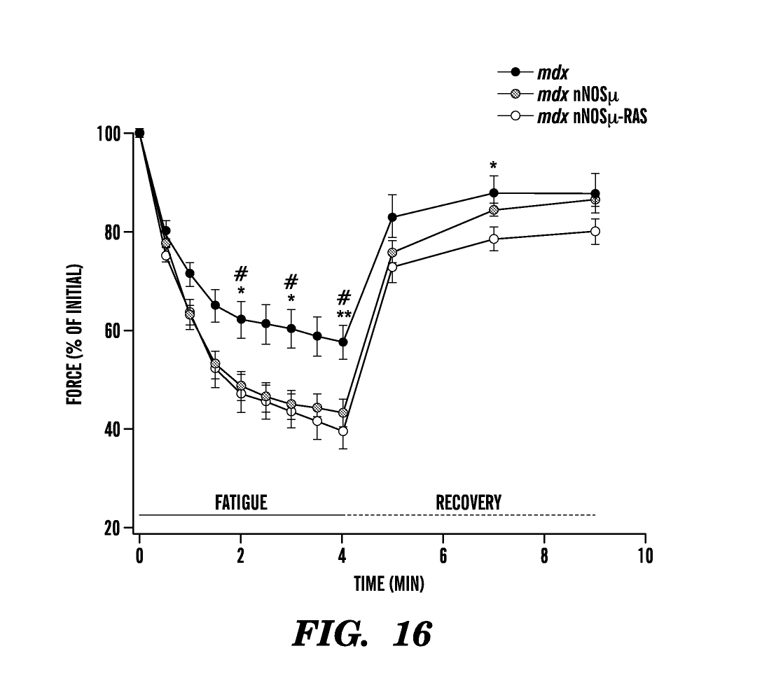

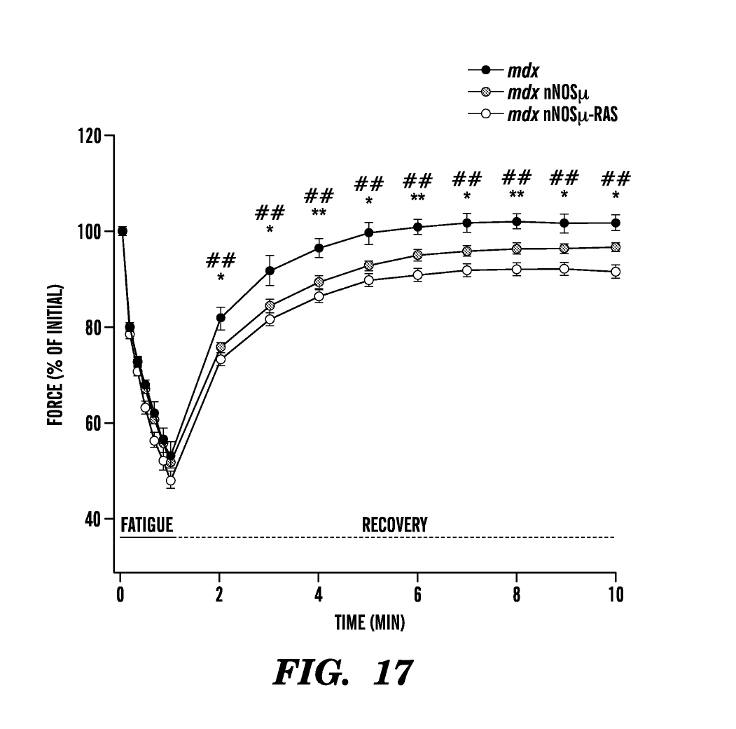

We also determined the effect of sarcolemmal nNOS restoration in skeletal muscle specific force and fatigue (FIGS. 16 and 17). The specific force of TA muscle was increased in the mice with sarcolemmal nNOS at low frequency (physiological) stimulation (FIG. 16). This muscle also showed substantially less fatigue and quicker recovery than either the mdx control or the transgenic/mdx mice that did not target nNOS to the sarcolemma. In diaphragm we did not see the increase in resistance to fatigue that was observed in the limb muscle in the sarcolemmal nNOS mice. However the diaphragm of the sarcolemmal nNOS mice did show a faster recovery from fatigue similar to that seen in limb muscle (FIG. 17).

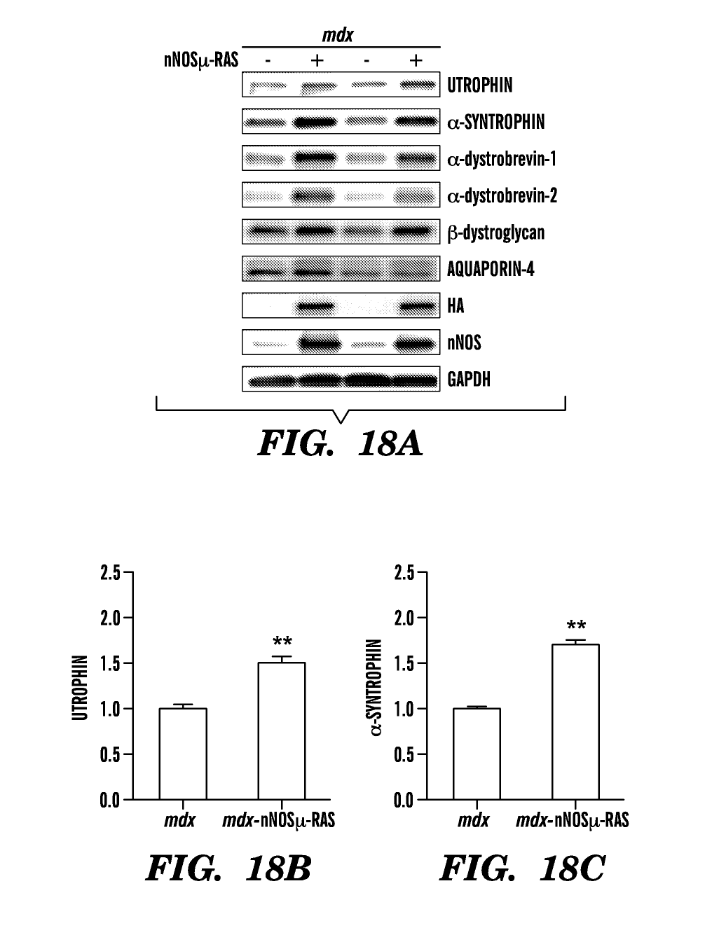

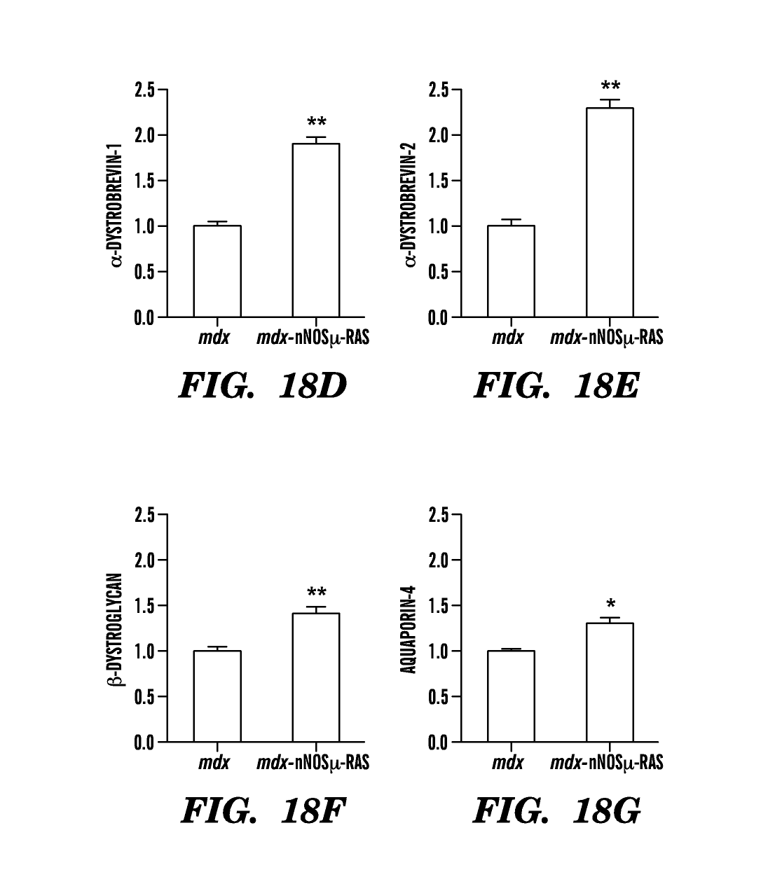

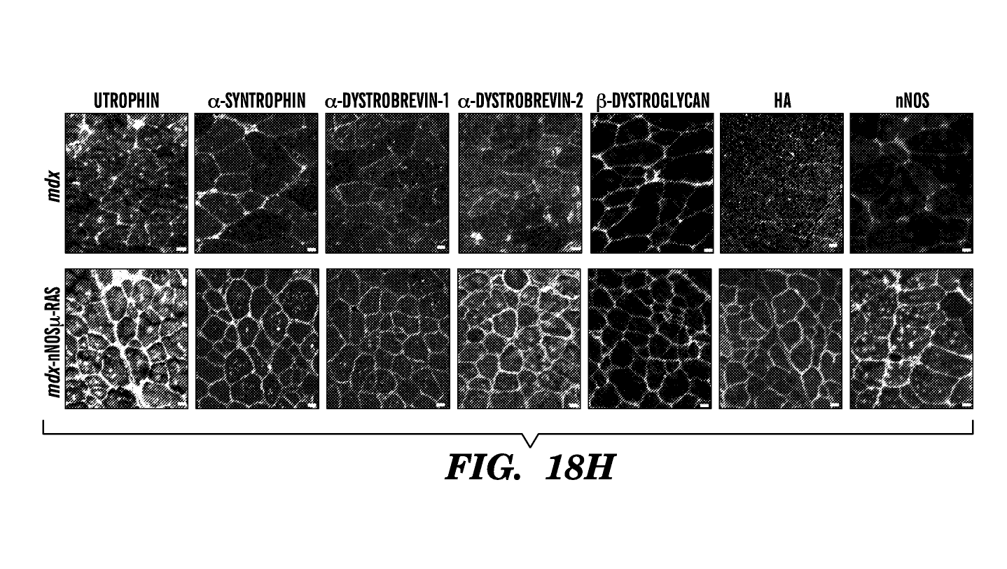

The physiological improvement we observed in mdx muscles expressing sarcolemmal nNOS prompted us to examine the levels of dystrophin related and/or associated proteins at the sarcolemma. We isolated muscle membranes quantitated the levels of dystrophin complex proteins using western blot analysis (FIGS. 18A-18FH). We found that mice expressing sarcolemmal nNOS had significant increases in many of the dystrophin complex proteins. These include utrophin, dystrobrevin, syntrophin, beta-dystroglycan, and aquaporin-4. While not wishing to be bound or limited by theory This increase in dystrophin complex proteins is likely the basis of the physiological improvement we observed in the sarcolemmal nNOS mice.

Localization of nNOSμ Transgenic Protein

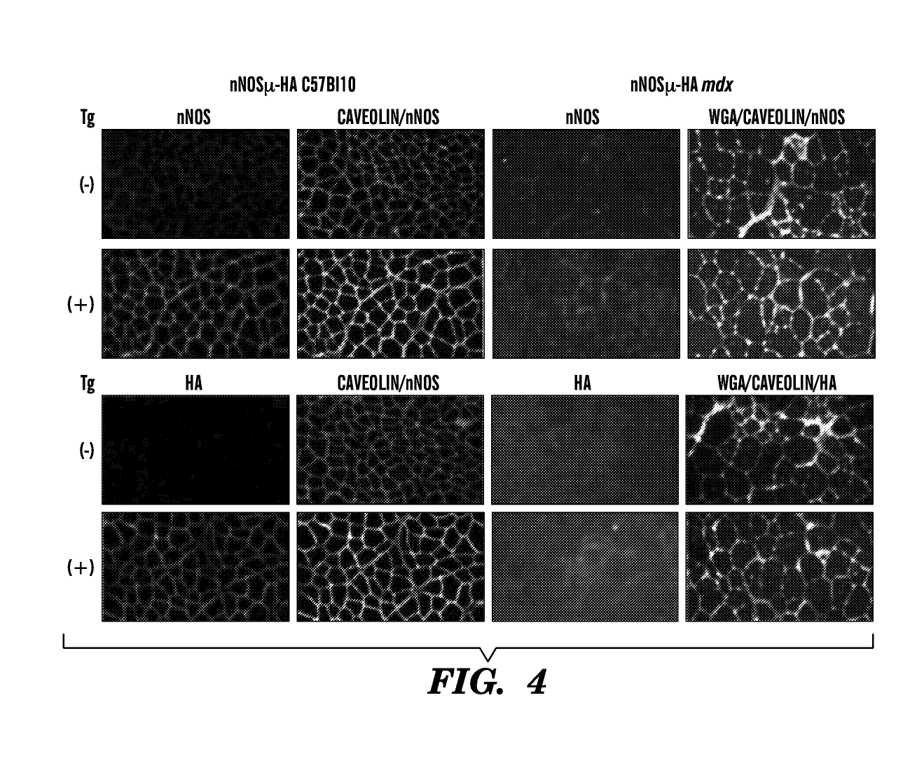

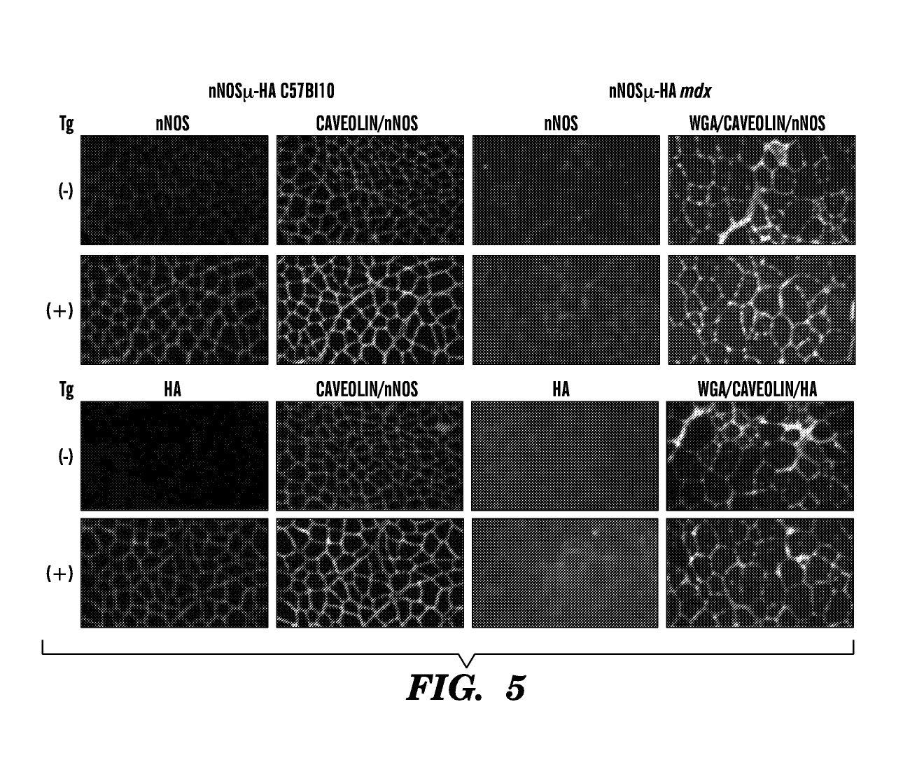

In order to evaluate localization of nNOSμ transgenic protein, we used 10 um sections of isopentane-frozen TA muscle for immunofluorescence microscopy. Staining for nNOS and the HA-tag shows that mice expressing unmodified nNOSμ can localize the enzyme to the sarcolemma in the wt C57Bl10 mice. As expected, no sarcolemmal staining is seen when the transgene is expressed in the mdx background (FIG. 4). However, as shown in FIG. 5, transgenic nNOSμ-RAS protein shows sarcolemmal localization in both wt C57Bl10 and mdx backgrounds. The same results, though with lighter staining, were observed in Diaphragm muscle.

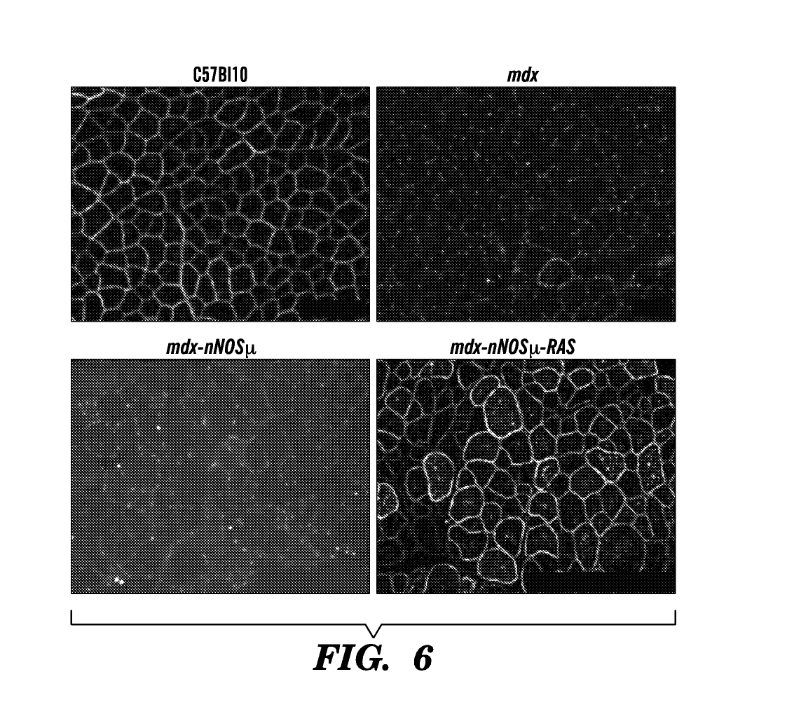

A summary panel for nNOSμ localization compares wt, mdx and transgenic lines. As shown in FIG. 6, wt C57Bl10 mice show endogenous nNOSμ on the sarcolemma, while mdx mouse sarcolemma lacks the enzyme. mdx mice expressing RAS-nNOSμ have robust amounts of the recombinant enzyme on the sarcolemma. In contrast, the sarcolemma of mdx mice expressing unmodified nNOSμ is devoid of the enzyme. Thus, addition of the RAS sequence to nNOSμ alleviates the requirement that both dystrophin and α-syntrophin are needed for sarcolemmal association. This is a novel and unprecedented achievement.

### Evaluation of Muscle Performance

We evaluated muscle function using two different approaches. First, we used an ex vivo evaluation of diaphragm muscle performance as described previously (Whitehead et al., 2008). The mdx diaphragm most accurately reflects the severity of the dystrophic phenotype in DMD (Stedman et al., 1991). Since failure of respiratory muscles is a major cause of death in DMD boys, any treatment that improves diaphragm function will likely have therapeutic value and effects on longevity and quality of life. However, we considered this approach could have some limitations when assessing nNOSμ function in skeletal muscle, given the important role of nNOSμ in regulating blood delivery during muscle contraction and the dependence of nNOS function on oxygen concentration (Eu et al., 2003; Thomas et al., 1998; Thomas et al., 2003). Even though controlled buffered solution is used to keep diaphragm strips metabolically active, the conditions, especially oxygen levels, are far from physiological environment. We evaluate muscle function by performing in situ contractile physiological analysis of Tibialis anterior (TA) muscle (Percival et al., 2008). The advantage of in situ analysis is that it allows for measurement of contractile properties with the muscle in its natural environment, maintaining normal vasoregulation and innervation.

We measured different parameters of muscle performance. In both, diaphragm ex vivo and TA in situ techniques, we measured Specific Force (SpF). SpF is the maximum tetanic force the muscle can develop in its optimal length (L₀), normalized to the physiological cross sectional area ([L₀×density]/weight). When calculating SpF in penniform muscles (those in which muscle fascicles attach obliquely to the tendon) as the TA, L₀ must be corrected by the pennation angle. Pennation angle is formed by the individual muscle fibers with the line of action of the muscle, and the used value is an average for the entire muscle, which in TA correspond to a value of 0.6 (Hakim et al., 2011). At the same L₀, twitch Specific force (SpF generated after a single stimulation) was measured. From these records, parameters as the time to reach peak tension (TPT) during the contraction phase of the twitch and the half-relaxation time (HRT), the time between maximum and half maximum force during the relaxation phase of the twitch, can also be analyzed as a measurement of the kinetics of the muscle contraction.

To evaluate muscle function during activity, we performed a protocol that pretends to mimic what happens, for example, during exercise. This protocol is used to evaluate muscle fatigue susceptibility, and was performed in both Diaphragm and TA muscles (see Methods) (Percival et al., 2008). Muscles are subjected to consecutive tetanic stimulations every 1 or 2 seconds; muscle fatigues and force decreases after each contraction. Then, tetanic stimulations are performed every 1 minute for at least 10 minutes, to evaluate muscle force recovery.

### Ex Vivo Evaluation of Diaphragm Performance

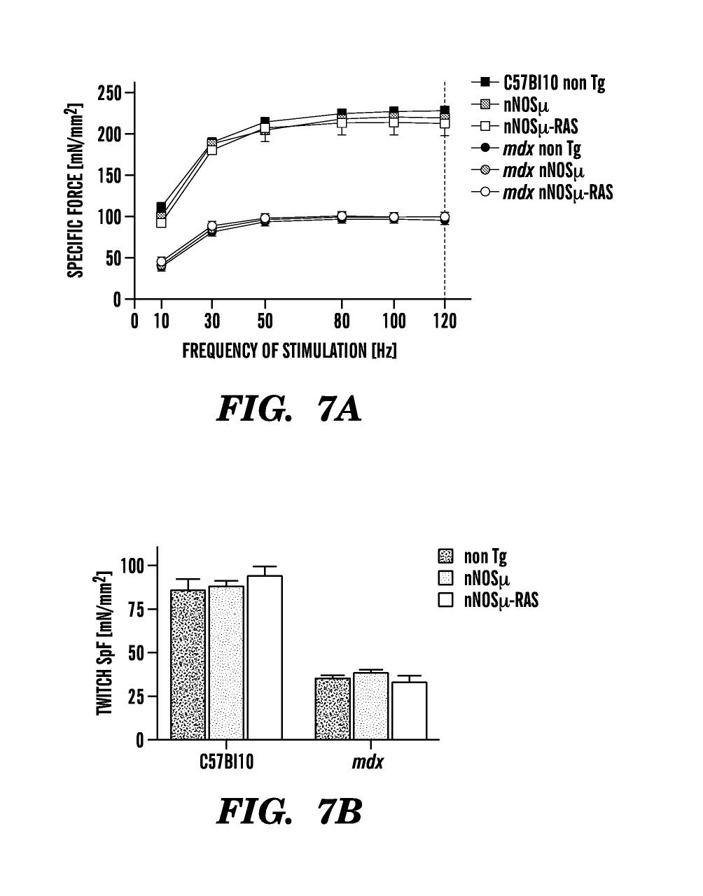

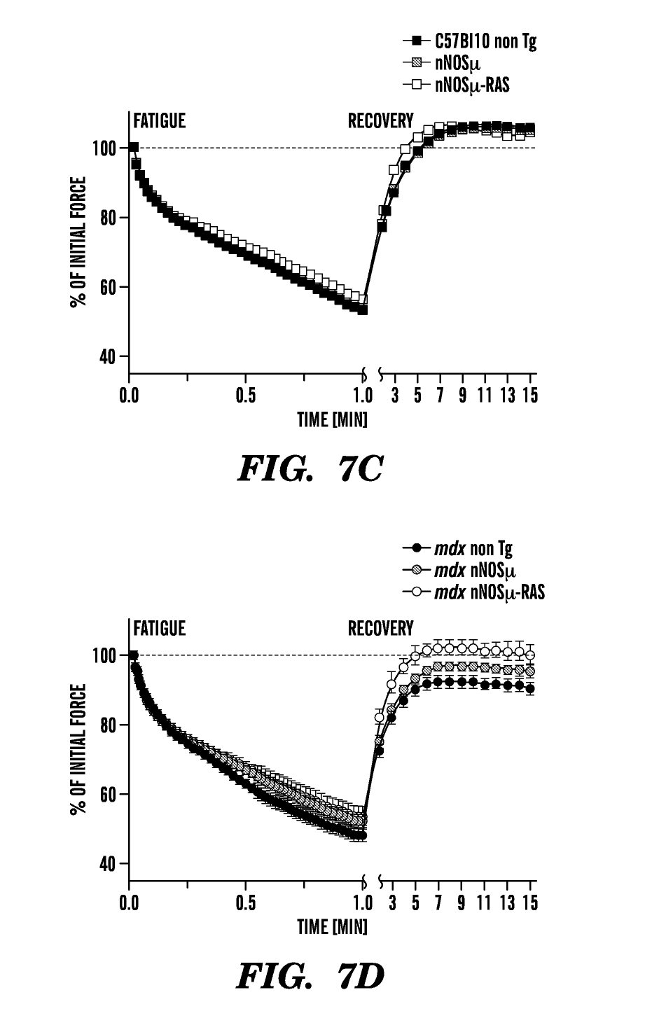

We evaluated the diaphragm muscle performance as described previously (Whitehead et al., 2008) using small (2-4 mm wide) strips of tissue, trying to maintain intact fibers that go from the rib bone to the central tendon of the diaphragm. After determination of L₀, the SpF was measured at frequencies of 10, 30, 50, 80, 100 and 120 Hz, and SpF vs Frequency was plotted comparing control and transgenic lines. Expressions of nNOSμ or nNOSμ-RAS have not effect on SpF in C57Bl10 or mdx mice (FIG. 7A). We also measured the twitch specific force, the force evoked for a single stimulus. Again, expression of nNOSμ or nNOSμ-RAS does not affect the twitch SpF in C57Bl10 or mdx diaphragm (FIG. 7B).

After determining the specific force, we performed a fatigue protocol. The diaphragm strip was subjected to a tetanic stimulation (120 Hz) every 1 second for 1 minute. This first step decreases muscle force after each contraction. Then, in a second step, a tetanic stimulation was performed every 1 minute for at least 10 minutes, to evaluate force recovery. When expressed in C57Bl10, nNOSμ or nNOSμ-RAS do not change the fatigue or the recovery phase of the protocol (FIG. 7C). This result indicates that over expression of nNOSμ has no effect on fatigue susceptibility in non-dystrophic diaphragm. Same experiments were performed on diaphragm of nNOSμ transgenic mice in the mdx background. We found that transgenic mice expressing nNOSμ in the cytosol show slightly more diaphragm fatigue susceptibility than the mdx negative littermates, which can be seen in both fatigue and recovery steps of the protocol. On the other hand, diaphragm from mdx mice expressing nNOSμ-RAS show similar force decay than control mdx mice, but they recover faster and reach the 100% of the initial force, a value that is never seen in a regular mdx mouse (FIG. 7D). This result indicates that sarcolemmal localized nNOSμ reduces fatigability.

### In Situ Evaluation of Tibialis Anterior Performance

We performed in situ physiology analysis of TA muscle (Percival et al., 2008), allowing for measurement of contractile properties without removing the muscle from its natural environment, maintaining normal vasoregulation and innervation. Briefly, mice were anesthetized with avertin, the TA muscle was surgically isolated and the tendon attached with surgical silk to the force transducer arm, while 2 electrodes were placed on the sciatic nerve. The mice were kept asleep with small doses of avertin every 15-20 minutes, while resting on a heated (37° C.) platform. We performed Length vs. Tension curve to determine L₀. Then, the SpF was measured at frequencies of 10, 30, 50, 80, 100, 150 and 200 Hz, and SpF vs Frequency was plotted comparing control and transgenic lines. SpF was calculated from both left and right hind limbs of each mouse. One hind limb was used to perform the fatigue resistance protocol, while the second hind limb was used to perform ECC-induced injury (explained below), alternating right and left extremities between experiments.

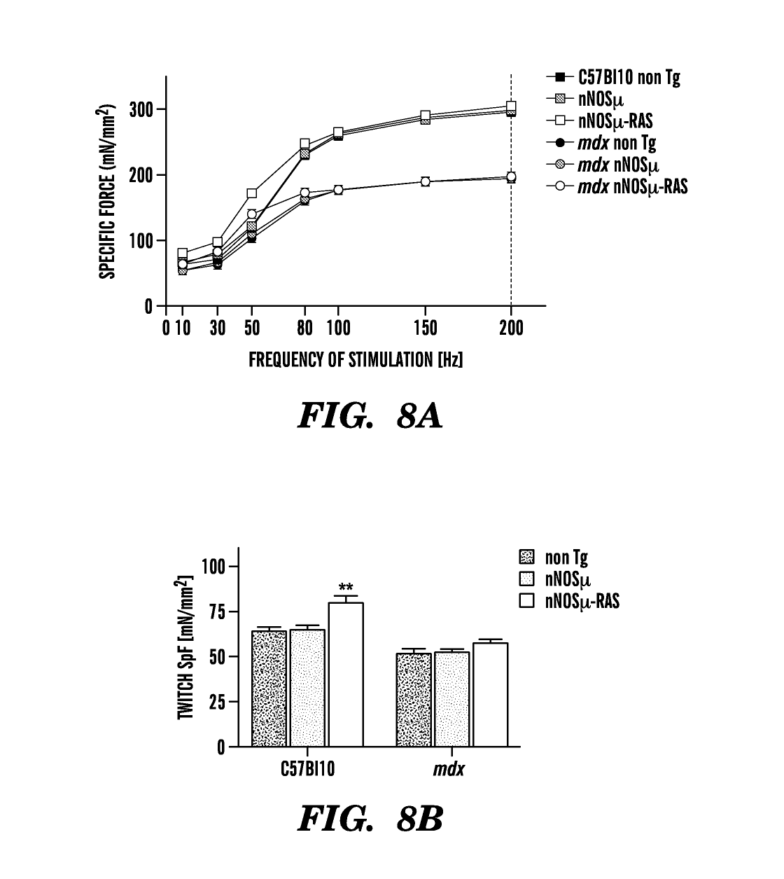

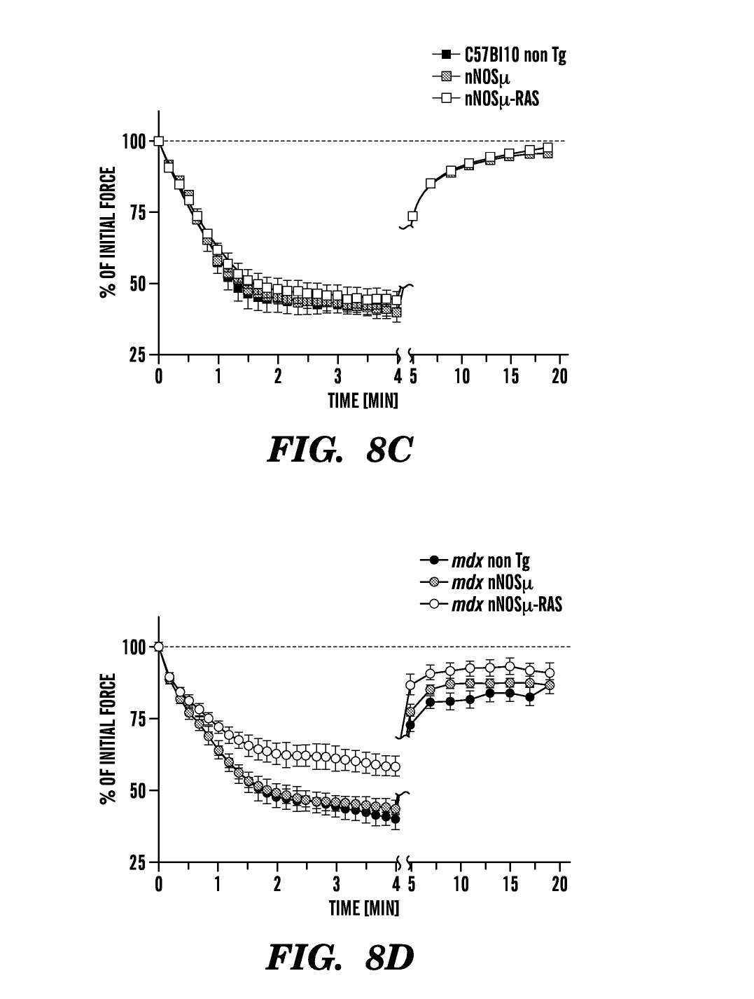

Expressions of nNOSμ have no effect on TA SpF in C57Bl10 or mdx mice (FIG. 8A). Similarly, TA SpF is not significantly different when nNOSμ-RAS is expressed. However, slightly higher forces in both C57Bl10 and mdx background are observed when stimulated at low frequencies (FIG. 8A). Then, we measured the TA twitch specific force (FIG. 8B). Expression of nNOSμ does not affect the twitch SpF in both C57Bl10 and mdx TA. However, nNOSμ-RAS expression tends to increase twitch SpF in normal and dystrophic TA, although only in C57Bl10 is the effect statistically significant. This is consistent with the observation of slightly higher forces when stimulated at low frequencies.

For fatigue experiments, TA muscle was subjected to tetanic stimulation (200 Hz) every 2 seconds during 4 minutes (120 contractions) to induce fatigue. After the last contraction, a tetanic stimulation every 2 minutes for at least 15 minutes was performed to evaluate TA muscle recovery from fatigue. We observed that transgenic mice expressing nNOSμ in the cytosol or sarcolemmal localized nNOSμ show the same TA fatigue and recovery pattern as control C57Bl10 mice (FIG. 8C). When analyzing dystrophic TA from mice in the mdx background, nNOSμ transgenic mice do not show significant differences with regular mdx mice; however, nNOSμ-RAS expression leads to significantly less fatigue in mdx mouse TA muscle (FIG. 8D).

Twitch Kinetics in Dystrophic Muscle

We evaluated a possible mechanism for the fatigue resistance in skeletal muscle from mdx nNOSμ-RAS expressing mice. Less susceptibility to fatigue could be due to increased numbers of slow twitch fibers and fewer fast twitch fibers. Different kinds of muscle fibers have different properties. Fast twitch fibers utilize glycolytic metabolism and thus fatigue more easily than slow twitch myofibers. For example, fibers expressing type I myosin heavy chain (MyHC) are the most fatigue resistant, while those expressing type IIB MyHC are the least fatigue resistant (Larsson and Phillips, 1998). Muscle fatigue susceptibility can be modulated by changes in the ratio of fatigue-resistant to fatigue susceptible muscle fibers (Percival et al., 2010; Selsby et al., 2012; Summermatter et al., 2012). A higher proportion of slow twitch fibers would increase the time for the muscle to reach peak twitch tension (TPT) and/or the relaxation phase, measured as the time to reach half of the relaxation (HRT). Analyzing Twitch kinetics parameters, we observed that TPT and HRT were invariable in Diaphragm from nNOSμ expressing mice when comparing with regular mdx (Table 2). In contrast, we observed increases in both TPT and HRT in mdx expressing nNOSμ-RAS. The same observation was made for Twitch kinetics in TA muscle. This result suggests a possible change in the fiber composition of the DIA and TA muscle as a mechanism for the decreased fatigability of nNOSμ-deficient muscle.

### In Situ Evaluation of Eccentric Contraction-Induced Damage in TA Muscle.

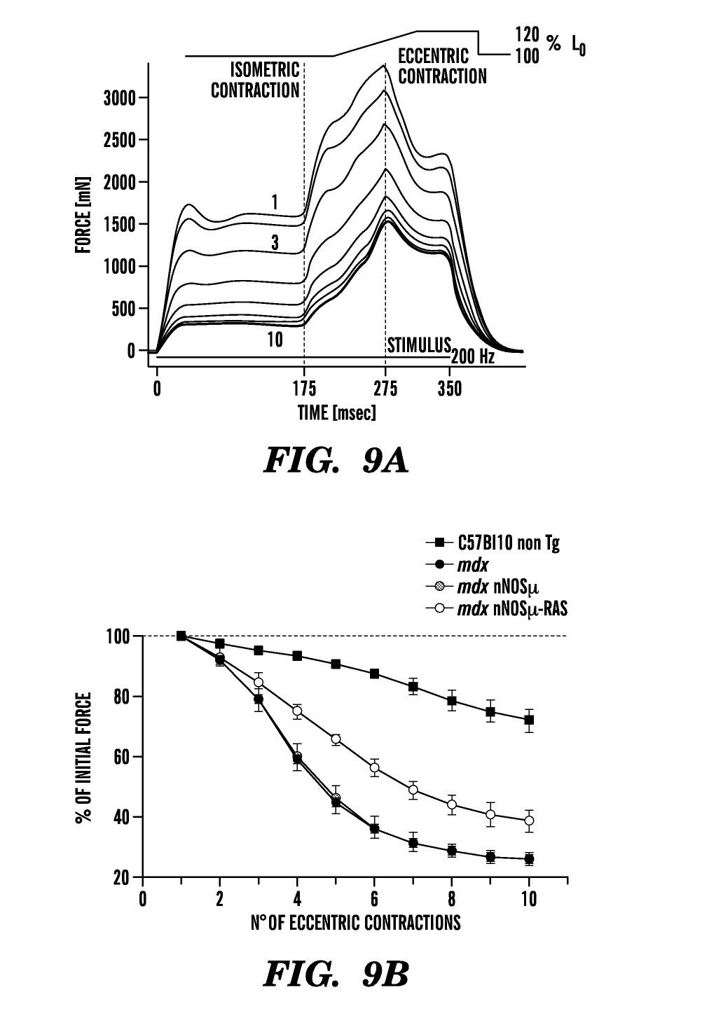

We used the same in situ methodology used for TA Fatigue susceptibility experiments to evaluate Eccentric Contraction-induced damage in mdx mice. Eccentric contraction (ECC) is contraction during elongation of the muscle. It allows muscle fibers to reach higher levels of tension, above levels reached in isometric (no length change) contraction (Blaauw et al., 2010). This can occur when an external force exceeds that produced by the muscle and the muscle lengthens, producing negative work (LaStayo et al., 2003). When repetitive, ECC can lead to muscle damage, characterized by structural and ultrastructural alterations, decreased force and pain (LaStayo et al., 2003; Proske and Morgan, 2001). Dystrophic muscles are more susceptible to damage induced by ECC, and can be detected after a few repetitions. This has been demonstrated in dystrophin null murine model (mdx) as well in others like α₂-laminin and γ-sarcoglycan-deficient mice, models for human congenital muscular dystrophy and limb girdle muscular dystrophy (Blaauw et al., 2010). Thus, augmented ECC-induced damage is a common pathological dysfunction. We used a protocol that simulates ECC in TA muscle. We did not evaluate this parameter in Diaphragm, because this muscle does not experience ECC normally. Each contraction is the result of a tetanic stimulus (200 Hz) in which during the first stimulus, the muscle executes an isometric contraction. Then, the muscle is stretched to a new length that is 20% longer than L₀, at a velocity of 2 L₀ per second, and the new length is held until the end of the stimulus. The stretching of the muscle causes a decrease in the maximal isometric force of the following contraction. A round of 10 consecutive eccentric contractions was performed every 1 minute. (FIG. 9A). The percentage of change between consecutive contractions was then compared between different mice. After 10 ECC, wt C57Bl10 mice presents just a 25% percent of force reduction (FIG. 9B), which after a few minutes is almost totally recovered. On the other hand, mdx mice presents exacerbated ECC-induced damage, decreasing the force to a 44% after 5 ECC, and to around 25% at the end of the protocol. Interestingly, we observed a reduction in the damage caused by ECC in TA muscle from mdx expressing sarcolemmal localized NOSμ-RAS, when compared to regular mdx mice (FIG. 9B). In contrast, expression of nNOSμ showed no improvement.

### Evaluation of Muscle Pathological Markers

Although the principal aim of this work was characterize the physiological properties of nNOSμ transgenic mice, we also evaluated some typical pathological markers of dystrophic muscle presents in our mdx model. The main purpose was to identify mechanisms through which nNOSμ-RAS decreases muscle fatigability and ECC-induced damage in mdx mice.

### Centronucleation and Fiber Area Variation.

In fibers from normal muscle, nuclei are located at the periphery near the sarcolemma. Upon a damage, muscle experience necrosis and then regeneration. In the regeneration process, regenerating fibers have central nuclei, which after a few days move to the normal peripheral location. In the same context, regenerating fibers are smaller, achieving a normal size at the end of the regeneration process (Cabral et al., 2008; Karalaki et al., 2009). In DMD and several other myopathies, chronic damage leads to ongoing and probably incomplete cycles of degeneration/regeneration, and the percentage of central nuclei rises. However, no consensus exists about the true meaning of centronucleation and its changes. While some investigators associate increased centronucleation with higher muscle damage, it is also possible that centronucleation is a sign of regeneration and appearance of new fibers. If the damage of the muscle remains constant, increasing regeneration can help to restore muscle function.

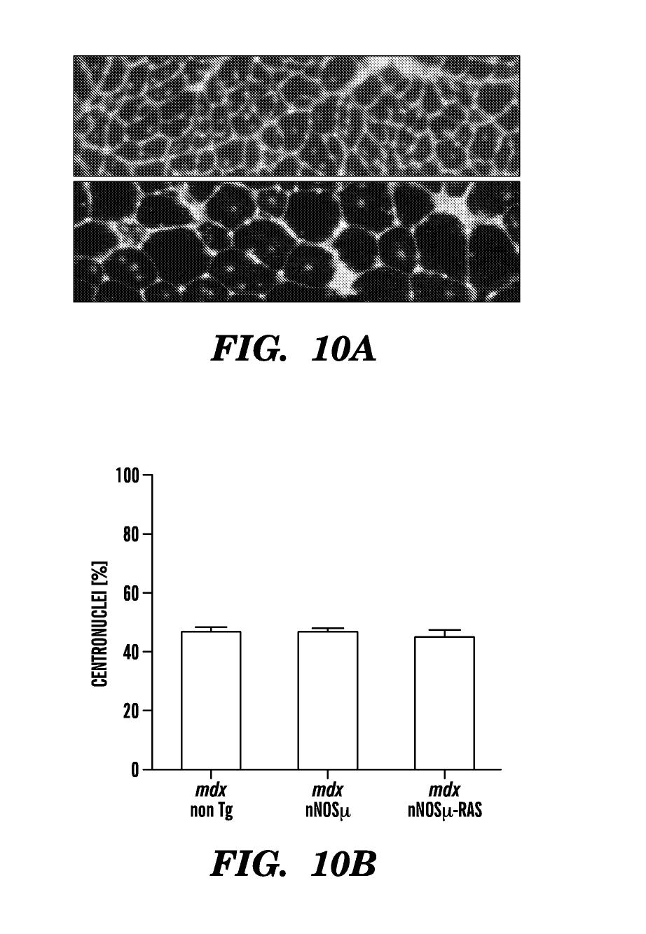

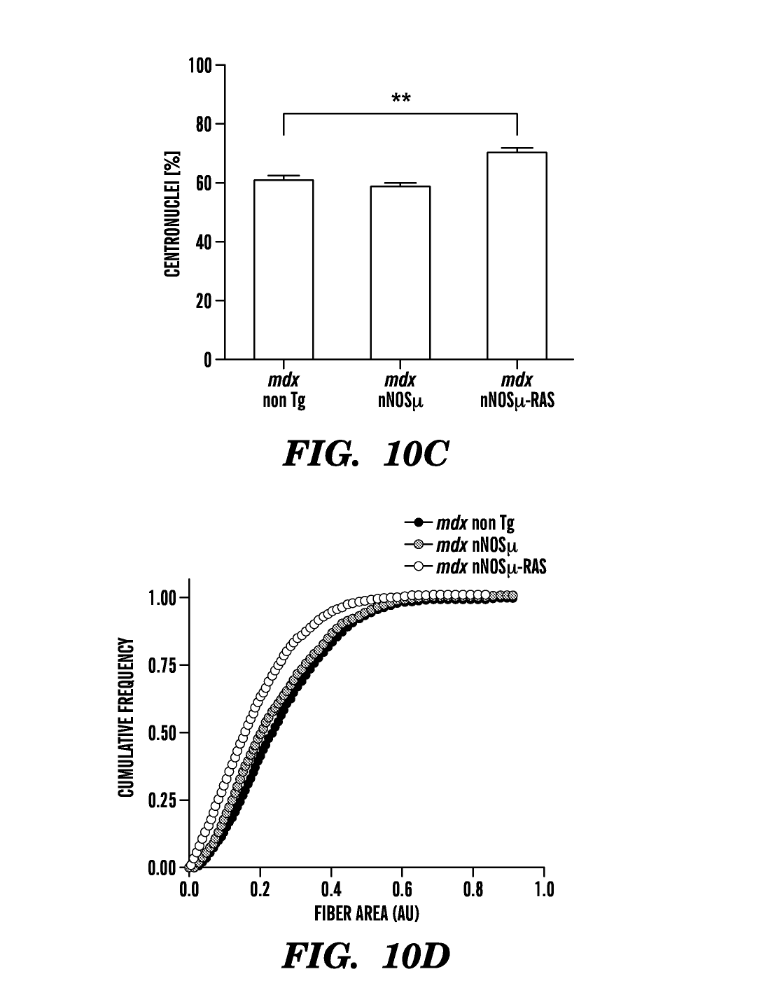

We evaluated the effect of cytosolic and sarcolemmal localized nNOSμ on muscle fiber regeneration through the determination of centronucleated in mdx and mdx transgenic mice. We used Wheat Germ Agglutinin (WGA)-488 and DAPI to label the sarcolemmal membrane and nuclei, respectively (FIG. 10A). This fluorescent staining allows a clear view and reliable quantification of centronucleai and fiber area, superior to classical H&E staining We evaluated centronucleation in Diaphragm and TA muscles from the fluorescence images obtained using Image J Cell counter plugin. We did not find differences in centronucleation in diaphragm from mdx nNOSμ and mdx nNOSμ-RAS transgenic mice when compared with regular mdx (FIG. 10B). However, we found a significant increase of centronuclei in TA muscle from sarcolemmal expressed nNOSμ compared to regular mdx and to mdx with cytosolic expression of nNOSμ (FIG. 10C).

Because regenerating fibers have a smaller caliber, we evaluated TA fiber size (cross-sectional area) distribution to determine the presence of smaller regenerating fibers. We measured 800-1000 fibers per muscle, distributed throughout the TA, to perform a cumulative frequency graph of fiber cross area. We found that TA fiber area distribution for cytosolic nNOSμ transgenic in the mdx background is very similar to regular mdx. In contrast, expression of sarcolemmal localized nNOSμ has a different distribution, showing a higher proportion of smaller fibers (FIG. 10D). These results together suggest that transgenic mice expressing sarcolemal localized nNOSμ have higher levels of regeneration in hind limb muscles. Former observations already have indicated that nNOS and NO may affect muscle differentiation and regeneration (Anderson, 2000; Lee et al., 1994; Wehling et al., 2001). However, our data conflict with those of Wheling et al, 2001, where the expression of cytosolic nNOSα increased regeneration. Our cytosolic nNOSμ transgenic mice do not have this difference. Without wishing to be bound or limited by theory, it is possible that the difference relates to the use of skeletal muscle (μ) nNOS instead of the brain (α) isoform in the studies described herein.

### Fibrotic Markers Evaluation

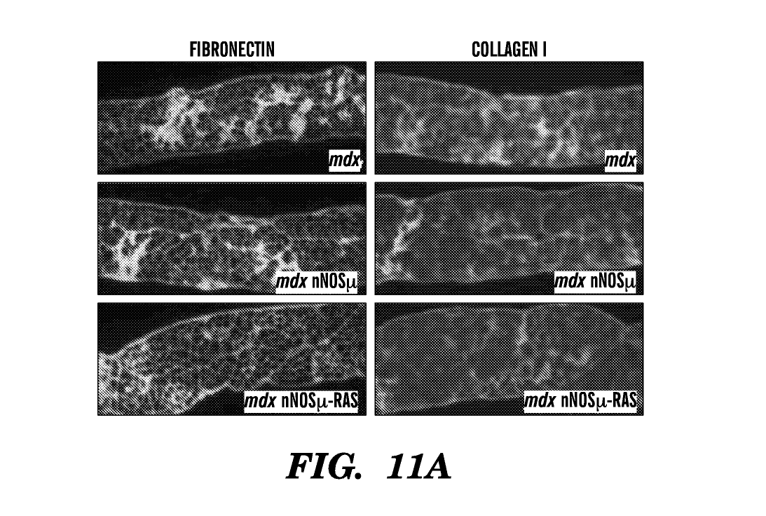

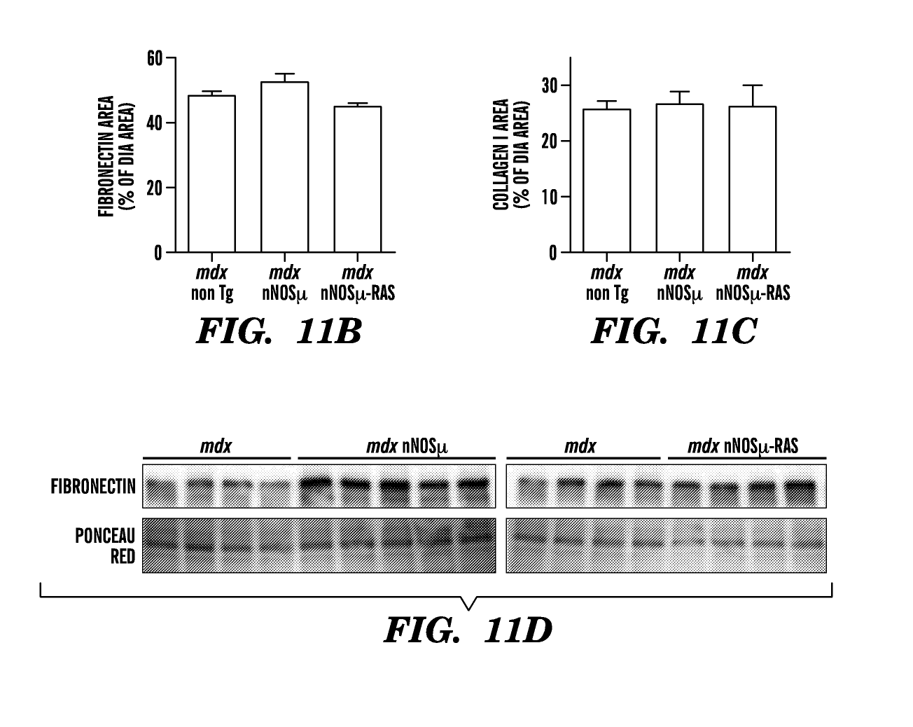

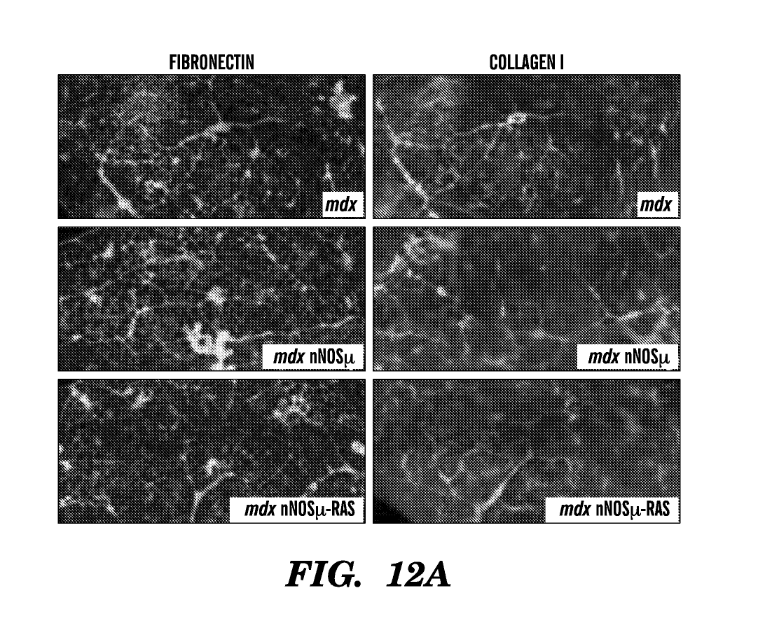

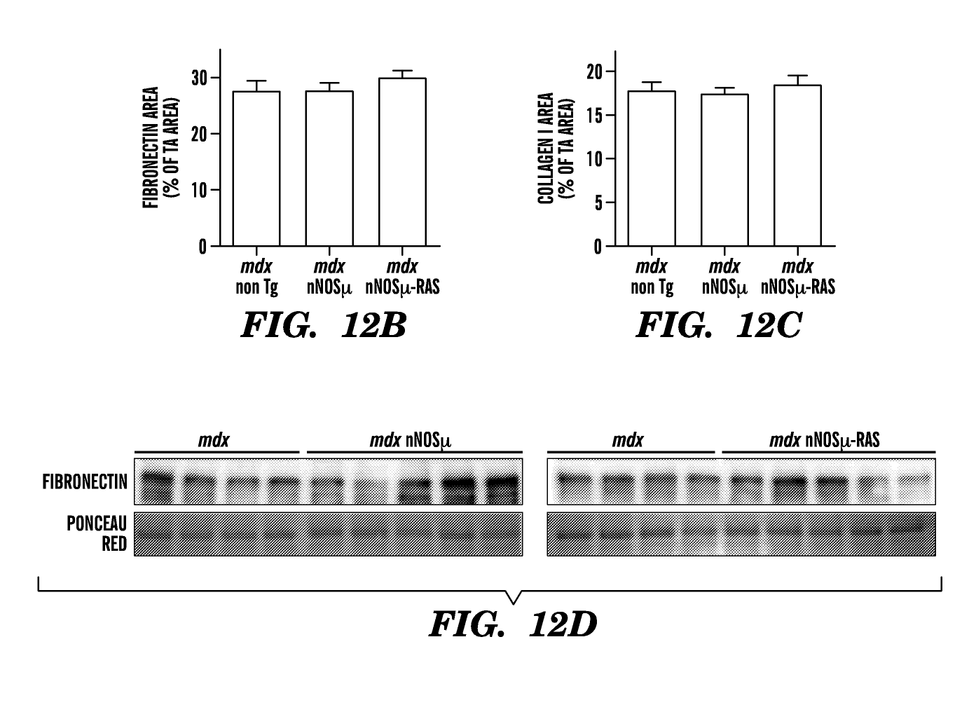

DMD and its mdx mouse model, as in other muscular dystrophies, are characterized by fibrosis. Fibrosis is an excessive accumulation of extracellular matrix (ECM) components such as Collagen and Fibronectin (Serrano et al., 2011; Serrano and Munoz-Canoves, 2010; Wynn, 2008) that replaces the functional tissue, decreasing muscle force and normal regeneration. Also, increased fibrosis generates a physical barrier that opposes neovascularization, necessary for normal muscle regeneration and function (Gargioli et al., 2008). Previous work had shown possible that NO signaling can have effects on fibrosis. Sildenafil is a selective inhibitor of phosphodiesterase (PDE) 5 that enhances the cGMP-dependent pathway of NO signaling. Administration of sildenafil to mdx mice via the drinking water followed by examination of skeletal muscle function, showed that chronic, long-term treatment significantly reduced mdx diaphragm muscle weakness and reduced mdx diaphragm fibrosis (Percival et al., 2012). We determined if fibrosis markers were affected by nNOSμ or nNOSμ-RAS expression. We used 10-μm-thick frozen sections to perform immunofluorescence with antibodies against Fibronectin and Colagen I and analyzed the accumulation of both ECM components. We used whole TA and hemidiaphragm to analyze and quantify the area occupied by Fibronectin and Collagen I. When analyzing diaphragm, we found no differences in either Fibronectin and Collagen I between muscles from mdx and mdx expressing sarcolemmal localized nNOSμ. Muscle with cytosolic nNOSμ showed increased fibronectin accumulation compared to regular mdx, but the difference is not statistically significant (FIGS. 11A-11C). The same trend was observed in western blot analysis for fibronectin (FIG. 11D). When analyzing TA, we found no differences in fibrotic markers between mdx and both sarcolemmal localized nNOSμ and cytosolic localized nNOSμ (FIGS. 12A-12D). Our results indicate that expression of nNOSμ or nNOSμ-RAS has, at most, a small effect on fibrosis in mdx muscle, at least at the age tested. We used 2 month old mice for muscle physiology characterization, and muscle slides and homogenates were obtained from the same mice. Fibronectin and Collagen deposition increases with time in mdx, and it is possible that effects of transgenic expression of nNOSμ on fibrosis occurs only later in the pathology of the mdx mice.

nNOSμ Ability to Direct DGC Members to the Sarcolemma

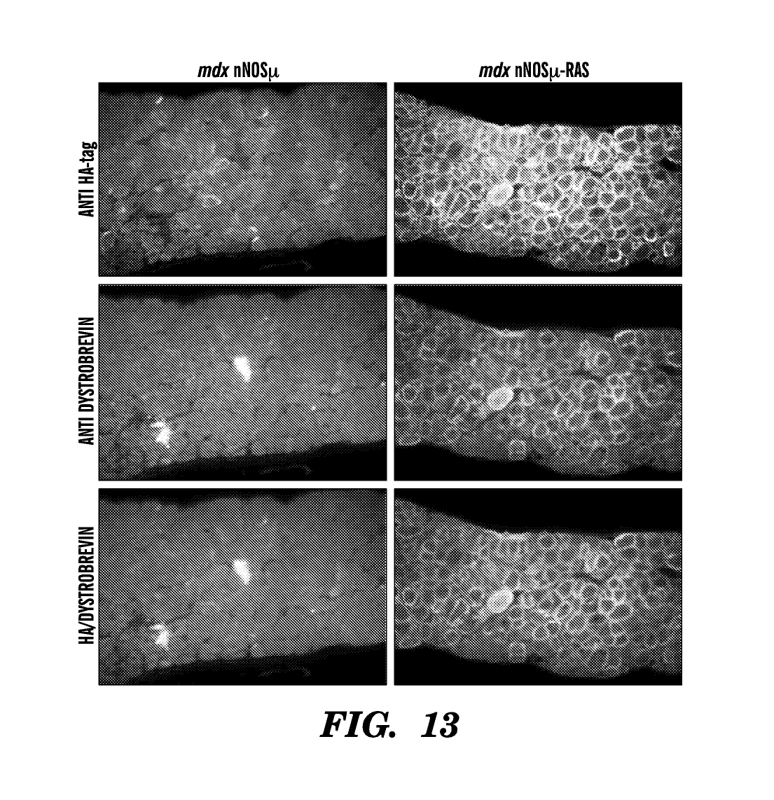

DMD and is caused by mutations in the dystrophin gene, which encodes an actin binding protein that links the actin cytoskeleton with the extracellular matrix, forming the dystrophin-associated glycoprotein complex (DGC) (FIG. 2). The dystrophin deficiency leads to loss of other DGC members, including nNOSμ (Ibraghimov-Beskrovnaya et al., 1992; Waite et al., 2009). nNOSμ interacts with α-syntrophin through its PDZ domain, connecting nNOSμ to dystrophin and dystrobrevin (see, for example, FIG. 2). Because of one of the proposed roles of DGC is maintainence of membrane integrity, we determined if nNOSμ-RAS transgenic protein is able to restore the sarcolemmal localization of other members of the complex. In sarcolemmal localized nNOSμ, we detected α-dystrobrevin colocalization with nNOSμ-RAS on the sarcolemma (FIG. 13), localization driven most probably through interaction with α-syntrophin. Because of technical limitations, we could not evaluate syntrophin localization. We did not observe sarcolemmal localization of nNOSμ or dystrobrevin on transgenic mice expressing nNOSμ.

Abnormally high levels of NO, especially at subcellular locations where the levels are usually low, can have major detrimental effects. We reasoned that targeting of nNOSμ to the sarcolemma, its normal location, is an essential requirement for improving the dystrophic phenotype. Production of NO at the sarcolemma would increase the likelihood that the appropriate physiological targets would be hit, while at the same time reducing NO mediated damage caused by abnormally high levels of cytosolic nNOSμ. As described above, in this work we took a novel approach to achieve a normal distribution of nNOSμ in skeletal muscle. First, for the first time, we produced transgenic mice expressing nNOSμ, the skeletal muscle isoform. Previous publications have used the brain nNOSα isoform, which lack 34 amino acids characteristic of the μ-domain, whose function is incompletely elucidated. Second, we introduced a modification adding the RAS palmitoylation signal sequence at the C-terminus, resulting in palmitoylation of nNOSμ, a modification that drives the protein to the sarcolemma independent of dystrophin and α-syntrophin presence. We obtained transgenic lines for both nNOSμ and nNOSμ-RAS transgenes.

In wild type mice, nNOSμ can normally localize to the sarcolemma because of the intact DGC complex. It is known that nNOSμ expression and activity increases with age in mice and rats at least until 1 year of age (Chang et al., 1996; Stamler and Meissner, 2001). Furthermore, it is also known that nNOSμ expression is increased by exercise in both rodents and humans, which has been related to a role of nNOSμ in adaptation of skeletal muscle to endurance exercise (Percival, 2011). Modifying nNOSμ expression with the ectopic transgene could mimic the effects of exercise training in wild type muscles. In this context, we expected to observe some increments on muscle force or fatigue resistance when increasing nNOSμ (or nNOSμ-RAS), speeding processes that improve muscle performance. However, we did not observe these effects.

We focused on functional studies to evaluate in dystrophic skeletal muscle the impact of sarcolemmal nNOSμ-RAS and cytosolic nNOSμ. Perhaps the most important result was the reduction in the damage caused by eccentric contractions in the mdx nNOSμ-RAS TA muscle, a common pathological marker of DMD and the mdx model. Earlier studies have shown that nNOSμ absence has a major impact in increasing muscle fatigue susceptibility in normal, non-dystrophic muscle (Percival et al., 2008; Percival et al., 2010). Using an in situ protocol, we have now examined the effect of sarcolemmal targeted nNOSμ on muscle fatigue. Fatigue in the mdx TA muscle was markedly reduced by RAS-nNOSμ on the sarcolemma. Fatigue susceptibility was also reduced in diaphragm. We did not observe significant changes in fibrosis, suggesting that the mechanism of action is not through the diminution of fibrotic markers at the age tested (2 month old).

We observed a slower twitch kinetic in muscles from nNOSμ-RAS expressing mice. These results indicate that fiber type shift can be one of the mechanisms, but certainly not the only one, in decreasing fatigue susceptibility in mdx mice nNOSμ-RAS. Sarcolemmal localized nNOSμ is essential in opposing vasoconstriction during exercise through NO production and maintaining blood flow and oxygen availability (Thomas et al., 1998; Thomas et al., 2003). In DMD, DGC loss includes delocalization and decreased expression of nNOSμ, while nNOSμ loss from the sarcolemma is also observed in models of denervation and ALS (Brenman et al., 1995; Chao et al., 1996; Crosbie et al., 2002; Meinen et al., 2012; Percival, 2011; Suzuki et al., 2010; Suzuki et al., 2007). This common phenomenon, added to the reduced vascularization in muscular dystrophy (Gargioli et al., 2008), can trigger ischemic foci and a detrimental hypoxic environment. Restoring nNOSμ to the sarcolemma would help to maintain oxygen availability during muscle contraction, improving energy production for muscle contraction, reducing fatigue, and preventing necrotic foci because of hypoxia.

We obtained evidence indicating that expression of nNOSμ localized in the sarcolemma can promote regeneration in dystrophic TA muscle. Previously, it has been reported that nNOSμ inhibition or its absence (mdx or nNOS knockout) negatively affect satellite cell activation, including morphological hypertrophy and decreased adhesion in the fiber-lamina complex (Anderson, 2000). In addition, NO donors can increase myogenesis and muscle repair (De Palma and Clementi, 2012; Stamler and Meissner, 2001). These reports do not consider localization of NO production, and literature indicates that both cytosolic and sarcolemmal nNOSμ are important in muscle function. Our results indicate that in mdx mice expression of sarcolemmal but not cytosolic nNOSμ is able to induce regeneration. Actions of NO on skeletal muscle mentioned above, such as vasodilation and reduction of ischemia induced by nNOSμ displacement, increase in glucose uptake and its angiogenic effects (Stamler and Meissner, 2001), are all facilitated by NO production at the sarcolemma. Our novel mode of sarcolemmal localization may contribute to muscle repair and to reduced ECC-induced damage and fatigue susceptibility (Schgoer et al., 2009; Ziche et al., 1994).

The role of cytoplasmic nNOSμ is a controversial issue. In the nNOSμ/dystrophin double knockout mouse, the loss of nNOSμ results in an increase in the strength of the EDL and TA muscles (Li et al., 2011). Thus, cytosolic nNOSμ may have a toxic function in dystrophic muscle, a function already suggested for non-dystrophic muscles. α1-Syntrophin-null muscles show displacement of nNOSμ from the sarcolemma and do not regenerate normally (Hosaka et al., 2002), while in tail-suspension, denervation and Amyotrophic Lateral Sclerosis (ALS) models, nNOSμ is mislocalized and induces muscle atrophy through the activation of FoxO3a and muscle-specific E3 ubiquitin ligases MuRF-1 and atrogin-1/MAFbx (Suzuki et al., 2010; Suzuki et al., 2007). Taking this into account, and while not wishing to be bound or limited by theory, improvement of muscle performance in nNOSμ-RAS expressing mice can also be due, at least in part, to a reduction of the toxic effects of cytosolic endogenous nNOSμ. nNOS forms active dimers (Zhou and Zhu, 2009), therefore monomeric nNOSμ with the RAS-tag could associate with endogenous nNOSμ and recruit it to the sarcolemma in the absence of dystrophin. Here, an additive action between restoration of normal function and diminution of toxic, cytosolic function could account for muscle performance improvement in nNOSμ-RAS transgenic mice. Nevertheless, we did not observe a generalized worsening of muscle function in transgenic mice expressing cytosolic nNOSμ. While no effects were observed on TA muscles, a slight increase in fatigue susceptibility and fibronectin accumulation was observed in Diaphragm from nNOSμ transgenic mice.

Finally, we observed recruitment of the DGC-member Dystrobrevin to the sarcolemma of nNOSμ-RAS expressing mice. DGC proteins are important for establishing the physical connection between the extracellular matrix and the cytoskeleton and play a role in transmitting force related to muscle contraction (Allikian and McNally, 2007; Waite et al., 2009). While not wishing to be bound or limited by theory, partial restoration of DGC members may help to maintain membrane integrity and interaction with different signaling pathways that are lost in the absence of dystrophin, resulting in a milder phenotype.

TABLE 1
Primers used for cloning, sequencing
and modify nNOSμ Cdna (SEQ ID NOs: 30-51,
in order of appearance)
Cloning primers

| | |
|---|---|
| nnos5b | 5′-ccg gag tag ctc agg ttc ctg tg-3′ |
| nnos3b | 5′-gtg ggc act cag ggc cac cac-3′ |
| nos5aNot | 5′-cct taa tta aca gcc acc cca tat |
| | ccc atg-3′ |
| nos3aPac | 5′-cct taa tta aca gcc acc cca tat |
| | ccc atg-3′ |
| mup1 | 5′-gtc ttc cac cag gag atg-3′ |
| mup2 | 5′-aaa ggc aca gaa gtg ggg gta-3′ |
| HA1 | 5′-cac aga tga ggt ttt cag ctc cta |
| | ccc ata tga cgt tcc tga cta cgc atc |
| | cct tta att aat taa gg-3′ |
| HA2 | 5′-gcc tta att aat taa agg gat gcg |
| | tag tca gg-3′ |
| HARAS1 | 5′-gag gtt ttc agc tcc tac cca tat |
| | gac gtt c-3′ |
| HARAS2 | 5′-cgg atc gta tta att aag gcg ccg |
| | cgg-3′ |

Sequencing primers

| | |
|---|---|
| Forward 1 | 5′-gac cag cca tta gca gta gac-3′ |
| Forward 2 | 5′-ttc tcg acc aat act act c-3′ |
| Forward 3 | 5′-gtg tcc aac atg ctg ctg g-3′ |
| Forward 4 | 5′-ctg tgc gag atc ttc aag c -3′ |
| Forward 5 | 5′-gaa gct cca gag ctg acc-3′ |
| Forward 6 | 5′-ctc aca cag ctg tcg ctg-3′ |
| Reverse 1 | 5′-aat ggc cac acc aft agc ctg-3′ |
| Reverse 2 | 5′-tgt cct tga gct ggt agg tg-3′ |
| Reverse 3 | 5′-agt aat cac gaa cgc caa tc-3′ |
| Reverse 4 | 5′-ttg cca aag gtg ctg gtg ac-3′ |
| Reverse 5 | 5′-gaa gat ggt cga tcg gct g-3′ |
| Reverse 6 | 5′-gta tgg tag gac acg atg gc-3′ |

TABLE 2
Twitch kinetics in Dystrophic muscle

| | | mdx non Tg | mdx nNOSμ | mdx nNOSμ-RAS |
|---|---|---|---|---|
| DIA | TPT | 37.14 ± 1.12 | 37.7 ± 0.88 | 47.46 ± 0.85 *** |
| | HRT | 47.97 ± 0.53 | 37.42 ± 1.34 | 39.5 ± 1.64 *** |
| TA | TPT | 20.51 ± 0.47 | 20.17 ± 0.29 | 22.81 ± 0.49 ** |
| | HRT | 10.48 ± 0.35 | 10.25 ± 0.31 | 14.54 ± 0.8 ** |

### REFERENCES

Allikian, M. J., and E. M. McNally. 2007. Processing and assembly of the dystrophin glycoprotein complex. Traffic. 8:177-183.
Anderson, J. E. 2000. A role for nitric oxide in muscle repair: nitric oxide-mediated activation of muscle satellite cells. Mol Biol Cell. 11:1859-1874.
Bender, A. T., and J. A. Beavo. 2006. Cyclic nucleotide phosphodiesterases: molecular regulation to clinical use. Pharmacol Rev. 58:488-520.
Blaauw, B., L. Agatea, L. Toniolo, M. Canato, M. Quarta, K. A. Dyar, D. Danieli-Betto, R. Betto, S. Schiaffino, and C. Reggiani. 2010. Eccentric contractions lead to myofibrillar dysfunction in muscular dystrophy. J Appl Physiol. 108:105-111.
Brenman, J. E., D. S. Chao, S. H. Gee, A. W. McGee, S. E. Craven, D. R. Santillano, Z. Wu, F. Huang, H. Xia, M. F. Peters, S. C. Froehner, and D. S. Bredt. 1996. Interaction of nitric oxide synthase with the postsynaptic density protein PSD-95 and alpha1-syntrophin mediated by PDZ domains. Cell. 84:757-767.
Brenman, J. E., D. S. Chao, H. Xia, K. Aldape, and D. S. Bredt. 1995. Nitric oxide synthase complexed with dystrophin and absent from skeletal muscle sarcolemma in Duchenne muscular dystrophy. Cell. 82:743-752.
Cabral, A. J. V., V. Machado, R. Farinha, and A. Cabrita. 2008. Skeletal muscle regeneration: a brief review
António J. V. Cabral1, Vanessa Machado1, Rodrigo Farinha1, António Cabrita1. Experimental Pathology and Health Sciences

2008; 2 (2): 9-17. 2:9.
Chang, W. J., S. T. Iannaccone, K. S. Lau, B. S. Masters, T. J. McCabe, K. McMillan, R. C. Padre, M. J. Spencer, J. G. Tidball, and J. T. Stull. 1996. Neuronal nitric oxide synthase and dystrophin-deficient muscular dystrophy. Proc Natl Acad Sci USA. 93:9142-9147.
Chao, D. S., J. R. Gorospe, J. E. Brenman, J. A. Rafael, M. F. Peters, S. C. Froehner, E. P. Hoffman, J. S. Chamberlain, and D. S. Bredt. 1996. Selective loss of sarcolemmal nitric oxide synthase in Becker muscular dystrophy. J Exp Med. 184:609-618.
Craven, K. B., and W. N. Zagotta. 2006. CNG and HCN channels: two peas, one pod. Annu Rev Physiol. 68:375-401.
Crawford, G. E., J. A. Faulkner, R. H. Crosbie, K. P. Campbell, S. C. Froehner, and J. S. Chamberlain. 2000. Assembly of the dystrophin-associated protein complex does not require the dystrophin COOH-terminal domain. J Cell Biol. 150:1399-1410.
Crosbie, R. H., R. Barresi, and K. P. Campbell. 2002. Loss of sarcolemma nNOS in sarcoglycan-deficient muscle. Faseb J. 16:1786-1791.
De Palma, C., and E. Clementi. 2012. Nitric oxide in myogenesis and therapeutic muscle repair. Mol Neurobiol. 46:682-692.
Durbeej, M., and K. P. Campbell. 2002. Muscular dystrophies involving the dystrophin-glycoprotein complex: an overview of current mouse models. Curr Opin Genet Dev. 12:349-361.
Ehmsen, J., E. Poon, and K. Davies. 2002. The dystrophin-associated protein complex. J Cell Sci. 115:2801-2803.
Eliasson, M. J., S. Blackshaw, M. J. Schell, and S. H. Snyder. 1997. Neuronal nitric oxide synthase alternatively spliced forms: prominent functional localizations in the brain. Proc Natl Acad Sci USA. 94:3396-3401.
Eu, J. P., J. M. Hare, D. T. Hess, M. Skaf, J. Sun, I. Cardenas-Navina, Q. A. Sun, M. Dewhirst, G. Meissner, and J. S. Stamler. 2003. Concerted regulation of skeletal muscle contractility by oxygen tension and endogenous nitric oxide. Proc Natl Acad Sci USA. 100:15229-15234.
Eu, J. P., J. Sun, L. Xu, J. S. Stamler, and G. Meissner. 2000. The skeletal muscle calcium release channel: coupled O2 sensor and NO signaling functions. Cell. 102:499-509.
Gargioli, C., M. Coletta, F. De Grandis, S. M. Cannata, and G. Cossu. 2008. PlGF-MMP-9-expressing cells restore microcirculation and efficacy of cell therapy in aged dystrophic muscle. Nat Med. 14:973-978.
Gregorevic, P., J. M. Allen, E. Minami, M. J. Blankinship, M. Haraguchi, L. Meuse, E. Finn, M. E. Adams, S. C. Froehner, C. E. Murry, and J. S. Chamberlain. 2006. rAAV6-microdystrophin preserves muscle function and extends lifespan in severely dystrophic mice. Nat Med. 12:787-789.
Gyurko, R., S. Leupen, and P. L. Huang. 2002. Deletion of exon 6 of the neuronal nitric oxide synthase gene in mice results in hypogonadism and infertility. Endocrinology. 143:2767-2774.
Hack, A. A., C. T. Ly, F. Jiang, C. J. Clendenin, K. S. Sigrist, R. L. Wollmann, and E. M. McNally. 1998. Gamma-sarcoglycan deficiency leads to muscle membrane defects and apoptosis independent of dystrophin. J Cell Biol. 142:1279-1287.
Hakim, C. H., D. Li, and D. Duan. 2011. Monitoring murine skeletal muscle function for muscle gene therapy. Methods Mol Biol. 709:75-89.
Hofmann, F., D. Bernhard, R. Lukowski, and P. Weinmeister. 2009. cGMP regulated protein kinases (cGK). Handb Exp Pharmacol:137-162.
Hosaka, Y., T. Yokota, Y. Miyagoe-Suzuki, K. Yuasa, M. Imamura, R. Matsuda, T. Ikemoto, S. Kameya, and S. Takeda. 2002. Alpha1-syntrophin-deficient skeletal muscle exhibits hypertrophy and aberrant formation of neuromuscular junctions during regeneration. J Cell Biol. 158:1097-1107.
Ibraghimov-Beskrovnaya, O., J. M. Ervasti, C. J. Leveille, C. A. Slaughter, S. W. Sernett, and K. P. Campbell. 1992. Primary structure of dystrophin-associated glycoproteins linking dystrophin to the extracellular matrix. Nature. 355:696-702.
Judge, L. M., and J. S. Chamberlain. 2005. Gene therapy for Duchenne muscular dystrophy: AAV leads the way. Acta Myol. 24:184-193.
Karalaki, M., S. Fili, A. Philippou, and M. Koutsilieris. 2009. Muscle regeneration: cellular and molecular events. In Vivo. 23:779-796.
Kobayashi, Y. M., E. P. Rader, R. W. Crawford, N. K. Iyengar, D. R. Thedens, J. A. Faulkner, S. V. Parikh, R. M. Weiss, J. S. Chamberlain, S. A. Moore, and K. P. Campbell. 2008. Sarcolemma-localized nNOS is required to maintain activity after mild exercise. Nature. 456:511-515.
Larsson, B., and S. C. Phillips. 1998. Isolation and characterization of a novel, human neuronal nitric oxide synthase cDNA. Biochem Biophys Res Commun. 251:898-902.
LaStayo, P. C., J. M. Woolf, M. D. Lewek, L. Snyder-Mackler, T. Reich, and S. L. Lindstedt. 2003. Eccentric muscle contractions: their contribution to injury, prevention, rehabilitation, and sport. J Orthop Sports Phys Ther. 33:557-571.
Lee, K. H., M. Y. Bask, K. Y. Moon, W. K. Song, C. H. Chung, D. B. Ha, and M. S. Kang. 1994. Nitric oxide as a messenger molecule for myoblast fusion. J Biol Chem. 269:14371-14374.
Li, D., Y. Yue, Y. Lai, C. H. Hakim, and D. Duan. 2011. Nitrosative stress elicited by nNOSmicro delocalization inhibits muscle force in dystrophin-null mice. J Pathol. 223:88-98.
Meinen, S., S. Lin, M. A. Ruegg, and A. R. Punga. 2012. Fatigue and muscle atrophy in a mouse model of myasthenia gravis is paralleled by loss of sarcolemmal nNOS. PLoS One. 7:e44148.
Miyagoe-Suzuki, Y., and S. I. Takeda. 2001. Association of neuronal nitric oxide synthase (nNOS) with alpha1-syntrophin at the sarcolemma. Microsc Res Tech. 55:164-170.
Percival, J. M. 2011. nNOS regulation of skeletal muscle fatigue and exercise performance. Biophysical Reviews. 3:9.
Percival, J. M., K. N. Anderson, P. Gregorevic, J. S. Chamberlain, and S. C. Froehner. 2008. Functional deficits in nNOSmu-deficient skeletal muscle: myopathy in nNOS knockout mice. PLoS One. 3:e3387.
Percival, J. M., K. N. Anderson, P. Huang, M. E. Adams, and S. C. Froehner. 2010. Golgi and sarcolemmal neuronal NOS differentially regulate contraction-induced fatigue and vasoconstriction in exercising mouse skeletal muscle. J Clin Invest. 120:816-826.
Percival, J. M., N. P. Whitehead, M. E. Adams, C. M. Adamo, J. A. Beavo, and S. C. Froehner. 2012. Sildenafil reduces respiratory muscle weakness and fibrosis in the mdx mouse model of Duchenne muscular dystrophy. J Pathol. 228:77-87.
Proske, U., and D. L. Morgan. 2001. Muscle damage from eccentric exercise: mechanism, mechanical signs, adaptation and clinical applications. J Physiol. 537:333-345.
Rando, T. A. 2001. The dystrophin-glycoprotein complex, cellular signaling, and the regulation of cell survival in the muscular dystrophies. Muscle Nerve. 24:1575-1594.
Ross, R. M., G. D. Wadley, M. G. Clark, S. Rattigan, and G. K. McConell. 2007. Local nitric oxide synthase inhibition reduces skeletal muscle glucose uptake but not capillary blood flow during in situ muscle contraction in rats. Diabetes. 56:2885-2892.
Sambrook, J., P. MacCallum, and D. Russell. 2001. Molecular Clonig: A Laboratory Manual.
Schgoer, W., M. Theurl, J. Jeschke, A. G. Beer, K. Albrecht, R. Gander, S. Rong, D. Vasiljevic, M. Egger, A. M. Wolf, S. Frauscher, B. Koller, I. Tancevski, J. R. Patsch, P. Schratzberger, H. Piza-Katzer, A. Ritsch, F. H. Bahlmann, R. Fischer-Colbrie, D. Wolf, and R. Kirchmair. 2009. Gene therapy with the angiogenic cytokine secretoneurin induces therapeutic angiogenesis by a nitric oxide-dependent mechanism. Circ Res. 105:994-1002.
Schillings, M. L., J. S. Kalkman, H. M. Janssen, B. G. van Engelen, G. Bleijenberg, and M. J. Zwarts. 2007. Experienced and physiological fatigue in neuromuscular disorders. Clin Neurophysiol. 118:292-300.
Selsby, J. T., K. J. Morine, K. Pendrak, E. R. Barton, and H. L. Sweeney. 2012. Rescue of dystrophic skeletal muscle by PGC-1alpha involves a fast to slow fiber type shift in the mdx mouse. PLoS One. 7:e30063.
Serrano, A. L., C. J. Mann, B. Vidal, E. Ardite, E. Perdiguero, and P. Munoz-Canoves. 2011. Cellular and molecular mechanisms regulating fibrosis in skeletal muscle repair and disease. Curr Top Dev Biol. 96:167-201.
Serrano, A. L., and P. Munoz-Canoves. 2010. Regulation and dysregulation of fibrosis in skeletal muscle. Exp Cell Res. 316:3050-3058.
Shiao, T., A. Fond, B. Deng, M. Wehling-Henricks, M. E. Adams, S. C. Froehner, and J. G. Tidball. 2004. Defects in neuromuscular junction structure in dystrophic muscle are corrected by expression of a NOS transgene in dystrophin-deficient muscles, but not in muscles lacking alpha- and beta1-syntrophins. Hum Mol Genet. 13:1873-1884.
Silvagno, F., H. Xia, and D. S. Bredt. 1996. Neuronal nitric-oxide synthase-mu, an alternatively spliced isoform expressed in differentiated skeletal muscle. J Biol Chem. 271:11204-11208.
Stamler, J. S., and G. Meissner. 2001. Physiology of nitric oxide in skeletal muscle. Physiol Rev. 81:209-237.
Stedman, H. H., H. L. Sweeney, J. B. Shrager, H. C. Maguire, R. A. Panettieri, B. Petrof, M. Narusawa, J. M. Leferovich, J. T. Sladky, and A. M. Kelly. 1991. The mdx mouse diaphragm reproduces the degenerative changes of Duchenne muscular dystrophy. Nature. 352:536-539.
Summermatter, S., R. Thurnheer, G. Santos, B. Mosca, O. Baum, S. Treves, H. Hoppeler, F. Zorzato, and C. Handschin. 2012. Remodeling of calcium handling in skeletal muscle through PGC-1alpha: impact on force, fatigability, and fiber type. Am J Physiol Cell Physiol. 302:C88-99.
Suzuki, N., H. Mizuno, H. Warita, S. Takeda, Y. Itoyama, and M. Aoki. 2010. Neuronal NOS is dislocated during muscle atrophy in amyotrophic lateral sclerosis. J Neurol Sci. 294:95-101.
Suzuki, N., N. Motohashi, A. Uezumi, S. Fukada, T. Yoshimura, Y. Itoyama, M. Aoki, Y. Miyagoe-Suzuki, and S. Takeda. 2007. NO production results in suspension-induced muscle atrophy through dislocation of neuronal NOS. J Clin Invest. 117:2468-2476.
Thomas, G. D., M. Sander, K. S. Lau, P. L. Huang, J. T. Stull, and R. G. Victor. 1998. Impaired metabolic modulation of alpha-adrenergic vasoconstriction in dystrophin-deficient skeletal muscle. Proc Natl Acad Sci USA. 95:15090-15095.
Thomas, G. D., P. W. Shaul, I. S. Yuhanna, S. C. Froehner, and M. E. Adams. 2003. Vasomodulation by skeletal muscle-derived nitric oxide requires alpha-syntrophin-mediated sarcolemmal localization of neuronal Nitric oxide synthase. Circ Res. 92:554-560.
Tidball, J. G., and M. Wehling-Henricks. 2004. Expression of a NOS transgene in dystrophin-deficient muscle reduces muscle membrane damage without increasing the expression of membrane-associated cytoskeletal proteins. Mol Genet Metab. 82:312-320.
Waite, A., C. L. Tinsley, M. Locke, and D. J. Blake. 2009. The neurobiology of the dystrophin-associated glycoprotein complex. Ann Med. 41:344-359.
Wang, Y., D. C. Newton, and P. A. Marsden. 1999. Neuronal NOS: gene structure, mRNA diversity, and functional relevance. Crit Rev Neurobiol. 13:21-43.
Wang, Z., R. Storb, C. L. Halbert, G. B. Banks, T. M. Butts, E. E. Finn, J. M. Allen, A. D. Miller, J. S. Chamberlain, and S. J. Tapscott. 2012. Successful regional delivery and long-term expression of a dystrophin gene in canine muscular dystrophy: a preclinical model for human therapies. Mol Ther. 20:1501-1507.
Wehling-Henricks, M., M. Oltmann, C. Rinaldi, K. H. Myung, and J. G. Tidball. 2009. Loss of positive allosteric interactions between neuronal nitric oxide synthase and phosphofructokinase contributes to defects in glycolysis and increased fatigability in muscular dystrophy. Hum Mol Genet. 18:3439-3451.
Wehling, M., M. J. Spencer, and J. G. Tidball. 2001. A nitric oxide synthase transgene ameliorates muscular dystrophy in mdx mice. J Cell Biol. 155:123-131.
Whitehead, N. P., C. Pham, O. L. Gervasio, and D. G. Allen. 2008. N-Acetylcysteine ameliorates skeletal muscle pathophysiology in mdx mice. J Physiol. 586:2003-2014.
Wynn, T. A. 2008. Cellular and molecular mechanisms of fibrosis. J Pathol. 214:199-210.
Zhou, L., and D. Y. Zhu. 2009. Neuronal nitric oxide synthase: structure, subcellular localization, regulation, and clinical implications. Nitric Oxide. 20:223-230.
Ziche, M., L. Morbidelli, E. Masini, S. Amerini, H. J. Granger, C. A. Maggi, P. Geppetti, and F. Ledda. 1994. Nitric oxide mediates angiogenesis in vivo and endothelial cell growth and migration in vitro promoted by substance P. J Clin Invest. 94:2036-2044.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed:

1. A fusion polypeptide comprising: an nNOSμ functional fragment and a plasma membrane targeting sequence.

2. The fusion polypeptide of claim 1, wherein the plasma membrane targeting sequence is a k-RAS palmitoylation signal sequence.

3. A peptide comprising: a palmitoylated nNOSμ polypeptide.

4. A vector comprising a nucleotide sequence encoding the fusion polypeptide of claim 1.

5. The vector of claim 4, further comprising a nucleotide sequence encoding a muscle-specific promoter, enhancer, or both.

6. A modified RNA molecule comprising a nucleotide sequence encoding the fusion polypeptide of claim 1, wherein said nucleotide sequence comprises at least two different modified nucleosides.

7. The modified RNA molecule of claim 6, wherein the at least two different modified nucleosides are 5-methylcytidine and pseudouridine.

8. A method of localizing an nNOSμ polypeptide to the sarcolemma comprising introducing the polypeptide of claim 1 to a muscle cell.

9. A method of localizing an nNOSμ polypeptide to the sarcolemma in a subject in need thereof comprising administering the polypeptide of claim 1 to the subject in need thereof.

10. A method of treating a neuromuscular disease in a subject comprising administering the polypeptide of claim 1 to the subject.

11. The method of claim 10 wherein the neuromuscular disease is, one of: Duchenne muscular dystrophy; Becker muscular dystrophy; Limb-girdle muscular dystrophies; Ullrich congenital muscular dystrophy; inflammatory myositis; muscle atrophy; Amyotrophic lateral sclerosis; cachexia; and sarcopenia.

12. A vector comprising a nucleotide sequence encoding the peptide of claim 3.

13. The vector of claim 12, further comprising a nucleotide sequence encoding a muscle-specific promoter, enhancer, or both.

14. A method of localizing an nNOSμ polypeptide to the sarcolemma comprising introducing the vector of claim 12 to a muscle cell.

15. A method of localizing an nNOSμ polypeptide to the sarcolemma comprising introducing the vector of claim 4 to a muscle cell.

16. A method of localizing an nNOSμ polypeptide to the sarcolemma comprising introducing the modified RNA of claim 6 to a muscle cell.

17. A method of localizing an nNOSμ polypeptide to the sarcolemma in a subject in need thereof comprising administering the vector of claim 4 to the subject in need thereof.

18. A method of localizing an nNOSμ polypeptide to the sarcolemma in a subject in need thereof comprising introducing the modified RNA of claim 6 to the subject in need thereof.

19. A method of treating a neuromuscular disease in a subject comprising administering the vector of claim 4 to the subject.

20. A method of treating a neuromuscular disease in a subject comprising administering the modified RNA of claim 6 to the subject.
